SEQUENCE LISTING (210 sequences)

SEQ ID NO: 1 - RNA, 167 nt, Mus musculus
cagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcucc

SEQ ID NO: 2 - RNA, 77 nt, Mus musculus
gaacuggaguuauacgguaaccucguggugguugugaaccaccauguggauggauauugaguuccaaacacuggucc

SEQ ID NO: 3 - RNA, 29 nt, Mus musculus
ccucguggugguugugaaccaccaugugg

SEQ ID NO: 4 - RNA, 61 nt, Mus musculus
guuauacgguaaccucguggugguugugaaccaccauguggauggauauugaguuccaaac

SEQ ID NO: 5 - RNA, 98 nt, Mus musculus
aucccccagaacuggaguuauacgguaaccucguggugguugugaaccaccauguggauggauauugaguuccaaacacugguccugugcaagagcau

SEQ ID NO: 6 - RNA, 129 nt, Mus musculus
gaagagggcauuggaucccccagaacuggaguuauacgguaaccucguggugguugugaaccaccauguggauggauauugaguuccaaacacugguccugugcaagagcauccagugcucuuaagugc

SEQ ID NO: 7 - RNA, 183 nt, Mus musculus
gggcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaagcu

SEQ ID NO: 8 - RNA, 67 nt, Mus musculus
gaacuggaguuauacgguaaccucguggugguugugaaccaccauguggauggauauugaguuccaa

SEQ ID NO: 9 - RNA, 107 nt, Mus musculus
ggaucccccagaacuggaguuauacgguaaccucguggugguugugaaccaccauguggauggauauugaguuccaaacacugguccugugcaagagcauccagugc

SEQ ID NO: 10 - RNA, 127 nt, Mus musculus
agagggcauuggaucccccagaacuggaguuauacgguaaccucguggugguugugaaccaccauguggauggauauugaguuccaaacacugguccugugcaagagcauccagugcucuuaagugc

SEQ ID NO: 11 - RNA, 38 nt, Mus musculus
gguaaccucguggugguugugaaccaccauguggaugg

SEQ ID NO: 12 - RNA, 180 nt, Artificial sequence
  mutated functionally active fragment of inverted SINE B2 transposable element derived from AS Uchl 1
cagugcuagaggaggucagaagagggcauuggaucccccagaacuggaguuauacgauaaccucguggugguugugaaccaccauguggauggauauugaguuccaaacacugguccugugcaagagcauccagugcucuuaagugcugagccaucucuuuagcuccagucucuuaagcu

SEQ ID NO: 13 - RNA, 180 nt, Artificial sequence
  mutated functionally active fragment of inverted SINE B2 transposable element derived from AS Uchl 1
cagugcuagaggaggucagaagagggcauuggaucccccagaacuggaguuauacgcuaaccucguggugguugugaaccaccauguggauggauauugaguuccaaacacugguccugugcaagagcauccagugcucuuaagugcugagccaucucuuuagcuccagucucuuaagcu

SEQ ID NO: 14 - RNA, 180 nt, Artificial sequence
  mutated functionally active fragment of inverted SINE B2 transposable element derived from AS Uchl 1
cagugcuagaggaggucagaagagggcauuggaucccccagaacuggcguuauacgguaaccucguggugguugugaaccaccauguggauggauauugaguuccaaacacugguccugugcaagagcauccagugcucuuaagugcugagccaucucuuuagcuccagucucuuaagcu

SEQ ID NO: 15 - RNA, 180 nt, Artificial sequence
  mutated functionally active fragment of inverted SINE B2 transposable element derived from AS Uchl 1
cagugcuagaggaggucagaagagggcauuggaucccccagaaguggaguuauacgguaaccucguggugguugugaaccaccauguggauggauauugaguuccaaacacugcuccugugcaagagcauccagugcucuuaagugcugagccaucucuuuagcuccagucucuuaagcu

SEQ ID NO: 16 - RNA, 180 nt, Artificial sequence
  mutated functionally active fragment of inverted SINE B2 transposable element derived from AS Uchl 1
cagugcuagaggaggucagaagagggcauuggaucccccagauggugaguuauacgguaaccucguggugguugugaaccaccauguggauggauauugaguuccaaacacgucaccugugcaagagcauccagugcucuuaagugcugagccaucucuuuagcuccagucucuuaagcu

SEQ ID NO: 17 - RNA, 180 nt, Artificial sequence
  Mutated functionally active fragment of inverted SINE B2 transposable element derived from AS Uchl 1
cagugcuagaggaggucagaagagggcauuggaucccccagaacugcacuauacgguaaccucguggugguugugaaccaccauguggauggauauugaguuccaaaugagugguccugugcaagagcauccagugcucuuaagugcugagccaucucuuuagcuccagucucuuaagcu

SEQ ID NO: 18 - RNA, 116 nt, Artificial sequence
  Functionally active fragment of inverted SINE B2 transposable element derived from AS Uchl 1
gggcauuggaucccccagaacuggaguuauacgguaaccucguggugguugugaaccaccauguggauggauauugaguuccaaacacugguccugugcaagagcauccagugcuc

SEQ ID NO: 19 - RNA, 77 nt, Artificial sequence
  Mutated functionally active fragment of inverted SINE B2 transposable element derived from AS Uchl 1
ggacuggaguuauacgguaaccucguggugguugugaaccaccauguggauggauauugaguuccaaacacuggucc

SEQ ID NO: 20 - RNA, 77 nt, Artificial sequence
  Mutated functionally active fragment of inverted SINE B2 transposable element derived from AS Uchl 1
gaacuggcguuauacgguaaccucguggugguugugaaccaccauguggauggauauugaguuccaaacacuggucc

SEQ ID NO: 21 - RNA, 107 nt, Mus musculus
gaugccuuagaaguggaguuaagaguugugagcugccguuuuuugguucugggacucgaacucguuuccucugauacuaucaaccaccaagccaucucuucagcccc

SEQ ID NO: 22 - RNA, 131 nt, Mus musculus
gccagaagaaguugugggauucccuggaacuggagcaaccaacaguuugugugcaccauguggguaaugggaaucgaaccuggguccucuauaagacuggccagugcucuuaacuacugaggugcauuucu

SEQ ID NO: 23 - RNA, 187 nt, Mus musculus
uuauuuuaaauauaugaguauuucaccugcauaggcgcacaguacccacagagacuagaagaggguggcagaucuccugagacuggaguuaaugcuugugagcugccauguggaugcuggaaaucaaacccagguccuuuggaaggcaggcaggugcucuuaaucauggaagcaucucuucagcucc

SEQ ID NO: 24 - RNA, 131 nt, Mus musculus
cagcgacaucagaagaggauauuggaucccauuacagaugguugaaggccaccaugucguugcugggaaugaacucaagaccucuggaagagcagucagugcucuuaaccucugagccaucucuccagccc

SEQ ID NO: 25 - RNA, 114 nt, Mus musculus
auccccuccaaagcucaagaugguuguaagccacccugugauugcugggauuugaacucaagaccuccggaagagcaauuagugcucuuaaccgcugagcaaucucuccagccc

SEQ ID NO: 26 - RNA, 357 nt, Mus musculus
gugcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccuuauuuuaaauauaugaguauuucaccugcauaggcgcacaguacccacagagacuagaagaggguggcagaucuccugagacuggaguuaaugcuugugagcugccauguggaugcuggaaaucaaacccagguccuuuggaaggcaggcaggugcucuuaaucauggaagcaucucuucagcucc

SEQ ID NO: 27 - RNA, 532 nt, Mus musculus
gugcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccgugcgaauucggugcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccgugcgaauucggugcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcucc

SEQ ID NO: 28 - RNA, 228 nt, Mus musculus
uuuuuuuaaaaauuuauuuuuauuuuauguguaugaguguuuugccugcauguaugucuguguaccacgugcgugccuggugcccgcggaggccagaagagggcgucggauccccuggaacuggaguuacagaugguugugagccgccaugugggugcugggaaucgaacccggguccucuggaagagcagccagugcucuuaaccgcugagccaucucuccagcccc

SEQ ID NO: 29 - RNA, 214 nt, Mus musculus
uuuuuuuuacuuguauagguguuuugccugcauguguaucuaucuauguaccgaauauguuccugguauccacagagaccaaaaguggauguuguaucuccugaaauuggagucauagacaguuaugagcugccauuugagugcuuggaauagaacccagguccucuuaaagagcauccagugcucuuaaaaacugagacaucucuguagccuc

SEQ ID NO: 30 - RNA, 200 nt, Mus musculus
uuuauuuugcuuuaugugucugaguguuugcuugaauguaugucuguguaccacgccuguaccuugugccuucagaguugagaggagggcauaggaucuccuggaacuggaauugcaggugguugugagccacccuguggguccuggggaccauacuccagcaagaacaucaugugcucuuaauuccugagucuccaacc

SEQ ID NO: 31 - RNA, 214 nt, Mus musculus
uuuauuuacuuaucuuuauguguaugaguguguugucagacuguuaugucugugugucacaugcaugccugcuguucauggaguccagaagagggcaucggauccccuggaacuggaguuacagaugaguggccaugugaauguuaagaaccaaaccuggguccucugaaagagcagacaaugcucuuaacuacugagcugucucuccagcccc

SEQ ID NO: 32 - RNA, 205 nt, Mus musculus
uuauuuuauucguguaaguguuuugccagcaucuaugucuucgcacuaugugcaggucuggugccugagggguccagacgagagcacugggucuccgggaacuggaguuacagaucauugugagccaccaugugggugcagggaaucgaaccugggaccucuggaggagcagccacugcucuuaaccacuacacuauuucuccag

SEQ ID NO: 33 - RNA, 121 nt, Mus musculus
ucuguggaccacuguguacagaagccugagaaggcuagcagauccccagaacuggaacugugagacgcugugcuauggaggugcuaggaacugaaaauggauggguccucugcaagagcag

SEQ ID NO: 34 - RNA, 191 nt, Mus musculus
uuguuuuaauugaauggcuauaggguguuucuucuguauguauaucuauguuugguaccuacagaggcaucagauccucuggaacuguaguugcugacaguugugagcugucauggggaugcuggaauugaaccuggauccuaugaaagaacagccaguguucuuaaccgcugagcuaucucuccaggccc

SEQ ID NO: 35 - RNA, 205 nt, Mus musculus
uuuuuuuuuuaauuuuaaaaaaaaagauuuuauuuauuuauuuuauauaugaugaguacacugucacucuuuucagacacccuagaaaaggggggcaucagaucccauuacagaugguugugagccacaugguugcugggaauugaccucaggaccucugaaagagcagucagugcucucaaccuuugagucaucucuccagccc

SEQ ID NO: 36 - RNA, 190 nt, Mus musculus
auguauaucuguaaugggacauacucacauacaugggcacgugaguauaaaaggccagaagagagcacuggacccucuggaguugagauucuaagcaguugugaaccaucugauguaggugcugggaacugaacuuggguccuuugcuagagaaguaugucucuuaaccacugagccguaucuccauccc

SEQ ID NO: 37 - RNA, 169 nt, Mus musculus
uaaagauuuauucauuaaguacacuguagcuaucuucagacgcaucagaagagggcgucagaucucuuuacaggugguugugagccaccaugugguugcuggaauuugaacucaggaccuucaaaagagcagucaguguucuuaaccgcugagccaucucuccaacccc

SEQ ID NO: 38 - RNA, 159 nt, Mus musculus
uuauuuauuauaaguacacuguagcugucuucagacacaacaaaagagggcgucagaucucauuacaggugguugagccaccaugugguugcugggauuugaacucaggaccuucagaacagucagugcucuuacccacugagccagcgagccagcccc

SEQ ID NO: 39 - RNA, 77 nt, Artificial sequence
  Mutated functionally active fragment of inverted SINE B2 transposable element derived from AS Uchl 1
gaacuggaguuauacgguaaccucguggugguugggaaccaccauguggauggauauugaguuccaaacacuggucc

SEQ ID NO: 40 - RNA, 77 nt, Artificial sequence
  Mutated functionally active fragment of inverted SINE B2 transposable element derived from AS Uchl 1
gaacuggaguuauacgguaaccucguggugguucccaaccaccauguggauggauauugaguuccaaacacuggucc

SEQ ID NO: 41 - RNA, 77 nt, Artificial sequence
  Mutated functionally active fragment of inverted SINE B2 transposable element derived from AS Uchl 1
ggaccggaguuauacgguaaccgcguggugguugugaaccaccacgcggauggauauugaguuccaaacaccggucc

SEQ ID NO: 42 - RNA, 77 nt, Artificial sequence
  Mutated functionally active fragment of inverted SINE B2 transposable element derived from AS Uchl 1
gaacuagaguuauacgguaaccacauggugguugugaaccaccauguggauggauauugaguuccaaacacuaguuc

SEQ ID NO: 43 - RNA, 206 nt, Artificial sequence
  Functionally active fragment of inverted SINE B2 transposable element derived from AS Uchl 1
uuauuuuaaauauaugaguauuucaccugcauaggcgcacaguacccacagagacuagaagaggguaguagauccccuagaacuggaguuauacgguaaccucguggugguugugagcuaccauguggauggauacugggaaucaaacccagguccuguggaaggcaggcaggugcucucaagcacugagccaucucuucagcucc

SEQ ID NO: 44 - RNA, 206 nt, Artificial sequence
  Functionally active fragment of inverted SINE B2 transposable element derived from AS Uchl 1
uuauuuuaaauauaugaguauuucaccugcauaggcgcacagugcucaaggagaucagaagagggcaucagaucuccugagacuggaguuauacgguaaccucgugaugguugugaacuaccauguggauggauauugaguuccaaacacagguccugugcaagagcagcaggugcucuuaagcacggaaccaucucuuuagcucc

SEQ ID NO: 45 - RNA, 156 nt, Artificial sequence
  Functionally active fragment of inverted SINE B2 transposable element derived from AS Uchl 1
gaggcuagaagaggguaucagauccccugagacuggaguuauacgguaaccucguggugguugugagccaccauguggauggauacugagaaccaaacccugguccugugcaagagcaucaggugcucuuaagcacggaaccaucucuucagcucc

SEQ ID NO: 46 - RNA, 68 nt, Artificial sequence
  Functionally active fragment of inverted SINE B2 transposable element derived from AS Uchl 1
guccugugcaagagcaucgaacucggugcucuuaagcacagaagccaccaagccaucucuucagcccc

SEQ ID NO: 47 - RNA, 110 nt, Artificial sequence
  Functionally active fragment of inverted SINE B2 transposable element derived from AS Uchl 1
cagugcuagaggaggucagaagagggcaucccccagccucguggugguugugaaccaccauguggcugugcaagagcaugcucuuaagugcugagccaucucuuuagcuc

SEQ ID NO: 48 - RNA, 126 nt, Artificial sequence
  Functionally active fragment of inverted SINE B2 transposable element derived from AS Uchl 1
gagggcauuggaucccccagaacuggaguuauacgguaaccucguggugguugugaaccaccauguggauggauauugaguuccaaacacugguccugugcaagagcauccagugcucuuaagugc

SEQ ID NO: 49 - RNA, 89 nt, Artificial sequence
  Functionally active fragment of inverted SINE B2 transposable element derived from AS Uchl 1
ggaucccccagaacuggaguuauacgguaaccucguggugguugugaaccaccauguggauggauauugaguuccaaacacugguccug

SEQ ID NO: 50 - RNA, 72 nt, Artificial sequence
  Functionally active fragment of inverted SINE B2 transposable element derived from AS Uchl 1
ugcuagaggaggucagaagagggcauuggaugcaaauccagugcucuuaagugcugagccaucucuuuagcu

SEQ ID NO: 51 - RNA, 94 nt, Artificial sequence
  Functionally active fragment of inverted SINE B2 transposable element derived from AS Uchl 1
gagggcauuggaucccccagaacuggaguuauacgguaacgauggauauugaguuccaaacacugguccugugcaagagcauccagugcucuua

SEQ ID NO: 52 - RNA, 383 nt, Hepatitis C virus
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguugggucgcgaaaggccuugugguacugccugauagggugcuugcgagugccccgggaggucucguagaccgugcaccaugagcacgaauccuaaaccucaaagaaaaaccaaacguaac

SEQ ID NO: 53 - RNA, 383 nt, Hepatitis C virus
guuacguuugguuuuucuuugagguuuaggauucgugcucauggugcacggucuacgagaccucccggggcacucgcaagcacccuaucaggcaguaccacaaggccuuucgcgacccaacacuacucggcuagcagucuugcgggggcacgcccaaaucuccaggcauugagcggguuuauccaagaaaggacccggucguccuggcaauuccgguguacucaccgguuccgcagaccacuauggcucucccgggaggggggguccuggaggcugcacgacacucauacuaacgccauggcuagacgcuuucugcgugaagacaguaguuccucacaggggagugauucaugguggagugucgcccccaucagggggcuggc

SEQ ID NO: 54 - RNA, 312 nt, Human poliovirus
augagucuggacaucccucaccggugacggugguccaggcugcguuggcggccuaccuauggcuaacgccaugggacgcuaguugugaacaaggugugaagagccuauugagcuacauaagaauccuccggccccugaaugcggcuaaucccaaccucggagcagguggucacaaaccagugauuggccugucguaacgcgcaaguccguggcggaaccgacuacuuuggguguccguguuuccuuuuauuuuauuguggcugcuuauggugacaaucacagauuguuaucauaaagcgaauuggauuggcc

SEQ ID NO: 55 - RNA, 312 nt, Human poliovirus
ggccaauccaauucgcuuuaugauaacaaucugugauugucaccauaagcagccacaauaaaauaaaaggaaacacggacacccaaaguagucgguuccgccacggacuugcgcguuacgacaggccaaucacugguuugugaccaccugcuccgagguugggauuagccgcauucaggggccggaggauucuuauguagcucaauaggcucuucacaccuuguucacaacuagcgucccauggcguuagccauagguaggccgccaacgcagccuggaccaccgucaccggugagggauguccagacucau

SEQ ID NO: 56 - RNA, 576 nt, Encephalomyocarditis virus
cccccccucucccuccccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaa

SEQ ID NO: 57 - RNA, 576 nt, Encephalomyocarditis virus
uuaucaucguguuuuucaaaggaaaaccacguccccgugguucggggggccuagacguuuuuuaaccucgacuaaacacauguaaagcaugugcaccgaggccccagaucagaucccauacaaugggguaccuucugggcauccuucagccccuuguugaauacgcuugaggagagccauuugacucuuuccacaacuauccaacucacaacguggcacugggguugugccgccuuugcagguguaucuuauacacguggcuuuuggccgcagaggcaccugucgccaggugggggguuccgcugccugcaaagggucgcuacagacguuguuugucuucaagaagcuuccagaggaacugcuuccuucacgacauucaacagaccuugcauuccuuuggcgagaggggaaagaccccuaggaaugcucgucaagaagacagggccagguuuccgggcccucacauugccaaaagacggcaauaugguggaaaauaacauauagacaaacgcacaccggccuuauuccaagcggcuucggccaguaacguuaggggggggggagggagaggggggg

SEQ ID NO: 58 - RNA, 192 nt, Cricket paralysis virus
aaagcaaaaaugugaucuugcuuguaaauacaauuuugagagguuaauaaauuacaaguagugcuauuuuuguauuuagguuagcuauuuagcuuuacguuccaggaugccuaguggcagccccacaauauccaggaagcccucucugcgguuuuucagauuagguagucgaaaaaccuaagaaauuuaccu

SEQ ID NO: 59 - RNA, 192 nt, Cricket paralysis virus
agguaaauuucuuagguuuuucgacuaccuaaucugaaaaaccgcagagagggcuuccuggauauuguggggcugccacuaggcauccuggaacguaaagcuaaauagcuaaccuaaauacaaaaauagcacuacuuguaauuuauuaaccucucaaaauuguauuuacaagcaagaucacauuuuugcuuu

SEQ ID NO: 60 - RNA, 231 nt, Homo sapiens
cagagauccaggggaggcgccugugaggcccggaccugccccggggcgaaggguauguggcgagacagagcccugcaccccuaauucccgguggaaaacuccuguugccguuucccuccaccggccuggagucucccagucuugucccggcagugccgcccuccccacuaagaccuaggcgcaaaggcuuggcucaugguugacagcucagagagagaaagaucugaggga

SEQ ID NO: 61 - RNA, 231 nt, Homo sapiens
ucccucagaucuuucucucucugagcugucaaccaugagccaagccuuugcgccuaggucuuaguggggagggcggcacugccgggacaagacugggagacuccaggccgguggagggaaacggcaacaggaguuuuccaccgggaauuaggggugcagggcucugucucgccacauacccuucgccccggggcagguccgggccucacaggcgccuccccuggaucucug

SEQ ID NO: 62 - RNA, 460 nt, Homo sapiens
acuuuuggugggcauuuaaaaauguguguguauguguauauauguauguguauguauguguauauauguauauguauguauguaucgcguguauguguguauguaugcauguguauguauguauaugcauguauguguauguguauauauguauguguguguauguauauguguguguauguguauguguguguguauguguguguguauguauguauguauguauauguauuauacacauauacacauauugguuuuuuuaaucauuugagaguuaguugaagauaaaaacccaucaccccuaaauguauuccaaagaauaagaacauuguuuuauacauagcacacuuaacaaaaucaagaaauuuaacauuaauacaguacuguuaccuaauccguagucgauuuucaaauuuugucaguuguuccaauaauguccuuuauauauuccccgcccagc

SEQ ID NO: 63 - RNA, 460 nt, Homo sapiens
gcugggcggggaauauauaaaggacauuauuggaacaacugacaaaauuugaaaaucgacuacggauuagguaacaguacuguauuaauguuaaauuucuugauuuuguuaagugugcuauguauaaaacaauguucuuauucuuuggaauacauuuaggggugauggguuuuuaucuucaacuaacucucaaaugauuaaaaaaaccaauauguguauauguguauaauacauauacauacauacauacauacacacacacauacacacacacauacacauacacacacauauacauacacacacauacauauauacacauacacauacaugcauauacauacauacacaugcauacauacacacauacacgcgauacauacauacauauacauauauacacauacauacacauacauauauacacauacacacacauuuuuaaaugcccaccaaaagu

SEQ ID NO: 64 - RNA, 395 nt, Homo sapiens
aauuccagcgagaggcagagggagcgagcgggcggccggcuaggguggaagagccgggcgagcagagcugcgcugcgggcguccugggaagggagauccggagcgaauagggggcuucgccucuggcccagcccucccgcuugaucccccaggccagcgguccgcaacccuugccgcauccacgaaacuuugcccauagcagcgggcgggcacuuugcacuggaacuuacaacacccgagcaaggacgcgacucucccgacgcggggaggcuauucugcccauuuggggacacuuccccgccgcugccaggacccgcuucucugaaaggcucuccuugcagcugcuuagacgcuggauuuuuuucggguaguggaaaaccagcagccucccgcga

SEQ ID NO: 65 - RNA, 395 nt, Homo sapiens
ucgcgggaggcugcugguuuuccacuacccgaaaaaaauccagcgucuaagcagcugcaaggagagccuuucagagaagcggguccuggcagcggcggggaaguguccccaaaugggcagaauagccuccccgcgucgggagagucgcguccuugcucggguguuguaaguuccagugcaaagugcccgcccgcugcuaugggcaaaguuucguggaugcggcaaggguugcggaccgcuggccugggggaucaagcgggagggcugggccagaggcgaagcccccuauucgcuccggaucucccuucccaggacgcccgcagcgcagcucugcucgcccggcucuuccacccuagccggccgcccgcucgcucccucugccucucgcuggaauu

SEQ ID NO: 66 - RNA, 48 nt, Homo sapiens
gggcacuuugcacuggaacuuacaacacccgagcaaggacgcgacucu

SEQ ID NO: 67 - RNA, 48 nt, Homo sapiens
agagucgcguccuugcucggguguuguaaguuccagugcaaagugccc

SEQ ID NO: 68 - RNA, 71 nt, Homo sapiens
guacugacaucguagauggaaaucauaaacugacucuugguuugauuuggaauauaauccuccacuggcag

SEQ ID NO: 69 - RNA, 71 nt, Homo sapiens
cugccaguggaggauuauauuccaaaucaaaccaagagucaguuuaugauuuccaucuacgaugucaguac

SEQ ID NO: 70 - DNA, 18 bp, Homo sapiens
gcatggcttcagccgagt

SEQ ID NO: 71 - DNA, 22 bp, Homo sapiens
gcatggcttcagccgagttaca

SEQ ID NO: 72 - DNA, 18 bp, Homo sapiens
ggcttcagccgagttaca

SEQ ID NO: 73 - DNA, 20 bp, Homo sapiens
ggcttcagccgagttacagg

SEQ ID NO: 74 - DNA, 24 bp, Homo sapiens
gcatggcttcagccgagttacagg

SEQ ID NO: 75 - DNA, 18 bp, Homo sapiens
ccatgctaaacttgacaa

SEQ ID NO: 76 - DNA, 18 bp, Homo sapiens
gctaaacttgacaaactc

SEQ ID NO: 77 - DNA, 22 bp, Homo sapiens
ccatgctaaacttgacaaactc

SEQ ID NO: 78 - DNA, 18 bp, Homo sapiens
ccataaaggtactgaagc

SEQ ID NO: 79 - DNA, 18 bp, Homo sapiens
aaaggtactgaagctggg

SEQ ID NO: 80 - DNA, 18 bp, Homo sapiens
cggcggcacctcggatcg

SEQ ID NO: 81 - DNA, 38 bp, Homo sapiens
taaagtcccgtccggcggcggcacctcggatcgggtct

SEQ ID NO: 82 - DNA, 40 bp, Homo sapiens
cttaaagtcccgtccggcggcggcacctcggatcgggtct

SEQ ID NO: 83 - DNA, 26 bp, Homo sapiens
cttaaagtcccgtccggcggcggcac

SEQ ID NO: 84 - DNA, 14 bp, Homo sapiens
cttaaagtcccgtc

SEQ ID NO: 85 - DNA, 18 bp, Homo sapiens
tcatcttaaagtcccgtc

SEQ ID NO: 86 - DNA, 19 bp, Homo sapiens
tcatcttaaagtcccgtcc

SEQ ID NO: 87 - DNA, 20 bp, Homo sapiens
tcatcttaaagtcccgtccg

SEQ ID NO: 88 - DNA, 31 bp, Homo sapiens
tcatcttaaagtcccgtccggcggcggcacc

SEQ ID NO: 89 - DNA, 44 bp, Homo sapiens
tcatcttaaagtcccgtccggcggcggcacctcggatcgggtct

SEQ ID NO: 90 - DNA, 40 bp, Homo sapiens
aacttcatcttaaagtcccgtccggcggcggcacctcgga

SEQ ID NO: 91 - DNA, 70 bp, Homo sapiens
tcccataacttcatcttaaagtcccgtccggcggcggcacctcggatcgggtctccgcagaccctaggtt

SEQ ID NO: 92 - DNA, 31 bp, Homo sapiens
catcccataacttcatcttaaagtcccgtcc

SEQ ID NO: 93 - DNA, 21 bp, Homo sapiens
tcttaaagtcccgtccggcgg

SEQ ID NO: 94 - DNA, 18 bp, Homo sapiens
gcatatttgagtcactgc

SEQ ID NO: 95 - DNA, 25 bp, Homo sapiens
atttgagtcactgctcagcgcgaag

SEQ ID NO: 96 - DNA, 46 bp, Homo sapiens
ccatgacatcatcgaactgatcaggataatcctctggcatatttga

SEQ ID NO: 97 - DNA, 18 bp, Homo sapiens
ccatgacatcatcgaact

SEQ ID NO: 98 - DNA, 18 bp, Homo sapiens
acatgcctgccctacttt

SEQ ID NO: 99 - DNA, 25 bp, Homo sapiens
tcatcttaaagtcccgtccggcggc

SEQ ID NO: 100 - DNA, 21 bp, Homo sapiens
cttaaagtcccgtccggcggc

SEQ ID NO: 101 - DNA, 21 bp, Homo sapiens
acgacatcccataacttcatc

SEQ ID NO: 102 - DNA, 18 bp, Homo sapiens
acatcccataacttcatc

SEQ ID NO: 103 - DNA, 14 bp, Homo sapiens
atttgagtcactgc

SEQ ID NO: 104 - DNA, 18 bp, Homo sapiens
atttgagtcactgctcag

SEQ ID NO: 105 - DNA, 24 bp, Homo sapiens
gcatatttgagtcactgctcagcg

SEQ ID NO: 106 - DNA, 40 bp, Homo sapiens
gggactgcggctagggccggcgggtctggatggcgggtgc

SEQ ID NO: 107 - DNA, 18 bp, Homo sapiens
cccgtgcgcgctggggcc

SEQ ID NO: 108 - DNA, 29 bp, Homo sapiens
ccgtgcgcgctggggccacggctggaggg

SEQ ID NO: 109 - DNA, 38 bp, Homo sapiens
cccgtgcgcgctggggccacggctggagggactgcggc

SEQ ID NO: 110 - DNA, 40 bp, Homo sapiens
cgcccgtgcgcgctggggccacggctggagggactgcggc

SEQ ID NO: 111 - DNA, 14 bp, Homo sapiens
cgcccgtgcgcgct

SEQ ID NO: 112 - DNA, 18 bp, Homo sapiens
ccatcgcccgtgcgcgct

SEQ ID NO: 113 - DNA, 19 bp, Homo sapiens
ccatcgcccgtgcgcgctg

SEQ ID NO: 114 - DNA, 20 bp, Homo sapiens
ccatcgcccgtgcgcgctgg

SEQ ID NO: 115 - DNA, 44 bp, Homo sapiens
ccatcgcccgtgcgcgctggggccacggctggagggactgcggc

SEQ ID NO: 116 - DNA, 40 bp, Homo sapiens
ttcgccatcgcccgtgcgcgctggggccacggctggaggg

SEQ ID NO: 117 - DNA, 70 bp, Homo sapiens
gtcgccttcgccatcgcccgtgcgcgctggggccacggctggagggactgcggctagggccggcgggtct

SEQ ID NO: 118 - DNA, 18 bp, Homo sapiens
ccatcaccacctcctcgc

SEQ ID NO: 119 - DNA, 40 bp, Homo sapiens
caccacctcctcgcgcgcgccggcgtgcacggtgcacacg

SEQ ID NO: 120 - DNA, 18 bp, Homo sapiens
gcatggtgcggttctccg

SEQ ID NO: 121 - DNA, 50 bp, Homo sapiens
ggtgcggttctccgagatggagaggttccggttgagaaccagcctatagt

SEQ ID NO: 122 - DNA, 18 bp, Homo sapiens
ccaatgccactttgccta

SEQ ID NO: 123 - DNA, 18 bp, Homo sapiens
cggttgcggacactgagc

SEQ ID NO: 124 - DNA, 18 bp, Homo sapiens
cacggtgtcctccttccg

SEQ ID NO: 125 - DNA, 44 bp, Homo sapiens
tgtcctccttccgcttgaactccaccacggcgctggcggtgtcg

SEQ ID NO: 126 - DNA, 18 bp, Homo sapiens
gccatcgcccgtgcgcgc

SEQ ID NO: 127 - DNA, 24 bp, Homo sapiens
cctccttccgcttgaactccacca

SEQ ID NO: 128 - DNA, 17 bp, Homo sapiens
catcgcccgtgcgcgct

SEQ ID NO: 129 - DNA, 15 bp, Homo sapiens
ccatcgcccgtgcgc

SEQ ID NO: 130 - DNA, 18 bp, Homo sapiens
tcgccatcgcccgtgcgc

SEQ ID NO: 131 - DNA, 20 bp, Homo sapiens
cttcgccatcgcccgtgcgc

SEQ ID NO: 132 - DNA, 18 bp, Homo sapiens
gacgtcgccttcgccatc

SEQ ID NO: 133 - DNA, 20 bp, Homo sapiens
cggacgtcgccttcgccatc

SEQ ID NO: 134 - DNA, 24 bp, Homo sapiens
gcaccggacgtcgccttcgccatc

SEQ ID NO: 135 - DNA, 20 bp, Homo sapiens
caccatcaccacctcctcgc

SEQ ID NO: 136 - DNA, 18 bp, Homo sapiens
ggaagggcaccatcacca

SEQ ID NO: 137 - DNA, 15 bp, Homo sapiens
aagggcaccatcacc

SEQ ID NO: 138 - DNA, 18 bp, Homo sapiens
gaggatccacgtcggcga

SEQ ID NO: 139 - DNA, 18 bp, Homo sapiens
cctcagggtcattagatg

SEQ ID NO: 140 - DNA, 38 bp, Homo sapiens
cctcagggtcattagatgaagagaagaccacaggggtt

SEQ ID NO: 141 - DNA, 40 bp, Homo sapiens
cccctcagggtcattagatgaagagaagaccacaggggtt

SEQ ID NO: 142 - DNA, 44 bp, Homo sapiens
ccatcccctcagggtcattagatgaagagaagaccacaggggtt

SEQ ID NO: 143 - DNA, 14 bp, Homo sapiens
cccctcagggtcat

SEQ ID NO: 144 - DNA, 18 bp, Homo sapiens
ccatcccctcagggtcat

SEQ ID NO: 145 - DNA, 19 bp, Homo sapiens
ccatcccctcagggtcatt

SEQ ID NO: 146 - DNA, 20 bp, Homo sapiens
ccatcccctcagggtcatta

SEQ ID NO: 147 - DNA, 40 bp, Homo sapiens
aactccatcccctcagggtcattagatgaagagaagacca

SEQ ID NO: 148 - DNA, 70 bp, Homo sapiens
cttgaaaactccatcccctcagggtcattagatgaagagaagaccacaggggttccagagtctctgaagg

SEQ ID NO: 149 - DNA, 40 bp, Homo sapiens
gatgcttaccctactcaaaggcttgggacattcctctctg

SEQ ID NO: 150 - DNA, 18 bp, Homo sapiens
ccatccgtcgcccactgc

SEQ ID NO: 151 - DNA, 40 bp, Homo sapiens
ccgtcgcccactgcgtgtactctcatagcggctgaaggta

SEQ ID NO: 152 - DNA, 54 bp, Homo sapiens
atactcagctccatccgtcgcccactgcgtgtactctcatagcggctgaaggta

SEQ ID NO: 153 - DNA, 18 bp, Homo sapiens
gcatccctcgatcccagg

SEQ ID NO: 154 - DNA, 18 bp, Homo sapiens
caggggtatcttgagctt

SEQ ID NO: 155 - DNA, 18 bp, Homo sapiens
gtatcttgagcttggtat

SEQ ID NO: 156 - DNA, 18 bp, Homo sapiens
actcagctccatccgtcg

SEQ ID NO: 157 - DNA, 30 bp, Homo sapiens
ctggccatgggtacccggatgatgttatat

SEQ ID NO: 158 - DNA, 18 bp, Mus musculus
tgctccacacggctccct

SEQ ID NO: 159 - DNA, 38 bp, Mus musculus
ctaaagatgtatttttgctccacacggctccctaggct

SEQ ID NO: 160 - DNA, 30 bp, Mus musculus
tatttttgctccacacggctccctaggctt

SEQ ID NO: 161 - DNA, 18 bp, Mus musculus
ctaaagatgtatttttgc

SEQ ID NO: 162 - DNA, 40 bp, Mus musculus
ggctaaagatgtatttttgctccacacggctccctaggct

SEQ ID NO: 163 - DNA, 27 bp, Mus musculus
ggctaaagatgtatttttgctccacac

SEQ ID NO: 164 - DNA, 14 bp, Mus musculus
ggctaaagatgtat

SEQ ID NO: 165 - DNA, 18 bp, Mus musculus
ccatggctaaagatgtat

SEQ ID NO: 166 - DNA, 19 bp, Mus musculus
ccatggctaaagatgtatt

SEQ ID NO: 167 - DNA, 20 bp, Mus musculus
ccatggctaaagatgtattt

SEQ ID NO: 168 - DNA, 44 bp, Mus musculus
ccatggctaaagatgtatttttgctccacacggctccctaggct

SEQ ID NO: 169 - DNA, 40 bp, Mus musculus
acatccatggctaaagatgtatttttgctccacacggctc

SEQ ID NO: 170 - DNA, 70 bp, Mus musculus
tcatgaacacatccatggctaaagatgtatttttgctccacacggctccctaggcttctgaagaactccg

SEQ ID NO: 171 - DNA, 42 bp, Mus musculus
tcatgaacacatccatggctaaagatgtatttttgctccaca

SEQ ID NO: 172 - DNA, 21 bp, Mus musculus
tggctaaagatgtatttttgc

SEQ ID NO: 173 - DNA, 18 bp, Mus musculus
tcatgaacacatccatgg

SEQ ID NO: 174 - DNA, 40 bp, Mus musculus
gaacacatccatggctaaagatgtatttttgctccacacg

SEQ ID NO: 175 - DNA, 18 bp, Mus musculus
ctcctcacccttgcccat

SEQ ID NO: 176 - DNA, 37 bp, Mus musculus
tacccctcctcacccttgcccatctggtccttcttga

SEQ ID NO: 177 - DNA, 46 bp, Mus musculus
gcatttcataagcctcactgccaggatccacaggcatgtcttccag

SEQ ID NO: 178 - DNA, 18 bp, Mus musculus
ttttggaacctacataga

SEQ ID NO: 179 - DNA, 44 bp, Mus musculus
cactgttgtcactccatgaaccactccttccttagttttggaac

SEQ ID NO: 180 - DNA, 18 bp, Homo sapiens
tacaccacactgtcgtcg

SEQ ID NO: 181 - DNA, 38 bp, Homo sapiens
ctaatgaattcctttacaccacactgtcgtcgaatggc

SEQ ID NO: 182 - DNA, 30 bp, Homo sapiens
ttcctttacaccacactgtcgtcgaatggc

SEQ ID NO: 183 - DNA, 18 bp, Homo sapiens
ctaatgaattcctttaca

SEQ ID NO: 184 - DNA, 40 bp, Homo sapiens
ggctaatgaattcctttacaccacactgtcgtcgaatggc

SEQ ID NO: 185 - DNA, 26 bp, Homo sapiens
ggctaatgaattcctttacaccacac

SEQ ID NO: 186 - DNA, 14 bp, Homo sapiens
ggctaatgaattcc

SEQ ID NO: 187 - DNA, 18 bp, Homo sapiens
ccatggctaatgaattcc

SEQ ID NO: 188 - DNA, 19 bp, Homo sapiens
ccatggctaatgaattcct

SEQ ID NO: 189 - DNA, 20 bp, Homo sapiens
ccatggctaatgaattcctt

SEQ ID NO: 190 - DNA, 44 bp, Homo sapiens
ccatggctaatgaattcctttacaccacactgtcgtcgaatggc

SEQ ID NO: 191 - DNA, 40 bp, Homo sapiens
acatccatggctaatgaattcctttacaccacactgtcgt

SEQ ID NO: 192 - DNA, 70 bp, Homo sapiens
tcatgaatacatccatggctaatgaattcctttacaccacactgtcgtcgaatggccactcccagttctc

SEQ ID NO: 193 - DNA, 41 bp, Homo sapiens
tcatgaatacatccatggctaatgaattcctttacaccaca

SEQ ID NO: 194 - DNA, 21 bp, Homo sapiens
tggctaatgaattcctttaca

SEQ ID NO: 195 - DNA, 18 bp, Homo sapiens
tcatgaatacatccatgg

SEQ ID NO: 196 - DNA, 40 bp, Homo sapiens
gaatacatccatggctaatgaattcctttacaccacactg

SEQ ID NO: 197 - DNA, 46 bp, Homo sapiens
gcatttcataagcctcattgtcaggatccacaggcatatcttccag

SEQ ID NO: 198 - DNA, 18 bp, Homo sapiens
gcatatcttccagaattc

SEQ ID NO: 199 - DNA, 18 bp, Homo sapiens
ttttggagcctacataga

SEQ ID NO: 200 - DNA, 44 bp, Homo sapiens
gagcctacatagagaacaccctcttttgtctttcctgctgcttc

SEQ ID NO: 201 - DNA, 18 bp, Homo sapiens
ttcttcattcttgcccaa

SEQ ID NO: 202 - DNA, 18 bp, Homo sapiens
gaatacatccatggctaa

SEQ ID NO: 203 - RNA, 13 nt, Artificial Sequence
  synthetic example of a spacer/linker sequence
aucugcagaauuc

SEQ ID NO: 204 - RNA, 6 nt, Artificial Sequence
  synthetic example of a spacer/linker sequence
gaauuc

SEQ ID NO: 205 - DNA, 14 bp, Artificial Sequence
  GDNF BDI (-14/-1) M1
cttaaagtcccgtc

SEQ ID NO: 206 - DNA, 18 bp, Artificial Sequence
  GDNF BDII (-14/+4) M1
tcatcttaaagtcccgtc

SEQ ID NO: 207 - DNA, 19 bp, Artificial Sequence
  GDNF BDIII (-15/+4 M1)
tcatcttaaagtcccgtcc

SEQ ID NO: 208 - DNA, 18 bp, Artificial Sequence
  GDNF BDIV (-14/+4 M2)
gcatatttgagtcactgc

SEQ ID NO: 209 - DNA, 25 bp, Artificial Sequence
  GDNF BDV (-25/-1 M2)
atttgagtcactgctcagcgcgaag

SEQ ID NO: 210 - DNA, 46 bp, Artificial Sequence; identical to SEQ ID NO: 96
  GDNF BDVI (-42/+4 M3)

(19) United States
(12) Patent Application Publication
(10) Pub. No.: US 2024/0200073 A1
(43) Pub. Date: Jun. 20, 2024

(54) FUNCTIONAL NUCLEIC ACID MOLECULES DIRECTED TO TARGETS FOR NERVOUS SYSTEM DISORDERS

(71) Applicants: TRANSINE THERAPEUTICS LIMITED, Cambridge (GB); FONDAZIONE ISTITUTO ITALIANO DI TECNOLOGIA, Genova (IT)

(72) Inventors: Laura GRASSO, Royston (GB); Carlotta BON, Genova (IT); Stefano GUSTINCICH, Genova (IT); Stefano ESPINOZA, Genova (IT); Salvatore PULCRANO, Genova (IT)

(21) Appl. No.: 18/551,618

(22) PCT Filed: Mar. 21, 2022
(86) PCT No.: PCT/GB2022/050708

§ 371 Date: Sep. 21, 2023

(30) Foreign Application Priority Data
Mar. 22, 2021 (EP) 21425011.0

(51) Int. Cl. C12N 15/113; C12N 15/63

(52) U.S. Cl. CPC C12N 15/113; C12N 15/63; C12N 2310/11

(57) ABSTRACT

The invention relates to therapeutic agents which enhance protein translation of an endogenous mRNA sequence, particularly for use in a method of treating a disease or disorder of the nervous system. In particular, the therapeutic agent is a functional nucleic acid molecule comprising at least one target determinant sequence and at least one regulatory sequence.

### FIELD OF THE INVENTION

The present invention relates to therapeutic agents for use in a method of treating a disease or disorder of the nervous system, wherein the therapeutic agent increases translation of an endogenous mRNA sequence selected from the group consisting of: a Nurr1, GDNF, cRET or GBA mRNA sequence. Also provided are functional nucleic acid molecules comprising: at least one target binding sequence comprising a sequence reverse complementary to a target mRNA sequence selected from the group consisting of: a Nurr1, GDNF, cRET, or GBA mRNA sequence; and at least one regulatory sequence, as well as methods for use of said functional nucleic acid molecule, particularly in the treatment of diseases or disorders of the nervous system. Also provided are functional nucleic acid molecules comprising: at least one target binding sequence comprising a sequence reverse complementary to a SNCA mRNA sequence; and at least one regulatory sequence, as well as methods for use of said functional nucleic acid molecule, particularly in the generation of animal models.

### BACKGROUND OF THE INVENTION

Parkinson's Disease (PD) is one of the most common neurodegenerative disorders, and it is caused by loss of the dopamine (DA) neurons of the substantia nigra pars compacta (SNpc). It manifests its symptomatology mainly as motor-related deficits, such as rigidity, tremor, and bradykinesia (Meissner et al. (2011) Nat. Reviews Drug Discovery 10:377-393). Although familial PD (about 5% of the total cases) has been important to unveil the molecular pathways involved in neurodegeneration, most of the PD patients are sporadic (Obeso et al. (2017) Movement Disorders 32:1264-1310). Currently, no treatments are available that can slow or arrest neurodegeneration. The DA precursor L-DOPA is the gold standard for symptomatic treatments, although over time it may lead to several side effects and resistance (Nagatsua et al. (2009) Parkinsonism Related Disorders 15 (Suppl 1):S3-S8).

Glial cell-derived neurotrophic factor (GDNF) is the most potent neurotrophic factor for DA neurons with great potentiality for clinical application (Kordower et al. (2013) Movement Disorders 28:96-109 and Lin et al. (1993) Science 260:1130-1132). GDNF can protect DA cells and promotes their survival from toxic insults in vitro and in vivo in mice, rats, and non-human primates (Tomac et al. (1005) Nature 373:33-339, Gash et al. (1996) Nature 380:252-255 and Kirik et al. (2000) Eur. J. of Neuroscience 12:3871-3882). However, clinical trials with recombinant GDNF showed an inconsistency of results, due to the poor spreading of GDNF within the parenchyma and to the unsustainable side effects caused by the high doses of GDNF administered (Nutt et al. (2003) Neurology 60:69-73, Gill et al. (2003) Nat. Med. 9:589-595 and Lang et al. (2006) Ann. Neurol. 59:459-466). Similarly, GDNF expression through viral vectors that showed efficacy in PD animal models may result in uncontrolled, ectopic GDNF overexpression. Therefore, GDNF may be the right candidate for PD therapy, provided that a more specific and physiological expression is achieved to increase its efficacy and safety profile in vivo. In this context, a deletion in the 3′ UTR of the GDNF gene in a transgenic mouse line led to an increase of 2-fold of endogenous GDNF protein levels (Kumar et al. (2015) PLOS Genet. 11:e1005710). This was sufficient to induce alterations in the DA system and neuroprotection similar to that seen with a large overexpression of GDNF, but without the side effects. These data corroborate the hypothesis that a moderate increase in endogenous GDNF could be beneficial for PD.

cRET (also known as RET) is a tyrosine kinase receptor that is triggered by members of the GDNF family. RET conditional knock-out mice were shown to have late and progressive degeneration of dopaminergic (DA) neurons (Kramer et al. (2007) PLOS Biol. 5:e39), and degeneration of midbrain DA neurons has been indicated to be a key histopathological feature of PD. Furthermore, results from studies involving mutations disrupting the RET51/FKBP52 molecular complex have been suggestive of a possible role for this complex in DA neuron function and PD (Fusco et al. (2010) Hum. Mol. Genet. 19(14):2804-2816).

Orphan nuclear receptor Nurr1 (also known as NR4A2) has also been shown to be involved in the differentiation of midbrain DA neurons. Emerging evidence indicates that impaired Nurr1 function may contribute to the pathogenesis of PD (Decressac et al. (2013) Nat. Rev. Neurol. 9: 629-636).

A new class of long non-coding RNAs (lncRNAs), known as SINEUPs, were previously described to be able to selectively enhance their targets' translation. SINEUP activity relies on the combination of two domains: the overlapping region, or binding domain (BD), that confers specificity, and an embedded inverted SINE B2 element, or effector domain (ED), enhancing target mRNA translation. WO 2012/133947 and WO 2019/150346 disclose functional nucleic acid molecules including SINEUPs, and are incorporated herein by reference in their entirety. Another class of lncRNAs that use effector domains comprising an internal ribosome entry site (IRES) sequence to provide trans-acting functional nucleic acid molecules are described in WO 2019/058304.

The aim of the invention is to provide the first gene-specific technology targeting endogenous Nurr1, GDNF, cRET and GBA translation, particularly for use in treating diseases or disorders of the nervous system, such as neurodegenerative disorders.

### SUMMARY OF THE INVENTION

According to a first aspect of the invention, there is provided a therapeutic agent for use in a method of treating a disease or disorder of the nervous system, wherein the therapeutic agent increases translation of an endogenous mRNA sequence selected from the group consisting of: an endogenous Nurr1, human GDNF, cRET or GBA mRNA sequence.

According to a second aspect, there is provided a functional nucleic acid molecule comprising:
at least one target binding sequence comprising a sequence reverse complementary to a target mRNA sequence selected from the group consisting of: a Nurr1, GDNF, cRET or GBA mRNA sequence; and
at least one regulatory sequence comprising a SINE B2 element, a functionally active fragment of a SINE B2 element, an internal ribosome entry site (IRES) sequence or a functionally active fragment of an internal ribosome entry site (IRES) sequence.

According to a further aspect of the invention, there is provided a DNA molecule encoding the functional nucleic acid molecule as defined herein.

According to a yet further aspect, there is provided an expression vector comprising the functional nucleic acid molecule or the DNA molecule as defined herein.

According to a further aspect of the invention, there is provided a composition comprising the functional nucleic acid molecule, the DNA molecule or the expression vector as defined herein.

According to a further aspect of the invention, there is provided a pharmaceutical composition comprising the functional nucleic acid molecule, the DNA molecule or the expression vector as defined herein, in admixture with a suitable pharmaceutical excipient, diluent or carrier.

According to another aspect of the invention, there is provided a method for increasing the protein synthesis efficiency of Nurr1, GDNF, cRET or GBA in a cell comprising administering the functional nucleic acid molecule, the DNA molecule, the expression vector, the composition, or the pharmaceutical composition as defined herein, to the cell.

According to a further aspect, there is provided the functional nucleic acid molecule, the DNA molecule, the composition, or the pharmaceutical composition as defined herein, for use as a medicament.

According to a yet further aspect of the invention, there is provided the functional nucleic acid molecule, the DNA molecule, the composition, or the pharmaceutical composition as defined herein, for use in a method of treating a disease or disorder of the nervous system.

According to a further aspect, there is provided a method of treating a disease or disorder of the nervous system comprising administering a therapeutic agent that increases translation of an endogenous mRNA sequence selected from the group consisting of: an endogenous Nurr1, human GDNF, cRET or GBA mRNA sequence to a subject with a disease or disorder of the nervous system.

According to a still further aspect, there is provided a method of treating a disease or disorder of the nervous system comprising administering a therapeutically effective amount of the functional nucleic acid molecule, the DNA molecule, the composition, or the pharmaceutical composition as defined herein to a subject with a disease or disorder of the nervous system.

According to a further aspect, there is provided a method of producing a non-human animal model of Parkinson's disease comprising: administering a functional nucleic acid molecule comprising: at least one target binding sequence comprising a sequence reverse complementary to a SNCA mRNA sequence; and at least one regulatory sequence comprising a SINE B2 element, a functionally active fragment of a SINE B2 element, an IRES sequence or a functionally active fragment of an IRES sequence, to a non-human animal.

### BRIEF DESCRIPTION OF THE FIGURES

FIG. 1: Schematic representation of SINEUP functional domains. The overlap is the Binding Domain (BD, light grey) that provides SINEUP specificity and is in antisense orientation to the sense protein-coding mRNA (Target mRNA). The inverted SINE B2 (invB2, dark grey) element from AS Uchl1 is the Effector Domain (ED) and confers enhancement of protein synthesis. 5′ to 3′ orientation of sense and antisense RNA molecules is indicated. Structural elements of target mRNA are shown: 5′ untranslated region (5′UTR, white), coding sequence (CDS, black) and 3′ untranslated region (3′UTR, white). Scheme is not drawn in scale.

FIG. 2: Activity of functional nucleic acids targeting hGDNF. As determined by RT-qPCR, the SINEUPs were abundantly expressed in the HEK-GDNF-nLuc cell line. Expression of microSINEUPs (FIG. 2a). HEK-GDNF-nLuc cells expressing microSINEUPs displayed elevated levels of secreted nLuc, when compared to EV alone (FIG. 2b). The SINEUP BD-C elevated the level of luciferase compared to cells transfected with EV (FIG. 2c). HEK-GDNF-nLuc cells expressing miniSINEUPs also displayed elevated levels of secreted nLuc, when compared to EV alone (FIG. 2d). Expression of miniSINEUPs in HEK-GDNF-nLuc does not affect target GDNF mRNA expression (FIG. 2e).

FIG. 3: Activity of functional nucleic acids targeting hGDNF. Functional nucleic acids targeting human GDNF (hGDNF) with the following BDs were tested for their ability to increase protein expression of endogenous hGDNF in Hela cells: BDI: −14/−1 (methionine 1; M1); BDII: −14/+4 (methionine 1; M1); BDIII: −15/+4 (methionine 1; M1); BDIV: −14/+4 (methionine 2; M2); BDV: −25/−1 (methionine 2; M2); BDVI: −42/+4 (methionine 3; M3). GDNF BD1-BDVI increased GDNF protein levels. 48 hours post-transfection, hGDNF protein levels were increased as compared to an empty vector (−) (FIG. 3a). Expression of the functional nucleic acids did not affect hGDNF mRNA levels, as expected for a post-transcriptional mechanism (FIGS. 3b and 3c).

FIG. 4: Activity of functional nucleic acids targeting hcRET. Hela cells expressing microSINEUPs for 48 hrs displayed elevated levels of hcRET, when compared to empty vector alone (FIG. 4a). As determined by RT-qPCR, microSINEUPs targeted to hcRET were expressed in the HeLa cell line (FIG. 4b). Expression of microSINEUPs in Hela cells does not affect target hcRET mRNA expression (FIG. 4c).

FIG. 5: Activity of functional nucleic acids targeting hGBA. Hela cells expressing microSINEUPs for 48 hrs displayed elevated levels of hGBA, when compared to EV alone (FIG. 5a). As determined by RT-qPCR, microSINEUPs to hGBA were expressed in the HeLa cell line (FIG. 5b). Expression of microSINEUPs in Hela cells does not affect target hGBA mRNA expression (FIG. 5c).

FIG. 6: Activity of functional nucleic acids targeting haSYN. Hela cells expressing specific haSYN microSINEUPs for 48 hrs displayed elevated levels of haSYN, when compared to EV alone (FIG. 6a). As determined by RT-qPCR, microSINEUP BD8 to haSYN was expressed in the HeLa cell line (FIG. 6b). Expression of microSINEUPs in Hela cells does not affect target haSYN mRNA expression (FIG. 6c).

### DETAILED DESCRIPTION

It is an object of the present invention to provide novel methods of treating a disease or disorder of the nervous system by using a therapeutic agent that increases translation of an endogenous mRNA sequence selected from the group consisting of: an endogenous Nurr1, GDNF, cRET or GBA mRNA sequence. Current therapeutic methods that aim to target such diseases, do so by “replacement” or “supplement” therapy, for example by either delivering the target itself as a protein or by providing the gene (cDNA or mRNA) to encode the therapeutic protein. However, the methods and agents described herein are different because they target the endogenous mRNA sequences encoding therapeutically beneficial proteins already present in the cell (albeit at a reduced level) and modulate, such as increase, the expression of the endogenous protein or isoform thereof in order to ameliorate the disease. In particular, the invention provides functional nucleic acid molecules that increase the level of Nurr1, GDNF, CRET, GBA or SNCA protein expression without overcoming physiological levels. Furthermore, this technology targets endogenous mRNA sequences in a highly gene-specific manner and is only capable of driving expression in cells at sites where the endogenous natural product is expressed, thereby limiting side effects. To this end, the inventors have utilised SINEUP technology to develop and target Nurr1, GDNF, cRET, GBA or SNCA mRNA sequences in order to increase the endogenous levels of all Nurr1, GDNF, CRET, GBA or SNCA proteins in human and mouse.

### Definitions

By “functional nucleic acid molecule” there is intended generally that the nucleic acid molecule (e.g. DNA or RNA) is capable of enhancing the translation of a target mRNA of interest, in this particular case a Nurr1, GDNF, cRET, GBA or SNCA mRNA. Such molecules may be referred to as “trans-acting functional nucleic acid molecules” because they promote gene-specific translation up-regulation and act on endogenous mRNAs.

By “endogenous mRNA sequence” there is intended an mRNA sequence of any length (preferably at least 10 nucleotides) that is already present in the target cell. The endogenous mRNA sequence is encoded by a target gene, wherein the target gene is preferably selected from the list comprising: the NR4A2, GDNF, RET, GBA or SNCA gene. Alternative splicing of the endogenous transcripts leads to the generation of various isoforms as described herein.

Alternative splicing of the GDNF mRNA produces 5 protein coding isoforms (UniProt and NCBI database browsers), 3 of which share at least a portion of the 5′ UTR. The isoforms are expressed differentially in different tissues, however all isoforms are expressed in the brain with expression predominantly in the striatum and the highest levels are found in the caudate and lowest in the putamen. The GDNF gene sequence is known in the art, for example see Ensembl ID: ENSG00000168621. The GDNF gene encodes glial cell derived neurotrophic factor (also known as astrocyte-derived trophic factor (ATF)) and is referred to herein as “GDNF protein”. The GDNF protein sequence is known in the art, for example see UniProt ID: P39905. GDNF target binding sequences disclosed herein were designed on the isoform NM_000514.4 but it will be readily appreciated that such target binding sequences will bind any isoform or spice variant comprising the sequence to which the target binding sequences bind, such as NM_199231, NM_001190468.1 and NM_001190469.1 which share the 5′ UTR with NM_000514.4.

Alternative splicing of the cRET mRNA (also known as RET) produces 3 protein coding isoforms (NCBI database browser) which share a 5′ UTR. Of particular interest herein is the long isoform of RET known as RET51. The RET gene sequence is known in the art, for example see Ensembl ID: ENSG00000165731. The RET gene encodes proto-oncogene tyrosine-protein kinase receptor Ret (also known as cadherin family member 12 and proto-oncogene c-Ret) and is referred to herein as “cRET protein”. The cRET protein sequence is known in the art, for example see UniProt ID: P07949. RET target binding sequences disclosed herein were designed on the RET51 isoform but it will be readily appreciated that such target binding sequences will bind any isoform or spice variant comprising the sequence to which the target binding sequences bind, such as RET9 (NM_020630.6).

Alternative splicing of the Nurr1 (also known as NR4A2) mRNA produces 8 protein coding isoforms (Ensembl database browser), 4 of which share a portion of the 5′ UTR near the AUG start codon. They are expressed in several cell lines of T cell, B cell and fibroblast origin and show strong expression in brain tissue. The Nurr1 protein is encoded by the NR4A2 gene sequence which is known in the art, for example see Ensembl ID: ENSG00000153234. The NR4A2 gene encodes nuclear receptor subfamily 4 group A member 2 (also known as immediate-early response protein NOT, orphan nuclear receptor NURR 1 and transcriptionally-inducible nuclear receptor) and is referred to herein as “Nurr1 protein”. The Nurr1 protein sequence is known in the art, for example see UniProt ID: P43354. Nurr1 target binding sequences disclosed herein were designed on the isoform NM_006186.4 but it will be readily appreciated that such target binding sequences will bind any isoform or spice variant comprising the sequence to which the target binding sequences bind.

Alternative splicing of the GBA mRNA produces 5 isoforms (UniProt and NCBI database browsers). The GBA gene sequence is known in the art, for example see Ensembl ID: ENSG00000177628. The GBA gene encodes glucosylceramidase beta (also known as β-glucocerebrosidase) and is referred to herein as “GBA protein”. The GBA protein sequence is known in the art, for example see UniProt ID: P04062. GBA target binding sequences disclosed herein were designed on the isoform NM_000157.4 but it will be readily appreciated that such target binding sequences will bind any isoform or spice variant comprising the sequence to which the target binding sequences bind, such as NM_001171811.2, NM_001005741.3, NM_001005742.3 and NM_001171812.2 which share a region of the 5′ UTR 49 bp upstream of M1 compared to NM_000157.4.

Alternative splicing of the SNCA mRNA produces 3 isoforms (UniProt database browser). The SNCA gene sequence is known in the art, for example see Ensembl ID: ENSG00000145335. The SNCA gene encodes α-synuclein (also known as alpha-synuclein or synuclein alpha) and may also be referred to herein as “SNCA protein”. Alpha synuclein may also be abbreviated to αSYN, that is the mRNA or protein may be referred to as simply Alpha synuclein (αSYN) or human (hαSYN). The SNCA protein sequence is known in the art, for example see UniProt ID: P37840. SNCA target binding sequences disclosed herein were designed on the isoform NM_000345.4 but it will be readily appreciated that such target binding sequences will bind any isoform or spice variant comprising the sequence to which the target binding sequences bind, such as NM_007308.3 which lacks an alternate in-frame exon compared to NM_000345.4.

The term “SINE” (Short Interspersed Nuclear Element) may be referred to as a non-LTR (long terminal repeat) retrotransposon, and is an interspersed repetitive sequence (a) which encodes a protein having neither reverse-transcription activity nor endonuclease activity or the like and (b) whose complete or incomplete copy sequences exist abundantly in genomes of living organisms.

The term “SINE B2 element” is defined in WO 2012/133947, which is incorporated herein by reference in its entirety, where specific examples are also provided (see table starting on page 69 of the PCT publication). The term is intended to encompass both SINE B2 elements in direct orientation and in inverted orientation relative to the 5′ to 3′ orientation of the functional nucleic acid molecule. SINE B2 elements may be identified, for example, using programs like RepeatMask as published (Bedell et al. (2000) Bioinformatics 16(11): 1040-1. MaskerAid: a performance enhancement to RepeatMasker). A sequence may be recognizable as a SINE B2 element by returning a hit in a Repbase database with respect to a consensus sequence of a SINE B2, with a Smith-Waterman (SW) score of over 225, which is the default cutoff in the RepeatMasker program. Generally a SINE B2 element is not less than 20 bp and not more than 400 bp. Preferably, the SINE B2 is derived from tRNA.

By the term “functionally active fragment of a SINE B2 element” there is intended a portion of sequence of a SINE B2 element that retains protein translation enhancing efficiency or protein translation enhancing activity. This term also includes sequences which are mutated in one or more nucleotides with respect to the wild-type sequences, but retain protein translation enhancing efficiency. The term is intended to encompass both SINE B2 elements in direct orientation and in inverted orientation relative to the 5′ to 3′ orientation of the functional nucleic acid molecule.

The terms “internal ribosome entry site (IRES) sequence” and “internal ribosome entry site (IRES) derived sequence” are defined in WO 2019/058304, which is incorporated herein by reference in its entirety. IRES sequences recruit the 40S ribosomal subunit and promote cap-independent translation of a subset of protein coding mRNAs. IRES sequences are generally found in the 5′ untranslated region of cellular mRNAs coding for stress-response genes, thus stimulating their translation in cis. It will be understood by the term “functionally active fragment of an IRES sequence” there is intended a portion of sequence of an “IRES sequence” that retains protein translation enhancing activity, fragments may be of varying lengths as described herein. It will be understood by the term “IRES derived sequence” there is intended a sequence of nucleic acid with a homology to an IRES sequence so as to retain the functional activity thereof, i.e. a translation enhancing activity. In particular, the IRES derived sequence can be obtained from a naturally occurring IRES sequence by genetic engineering or chemical modification, e.g. by isolating a specific sequence of the IRES sequence which remains functional, or mutating/deleting/introducing one or more nucleotides in the IRES sequence, or replacing one or more nucleotides in the IRES sequence with structurally modified nucleotides or analogs. More particularly, the person skilled in the art would know that an IRES derived sequence, or a functionally active fragment of an IRES sequence, is a nucleotide sequence capable of promoting translation of a second cistron in a bicistronic construct. Typically, a dual luciferase (Firefly luciferase, Renilla Luciferase) encoding plasmid is used for experimental tests, such as functional experimental tests. A major database exists, namely IRESite, for the annotation of nucleotide sequences that have been experimentally validated as IRES, using dual reporter or bicistronic assays (http://iresite.org/IRESite_web.php). Within IRESite, a web-based tool is available to search for sequence-based and structure-based similarities between a query sequence of interest and the entirety of annotated and experimentally validated IRES sequences within the database. The output of the program is a probability score for any nucleotide sequence to be able to act as IRES in a validation experiment with bicistronic constructs. Additional sequence-based and structure-based web-based browsing tools are available to suggest, with a numerical predicting value, the IRES activity potentials of any given nucleotide sequence (http://rna.informatik.uni-freiburg.de/ and http://regrna.mbc.nctu.edu.tw/index1.php).

By the term “miniSINEUP” there is intended a nucleic acid molecule comprising (or consisting of) a binding domain (i.e. a complementary sequence to target mRNA), optionally a spacer sequence, and any SINE or SINE-derived sequence or IRES or IRES-derived sequence as the effector domain (Zucchelli et al. (2015) Front. Cell. Neurosci. 9:174).

By the term “microSINEUP” there is intended a nucleic acid molecule comprising (or consisting of) a binding domain (i.e. a complementary sequence to target mRNA), optionally a spacer sequence, and a functionally active fragment of the SINE or SINE-derived sequence, IRES sequence or IRES-derived sequence. For example, the functionally active fragment may be a 77 bp sequence corresponding to nucleotides 44 to 120 of the 167 bp SINE B2 element in AS Uchl1.

miniSINEUP and microSINEUP are further defined in WO 2019/150346 and PCT/GB2021/052502, which are incorporated herein by reference in their entirety

Polypeptide or polynucleotide sequences are said to be the same as or “identical” to other polypeptide or polynucleotide sequences, if they share 100% sequence identity over their entire length. Residues in sequences are numbered from left to right, i.e. from N- to C-terminus for polypeptides; from 5′ to 3′ terminus for polynucleotides. If closely related sequences are not identical they may be similar, i.e., they may possess a certain, quantifiable, degree of sequence identity, e.g., a sequence may have 50%, 60%, 70%, 80%, 90%, 95% or 99% sequence identity to another sequence. Unless a specific reference range is given, e.g., with respect to the nucleotide positions, any quoted sequence identity will be understood as being calculated across the residue range over which the two sequences are aligned. The aligned residue range may represent the entirety of one or more of the input sequences or a contiguous section of sequence of one or more of the input sequences, and is typically determined by standard tools known in the art, e.g., NCBI BLAST.

For the purposes of comparing two closely-related polynucleotide sequences, the “% sequence identity” between a first nucleotide sequence and a second nucleotide sequence may be calculated using NCBI BLAST, using standard settings for nucleotide sequences (BLASTN). For the purposes of comparing two closely-related polypeptide sequences, the “% sequence identity” between a first polypeptide sequence and a second polypeptide sequence may be calculated using NCBI BLAST, using standard settings for polypeptide sequences (BLASTP). A “difference” between sequences refers to an insertion, deletion or substitution of a single nucleotide in a position of the second sequence, compared to the first sequence. Two sequences can contain one, two or more such differences. Insertions, deletions or substitutions in a second sequence which is otherwise identical (100% sequence identity) to a first sequence result in reduced % sequence identity.

References herein to “a disease or disorder of the nervous system” may include diseases of the central nervous system, in particular neurodegenerative disorders. This term may be used generally herein to refer to any disease or disorder that affects the functioning of the nervous system. Nervous system disorders include disorders of the central nervous system (i.e. brain and spinal cord), peripheral nervous system (i.e. all other neural elements, including the peripheral nerves and the autonomic nerves), and mental health and psychiatric disorders. The disorder may be a neurodegenerative disorder (e.g. a disorder associated with the degeneration of neurons). Such disorders include Parkinson's disease, multiple sclerosis, amyotrophic lateral sclerosis (ALS), Huntington's disease, and Alzheimer's disease. The actual symptoms associated with a nervous system disorder disclosed herein are well known and can be determined by a person of ordinary skill in the art by accounting for factors, including, the location, cause and severity of the nervous system disorder. Examples of symptoms can be wide-ranging but may include inflammation, fatigue, dizziness, headache, malaise, elevated fever and high body temperature, weakness and stiffness in muscles and joints, weight changes, digestive or gastrointestinal problems, low or high blood pressure, irritability, anxiety, depression, blurred, impaired or double vision, ataxia, paralysis, impaired muscle coordination, loss of sensation and speech problems. Treatment using the functional nucleic acid molecules described herein may reduce one or more of these symptoms.

### Therapeutic Agents and Functional Nucleic Acid Molecules

According to a first aspect of the invention, there is provided a therapeutic agent for use in a method of treating a disease or disorder of the nervous system, wherein the therapeutic agent increases translation of an endogenous mRNA sequence selected from the group consisting of: an endogenous Nurr1, human GDNF, cRET or GBA mRNA sequence.

In one embodiment, there is provided a therapeutic agent for use in a method of treating a disease or disorder of the central nervous system.

In one embodiment, there is provided a therapeutic agent for use in a method of treating a disease or disorder of the central nervous system, wherein the disease or disorder is a neurodegenerative disease.

In one embodiment, there is provided a therapeutic agent for use in a method of treating a disease or disorder of the central nervous system, wherein the disease or disorder is Parkinson's disease.

In one embodiment, there is provided a therapeutic agent for use in a method of treating a disease or disorder of the central nervous system, wherein the therapeutic agent comprises a functional nucleic acid molecule comprising:
at least one target binding sequence comprising a sequence reverse complementary to a target mRNA sequence selected from the group consisting of: a Nurr1, GDNF, cRET or GBA mRNA sequence; and
at least one regulatory sequence comprising a SINE B2 element, a functionally active fragment of a SINE B2 element, an internal ribosome entry site (IRES) sequence or a functionally active fragment of an IRES sequence.

According to one aspect of the present invention, there is provided a functional nucleic acid molecule comprising:
at least one target binding sequence comprising a sequence reverse complementary to a target mRNA sequence selected from the group consisting of: a Nurr1, GDNF, cRET or GBA mRNA sequence; and
at least one regulatory sequence comprising a SINE B2 element, a functionally active fragment of a SINE B2 element, an internal ribosome entry site (IRES) sequence or a functionally active fragment of an IRES sequence.

According to one aspect of the present invention, there is provided a functional nucleic acid molecule comprising:
at least one target binding sequence comprising a sequence reverse complementary to a target mRNA sequence selected from the group consisting of: a Nurr1, human GDNF, CRET or GBA mRNA sequence; and
at least one regulatory sequence comprising an RNA comprising a SINE B2 element, a functionally active fragment of a SINE B2 element, an internal ribosome entry site (IRES) sequence or a functionally active fragment of an IRES sequence.

As used herein, “therapeutic agent” refers to an agent which may be used to treat a disease or disorder in said subject (such as in a cell of a subject). The therapeutic agent may treat said disease or disorder by enhancing translation of target proteins, such as enhancing translation of target mRNA by using a functional nucleic acid molecule according to the present invention.

As described herein, the listed targets have been indicated to be involved in the pathology of nervous system diseases, such as neurodegenerative diseases. In one embodiment, the target mRNA sequence is selected from the group consisting of: a Nurr1, cRET or GBA mRNA sequence. In one embodiment, the target mRNA sequence is a GBA mRNA sequence. In one embodiment, the target mRNA sequence is a human GDNF mRNA sequence. In one embodiment, the target mRNA sequence is a cRET mRNA sequence. In one embodiment, the target mRNA sequence is a Nurr1 mRNA sequence.

### Regulatory Sequences

The regulatory sequence has protein translation enhancing efficiency. In some embodiments the regulatory sequence has protein translation enhancing activity. The increase of the protein translation efficiency indicates that the efficiency is increased as compared to a case where the functional nucleic acid molecule according to the present invention is not present in a system. In one embodiment, expression of the protein encoded by the target mRNA is increased by at least 1.2 fold, by at least 1.3 fold, by at least 1.4 fold, by at least 1.5 fold, by at least 1.6 fold, by at least 1.7 fold, by at least 1.8 fold, by at least 1.9 fold, in particular by at least 2 fold. In a further embodiment, expression of the protein encoded by the target mRNA is increased between 1.5 to 3 fold, such as between 1.6 and 2.2 fold. It is envisaged that increasing protein expression within these ranges will allow the treatment of diseases or disorders of the nervous system without leading to negative side-effects associated with increasing expression of the target above physiological levels.

In one embodiment, the regulatory sequence is located 3′ of the target binding sequence. The regulatory sequence may be in a direct or inverted orientation relative to the 5′ to 3′ orientation of the functional nucleic acid molecule. Reference to “direct” refers to the situation in which the regulatory sequence is embedded (inserted) with the same 5′ to 3′ orientation as the functional nucleic acid molecule. Instead, “inverted” refers to the situation in which the regulatory sequence is 3′ to 5′ oriented relative to the functional nucleic acid molecule.

Preferably, the at least one regulatory sequence comprises a sequence with at least 75% sequence identity, at least 80% sequence identity, at least 85% sequence identity, at least 90% sequence identity, at least 91% sequence identity, at least 92% sequence identity, at least 93% sequence identity, at least 94% sequence identity, preferably at least 95% sequence identity, at least 96% sequence identity, at least 97% sequence identity, at least 98% sequence identity, at least 99% sequence identity, more preferably 100% sequence identity with a sequence selected from the group consisting of SEQ ID NOs: 1-69. In one embodiment, the at least one regulatory sequence consists of a sequence with at least 75% sequence identity, at least 80% sequence identity, at least 85% sequence identity, at least 90% sequence identity, at least 91% sequence identity, at least 92% sequence identity, at least 93% sequence identity, at least 94% sequence identity, preferably at least 95% sequence identity, at least 96% sequence identity, at least 97% sequence identity, at least 98% sequence identity, at least 99% sequence identity, more preferably 100% sequence identity with a sequence selected from the group consisting of SEQ ID NOs: 1-69.

In one embodiment, there is provided a therapeutic agent or functional nucleic acid molecule according to the invention, wherein the at least one regulatory sequence comprises a sequence with at least 75% sequence identity with a sequence selected from the group consisting of SEQ ID NOs: 1-69.

In one embodiment, there is provided a therapeutic agent or functional nucleic acid molecule according to the invention, wherein the at least one regulatory sequence comprises a sequence with at least 80% sequence identity with a sequence selected from the group consisting of SEQ ID NOs: 1-69.

In one embodiment, there is provided a therapeutic agent or functional nucleic acid molecule according to the invention, wherein the at least one regulatory sequence comprises a sequence with at least 85% sequence identity with a sequence selected from the group consisting of SEQ ID NOs: 1-69.

In one embodiment, there is provided therapeutic agent or functional nucleic acid molecule according to the invention, wherein the at least one regulatory sequence comprises a sequence with at least 90% sequence identity with a sequence selected from the group consisting of SEQ ID NOs: 1-69.

In one embodiment, there is provided a therapeutic agent or functional nucleic acid molecule according to the invention, wherein the at least one regulatory sequence comprises a sequence with at least 95% sequence identity with a sequence selected from the group consisting of SEQ ID NOs: 1-69.

In one embodiment, there is provided a therapeutic agent or functional nucleic acid molecule according to the invention, wherein the at least one regulatory sequence consists of a sequence with at least 75% sequence identity with a sequence selected from the group consisting of SEQ ID NOs: 1-69.

In one embodiment, there is provided a therapeutic agent or functional nucleic acid molecule according to the invention, wherein the at least one regulatory sequence consists of a sequence with at least 80% sequence identity with a sequence selected from the group consisting of SEQ ID NOs: 1-69.

In one embodiment, there is provided a therapeutic agent or functional nucleic acid molecule according to the invention, wherein the at least one regulatory sequence consists of a sequence with at least 85% sequence identity with a sequence selected from the group consisting of SEQ ID NOs: 1-69.

In one embodiment, there is provided a therapeutic agent or functional nucleic acid molecule according to the invention, wherein the at least one regulatory sequence consists of a sequence with at least 90% sequence identity with a sequence selected from the group consisting of SEQ ID NOs: 1-69.

In one embodiment, there is provided a therapeutic agent or functional nucleic acid molecule according to the invention, wherein the at least one regulatory sequence consists of a sequence with at least 95% sequence identity with a sequence selected from the group consisting of SEQ ID NOs: 1-69.

In one embodiment, the regulatory sequence comprises a SINE B2 element or a functionally active fragment of a SINE B2 element. The SINE B2 element is preferably in an inverted orientation relative to the 5′ to 3′ orientation of the functional nucleic acid molecule, i.e. an inverted SINE B2 element. As mentioned in the definitions section, inverted SINE B2 elements are disclosed and exemplified in WO 2012/133947, which is incorporated herein by reference in its entirety.

In one embodiment, the regulatory sequence consists of a SINE B2 element or a functionally active fragment of a SINE B2 element.

In one embodiment, the at least one regulatory sequence comprises a sequence with at least 75% sequence identity, at least 80% sequence identity, at least 85% sequence identity, at least 90% sequence identity, at least 91% sequence identity, at least 92% sequence identity, at least 93% sequence identity, at least 94% sequence identity, preferably at least 95% sequence identity, at least 96% sequence identity, at least 97% sequence identity, at least 98% sequence identity, at least 99% sequence identity, more preferably 100% sequence identity with a sequence selected from the group consisting of SEQ ID NOs: 1-51.

In one embodiment, the at least one regulatory sequence consists of a sequence with at least 75% sequence identity, at least 80% sequence identity, at least 85% sequence identity, at least 90% sequence identity, at least 91% sequence identity, at least 92% sequence identity, at least 93% sequence identity, at least 94% sequence identity, preferably at least 95% sequence identity, at least 96% sequence identity, at least 97% sequence identity, at least 98% sequence identity, at least 99% sequence identity, more preferably 100% sequence identity with a sequence selected from the group consisting of SEQ ID NOs: 1-51.

SEQ ID NO: 1 (the 167 nucleotide variant of the inverted SINE B2 element in AS Uchl1) and SEQ ID NO: 2 (the 77 nucleotide variant of the inverted SINE B2 element in AS Uchl1 that includes nucleotides 44 to 120), as well as sequences with percentage identity to these sequences, are particularly preferred.

Other inverted SINE B2 elements and functionally active fragments of inverted SINE B2 elements are SEQ ID NO: 3-51. Experimental data showing the protein translation enhancing efficiency of these sequences is not explicitly shown in the present patent application, but is disclosed in e.g., WO 2019/150346, which is incorporated herein by reference in its entirety. SEQ ID NOs: 3-51 can therefore also be used as regulatory sequences in molecules according to the present invention.

SEQ ID NOs: 3-6, 8-11, 18, 43-51 are functionally active fragments of inverted SINE B2 transposable element derived from AS Uchl1. The use of functional fragments reduces the size of the regulatory sequence which is advantageous if used in an expression vector (e.g. viral vectors which may be size-limited) because this provides more space for the target sequence and/or expression elements.

SEQ ID NO: 7 is a full length 183 nucleotide inverted SINE B2 transposable element derived from AS Uchl1. SEQ ID NOs: 12-17, 19, 20, 39-42 are mutated functionally active fragments of inverted SINE B2 transposable element derived from AS Uchl1.

SEQ ID NOs: 21-25, 28-38 are different SINE B2 transposable elements. SEQ ID NOs: 26 and 27 are sequences in which multiple inverted SINE B2 transposable element have been inserted.

Short fragments of the regulatory sequence (such as a SINE B2 element) are particularly useful when providing functional RNA molecules for use as a nucleic acid therapeutic. RNA molecules are highly unstable in living organisms, therefore stability provided by the chemical modifications as described herein, is more effective for shorter RNA molecules. Therefore, in one embodiment, the regulatory sequence comprises a functionally active fragment that is less than 250 nucleotides, such as less than 240 nucleotides, less than 230 nucleotides, less than 220 nucleotides, less than 210 nucleotides, less than 200 nucleotides, less than 190 nucleotides, less than 180 nucleotides, less than 170 nucleotides, less than 160 nucleotides, less than 150 nucleotides, less than 140 nucleotides, less than 130 nucleotides, less than 120 nucleotides, less than 110 nucleotides, less than 100 nucleotides, less than 90 nucleotides, less than 80 nucleotides, less than 70 nucleotides, less than 60 nucleotides, less than 50 nucleotides, less than 40 nucleotides, less than 30 nucleotides, less than 20 nucleotides, less than 10 nucleotides.

In some embodiments the regulatory sequence comprises or consists of a functionally active fragment of a sequence selected from the group consisting of SEQ ID NOs 2-52, wherein the functionally active fragment is about is about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250 or more nucleotides in length.

In one embodiment, the regulatory sequence comprises a SINE B2 element, wherein said SINE B2 element comprises a sequence selected from the group consisting of SEQ ID NOS 1-51, or a functionally active fragment thereof.

In one embodiment, the regulatory sequence acid molecule comprises a SINE B2 element, wherein said SINE B2 element consists of a sequence selected from the group consisting of SEQ ID NOs 1-51, or a functionally active fragment thereof.

Alternatively, the regulatory sequence comprises an IRES sequence, an IRES derived sequence, or a functionally active fragment of an IRES sequence. Therefore, in one embodiment, the regulatory sequence comprises an IRES sequence or, IRES derived sequence, or a functionally active fragment of an IRES sequence. Said sequence enhances translation of the target mRNA sequence.

Several IRESs having sequences ranging from 48 to 576 nucleotides have been tested with success, e.g. human Hepatitis C Virus (HCV) IRESs (e.g. SEQ ID NOs: 52 and 53), human poliovirus IRESs (e.g. SEQ ID NOs: 54 and 55), human encephalomyocarditis (EMCV) virus (e.g. SEQ ID NOs: 56 and 57), human cricket paralysis (CrPV) virus (e.g. SEQ ID NOs: 58 and 59), human Apaf-1 (e.g. SEQ ID NOs: 60 and 61), human ELG-1 (e.g. SEQ ID NOs: 62 and 63), human c-MYC (e.g. SEQ ID NOs: 64-67), human dystrophin (DMD) (e.g. SEQ ID NOs: 68 and 69).

Such sequences have been disclosed, defined and exemplified in WO 2019/058304, which is incorporated herein by reference in its entirety. Preferably, such sequences have at least 75%, at least 80% sequence identity, at least 85% sequence identity, at least 90% sequence identity, at least 91% sequence identity, at least 92% sequence identity, at least 93% sequence identity, at least 94% sequence identity, preferably at least 95% sequence identity, at least 96% sequence identity, at least 97% sequence identity, at least 98% sequence identity, at least 99% sequence identity, more preferably 100% sequence identity to any of SEQ ID NOs: 52-69.

In one embodiment, the at least one regulatory sequence comprises a sequence selected from the group consisting of SEQ ID NOs 52-69, or a functionally active fragment thereof.

In one embodiment, the at least one regulatory sequence consists of a sequence selected from the group consisting of SEQ ID NOs 52-69, or a functionally active fragment thereof.

In one embodiment, the at least one regulatory sequence comprises a sequence with at least 75% sequence identity, at least 80% sequence identity, at least 85% sequence identity, at least 90% sequence identity, at least 91% sequence identity, at least 92% sequence identity, at least 93% sequence identity, at least 94% sequence identity, preferably at least 95% sequence identity, at least 96% sequence identity, at least 97% sequence identity, at least 98% sequence identity, at least 99% sequence identity, more preferably 100% sequence identity with a sequence selected from the group consisting of SEQ ID NOs: 52-69.

In one embodiment, the at least one regulatory sequence consists of a sequence with at least 75% sequence identity, at least 80% sequence identity, at least 85% sequence identity, at least 90% sequence identity, at least 91% sequence identity, at least 92% sequence identity, at least 93% sequence identity, at least 94% sequence identity, preferably at least 95% sequence identity, at least 96% sequence identity, at least 97% sequence identity, at least 98% sequence identity, at least 99% sequence identity, more preferably 100% sequence identity with a sequence selected from the group consisting of SEQ ID NOs: 52-69.

In some embodiments the at least one regulatory sequence comprises or consists of a functionally active fragment of any one of SEQ ID NOs 53-70, wherein the functionally active fragment is about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370 or more nucleotides in length.

In some embodiments, the functionally active fragment retains IRES activity within the definition provided herein, i.e., within the The Standard Bicistronic Plasmid Test.

In some embodiments, the functionally active fragment retains protein translation enhancing activity.

It will be understood that, owing to the functional nature of functional experimental tests for IRES activity, e.g., The Standard Bicistronic Plasmid Test for Cellular mRNA IRESs, a “functionally active fragment” of an IRES might also be considered an IRES per se. Herein, “functionally active fragment” of an IRES is utilised to delineate IRES sequences that are shorter in length as compared with ‘parental’ IRES sequences from which they are designed or derived.

### Target Binding Sequences

In some embodiments, the at least one target binding sequence comprises a sequence reverse complementary to a target mRNA sequence selected from the group consisting of: human Nurr1, human GDNF, human cRET or human GBA mRNA sequence. In other embodiments, the at least one target binding sequence comprises a sequence reverse complementary to a target mRNA sequence selected from the group consisting of: mouse Nurr1, mouse cRET or mouse GBA mRNA sequence. In one embodiment, the at least one target binding sequence does not comprise a sequence reverse complementary to mouse GDNF.

Human GDNF is a highly conserved neurotropic factor which can promote the survival and differentiation of dopaminergic neurons in vitro and is able to prevent the axotomy-induced apoptosis of motor neurons. The encoded protein is processed to a mature secreted form that exists as a homodimer which is a ligand for the product of the RET (rearranged during transfection) proto-oncogene. In addition to the transcript encoding GDNF, two additional alternative transcripts encoding distinct proteins, referred to as astrocyte-derived trophic factors, have been described. GDNF has also been found to regulate kidney development and spermatogenesis, and it promotes hair follicle formation and cutaneous wound healing by targeting resident hair follicle stem cells (BSCs) in the bulge compartment.

Human cRET is encoded by the RET proto-oncogene and is a receptor tyrosine kinase for members of the glial cell line-derived neurotrophic factor (GDNF) family of extracellular signalling molecules. Loss of function mutations have been associated with the development of Hirschsprung's disease, while gain of function mutations are associated with the development of various types of human cancer, including medullary thyroid carcinoma, multiple endocrine neoplasia type 2A and type 2B, pheochromocytoma and parathyroid hyperplasia. The human RET gene is localized to chromosome 10 (10q11.2) and contains 21 exons which are alternatively spliced to produce 3 different isoforms of the protein. RET51, RET43 and RET9 contain 51, 43 and 9 amino acids in their C-terminal tail, respectively. RET51 is one of the most common isoforms produced from the RET gene. Common to each isoform is a domain structure, with each protein divided into three domains: an N-terminal extracellular domain with four cadherin-like repeats and a cysteine-rich region, a hydrophobic transmembrane domain and a cytoplasmic tyrosine kinase domain, which is split by an insertion of 27 amino acids. Within the cytoplasmic tyrosine kinase domain, there are 16 tyrosine residues (Tyr) in RET9 and 18 in RET51. Tyr1090 and Tyr1096 are present only in the RET51 isoform. Activating point mutations in cRET can give rise to the hereditary cancer syndrome known as multiple endocrine neoplasia type 2 (MEN 2) and there is a high degree of correlation between the position of the point mutation and the phenotype of the disease. Chromosomal rearrangements that generate a fusion gene, resulting in the juxtaposition of the C-terminal region of the RET protein with an N-terminal portion of another protein, can also lead to constitutive activation of the RET kinase. These types of rearrangements are primarily associated with papillary thyroid carcinoma (PTC) and non-small cell lung cancer (NSCLC). Several fusion partners have been described, and the most common ones across both cancer types include KIF5B, CCDC6 and NCOA4.

Nuclear receptor related 1 protein (Nurr1) also known as NR4A2 (nuclear receptor subfamily 4, group A, member 2) is a member of the nuclear receptor family of intracellular transcription factors and in humans is encoded by the NR4A2 gene. Nurr1 plays a key role in the maintenance of the dopaminergic system of the brain and mutations in this gene have been associated with disorders related to dopaminergic dysfunction, including Parkinson's disease and schizophrenia. Several transcript variants encoding distinct isoforms have been identified for this gene, although additional alternate splice variants may exist of which the full-length nature has not yet been determined. Nurr1 is believed to be critical to development of the dopamine phenotype in the midbrain, as knockout mice lack expression of this phenotype. This has been further confirmed by studies showing that when forcing Nurr1 expression in naïve precursor cells, there is complete dopamine phenotype gene expression. Nurr1 induces tyrosine hydroxylase (TH) expression, which causes differentiation into dopaminergic neurons, and Nurr1 has been shown to induce differentiation in central nervous system precursor cells in vitro. Therefore, Nurr1 modulation has been suggested for the generation of dopaminergic neurons for Parkinson's disease research. Nurr1 may also have a role in inflammation, in particular in inflammatory disorders caused by dopaminergic neuron disease. Inflammation in the central nervous system can result from activated microglia and other pro-inflammatory factors, such as bacterial lipopolysaccharide (LPS). LPS binds to toll-like receptors (TLR), which induces inflammatory gene expression by promoting signal-dependent transcription factors. It has been shown that Nurr1 protects dopaminergic neurons from LPS-induced inflammation, by reducing inflammatory gene expression in microglia and astrocytes. Furthermore, if a short hairpin for Nurr1 is expressed in microglia and astrocytes, these cells produce inflammatory mediators, such as TNFα, NO synthase and IL-1β, demonstrating that reduced Nurr1 promotes inflammation and leads to cell death of dopaminergic neurons. Nurr1 interacts with the transcription factor complex NF-κB-p65 on inflammatory gene promoters, but these interactions are dependent on other factors. Sumoylation of Nurr1 is required and its co-regulating factor, glycogen synthase kinase 3, needs to be phosphorylated for these interactions to occur. Sumolyated Nurr1 recruits CoREST, a complex made of several proteins that assembles chromatin-modifying enzymes, and the resulting Nurr1/CoREST complex inhibits the transcription of inflammatory genes.

GBA, acid beta-glucocerebrosidase, also known as beta-glucosidase, is a lysosomal enzyme that catalyzes the breakdown of the glycolipid glucosylceramide to ceramide and glucose. Alternative names include GBA1, acid beta-glucosidase, beta-glucosidase, beta-GC, glucocerebrosidase or glucosylceramidase. GBA mutations are thought to reduce the enzymatic function of the acid beta-glucocerebrosidase, impairing lysosomal efficiency and the cellular ability to dispose of pathological alpha-synuclein. Heterozygous mutations of the GBA gene are a major genetic risk factor for Parkinson's disease (PD) and dementia with Lewy bodies (DLB). Homozygous mutations in the GBA gene are also linked to Gaucher's disease (GD), a multi-organ disease that can be divided into at least four types, some associated with neuropathology. Enzyme replacement therapy (including Ceredase® or Cerezyme®) is a therapeutic option but does not address the neuropathic forms of the disease (see Deegan et al. Drug Des. Devel. Ther. (2012) 6: 81-106; Avenali et al. Front Aging Neurosci. (2020)12: 97; and Creese et al. Am. J. Med. Genet. B. Neuropsychiatr. Genet. (2018) 177(2): 232-241).

In one embodiment, the target binding sequence comprises a sequence reverse complementary to a portion of the Nurr1, GDNF, cRET or GBA mRNA sequence that is common to all Nurr1, GDNF, cRET or GBA isoforms. By maintaining the reciprocal levels of all Nurr1, GDNF, cRET or GBA isoforms, the functional nucleic acid molecule is able to induce the best molecular pattern of expression to restore physiological homeostasis. This is not possible with a more conventional gene therapy approaches when only one isoform can be ectopically expressed leading to isoform imbalance.

In WO 2012/133947, which is incorporated herein by reference in its entirety, it was already shown that the target binding sequence needs to have only about 60% similarity with a sequence reverse complementary to the target mRNA. As a matter of fact, the target binding sequence can even display a large number of mismatches and retain activity.

The target binding sequence comprises a sequence which is sufficient in length to bind to the Nurr1, GDNF, cRET or GBA mRNA transcript. Therefore, the target binding sequence may be at least 10 nucleotides long, such as at least 11 nucleotides long, such as at least 12 nucleotides long, such as at least 13 nucleotides long, such as least 14 nucleotides long, such as least 15 nucleotides long, such as at least 16 nucleotides long, such as at least 17 nucleotides long, such as least 18 nucleotides long, such as at least 19 nucleotides long, or such as at least 20 nucleotides long. Furthermore, the target binding sequence may be less than about 250 nucleotides long, preferably less than about 200 nucleotides long, less than about 150 nucleotides long, less than about 140 nucleotides long, less than about 130 nucleotides long, less than about 120 nucleotides long, less than about 110 nucleotides long, less than about 100 nucleotides long, less than about 90 nucleotides long, less than about 80 nucleotides long, less than about 70 nucleotides long, less than about 60 nucleotides long, less than about 50 nucleotides long, less than about 40 nucleotides long, less than about 30 nucleotides long, or less than about 20 nucleotides long. In one embodiment, the target binding sequence is between 4 and 50 nucleotides in length, such as between 13 and 26 nucleotides long.

In one embodiment, the target binding sequence comprises a sequence reverse complementary to a portion of the Nurr1 mRNA sequence that is at least 18 nucleotides long, such as between 18 and 24 nucleotides long. In another embodiment, the target binding sequence comprises a sequence reverse complementary to a portion of the GDNF mRNA sequence that is at least 14 nucleotides long, such as between 14 and 25 nucleotides long. In another embodiment, the target binding sequence comprises a sequence reverse complementary to a portion of the GBA mRNA sequence that is at least 14 nucleotides long, such as between 14 and 44 nucleotides long. In a further embodiment, the target binding sequence comprises a sequence reverse complementary to a portion of the cRET mRNA sequence that is at least 15 nucleotides long, such as between 15 and 24 nucleotides long.

### Nurr1

The target binding sequence may be designed to hybridise with the 5′ UTR of the Nurr1 mRNA sequence. In one embodiment, the sequence is reverse complementary to 0 to 20 nucleotides, such as 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides of the 5′ UTR. Alternatively, or in combination, the target binding sequence may be designed to hybridise to the CDS of the Nurr1 mRNA sequence. In one embodiment, the sequence is reverse complementary to 0 to 10 nucleotides, such as 0 to 9, 0 to 8, 0 to 7, 0 to 6, 0 to 5, 0 to 4, 0 to 3, 0 to 2, 0 to 1, or 0 nucleotides of the CDS.

The target binding sequence may be designed to hybridise to a region upstream of an AUG site (start codon), such as a start codon within the CDS, of the Nurr1 mRNA sequence. In one embodiment, the sequence is reverse complementary to 0 to 20 nucleotides, such as 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides upstream of the AUG site. Alternatively, or in combination, the target binding sequence may be designed to hybridise to the Nurr1 mRNA sequence downstream of said AUG site. In one embodiment, the sequence is reverse complementary to 0 to 10 nucleotides, such as 0 to 9, 0 to 8, 0 to 7, 0 to 6, 0 to 5, 0 to 4, 0 to 3, 0 to 2, 0 to 1, or 0 nucleotides of the Nurr1 mRNA sequence downstream of said AUG site.

Preferably, the target binding sequence is at least 18 nucleotides long and comprises, from 3′ to 5′:
1) a sequence reverse complementary to 0 to 20 nucleotides of the 5′ UTR and 0 to 4 nucleotides of the CDS of the Nurr1 mRNA sequence; or
2) a sequence reverse complementary to 0 to 18 nucleotides of the region upstream of an AUG site (start codon) of the Nurr1 mRNA and 0 to 4 nucleotides of the CDS of the Nurr1 mRNA sequence downstream of said AUG site.

In case 1) the coding sequence starts on the first AUG site (M1) of the mRNA. In case 2), the preferred AUG site is that corresponding to an internal start codon, such as methionine 44 (M2 at amino acid position M44) or methionine 64 (M3 at amino acid position M64) in exon 3. In the context of referencing a sequence reverse complementary to a region in the 5′ UTR and the CDS, this is preferably anchored around the AUG site, i.e. the region in the 5′ UTR is directly upstream of the AUG site of the target mRNA. For example, reference to a target binding sequence that is “−40/+4 of M1” refers to a target binding sequence that is reverse complementary to the 40 nucleotides within the 5′ UTR upstream of the AUG site (−40) and the 4 nucleotides within the CDS downstream of the AUG site (+4). It will be understood that, under this naming convention, no nucleotide occupies position ‘0’, i.e., position −1 corresponds to the nucleotide immediately upstream of the A of an AUG site and position +1 corresponds to the A of an AUG site.

In one embodiment, the target binding sequence comprises a binding domain as described in Table 1, i.e. with a sequence reverse complementary to the nucleotides of the region upstream and downstream of the methionine in the Nurr1 mRNA sequence (i.e. start codon) as indicated.

In a particular embodiment, the target binding sequence is 18 nucleotides long and comprises, from 3′ to 5′ a sequence reverse complementary to 14 nucleotides of the 5′ UTR and 4 nucleotides of the CDS of the Nurr1 mRNA sequence. For example, the target binding sequence may comprise a sequence encoded by the DNA sequence of SEQ ID NO: 138 (i.e. −14/+4 of M1).

In one particular embodiment, the target binding sequence is 18 nucleotides long and comprises, from 3′ to 5′ a sequence reverse complementary to 14 nucleotides of the region upstream of the AUG site (start codon) and 4 nucleotides of the CDS of the Nurr1 mRNA sequence downstream of said AUG site, for example wherein the AUG site is methionine M2 (corresponding to M44 of the Nurr1 mRNA sequence). For example, the target binding sequence may comprise a sequence encoded by the DNA sequence of SEQ ID NO: 143 (i.e. −14/+4 of M2).

In another particular embodiment, the target binding sequence is 18 nucleotides long and comprises, from 3′ to 5′ a sequence reverse complementary to 18 nucleotides of the region upstream of an AUG site (start codon) and 0 nucleotides of the CDS of the Nurr1 mRNA sequence downstream of said AUG site, for example wherein the AUG site is methionine M2 (corresponding to M44 of the Nurr1 mRNA sequence). For example, the target binding sequence may comprise a sequence encoded by the DNA sequence of SEQ ID NO: 144 (i.e. −18/−1 of M2).

In a further particular embodiment, the target binding sequence is 22 nucleotides long and comprises, from 3′ to 5′ a sequence reverse complementary to 18 nucleotides of the region upstream of an AUG site (start codon) and 4 nucleotides of the CDS of the Nurr1 mRNA sequence downstream of said AUG site, for example wherein the AUG site is methionine M2 (corresponding to M44 of the Nurr1 mRNA sequence). For example, the target binding sequence may comprise a sequence encoded by the DNA sequence of SEQ ID NO: 145 (i.e. −18/+4 of M2).

In a yet further particular embodiment, the target binding sequence is 18 nucleotides long and comprises, from 3′ to 5′ a sequence reverse complementary to 14 nucleotides of the region upstream of an AUG site (start codon) and 4 nucleotides of the CDS of the Nurr1 mRNA sequence downstream of said AUG site, for example wherein the AUG site is methionine M3 (corresponding to M64 of the Nurr1 mRNA sequence). For example, the target binding sequence may comprise a sequence encoded by the DNA sequence of SEQ ID NO: 146 (i.e. −14/+4 of M3).

In another particular embodiment, the target binding sequence is 18 nucleotides long and comprises, from 3′ to 5′ a sequence reverse complementary to 18 nucleotides of the region upstream of an AUG site (start codon) and 0 nucleotides of the CDS of the Nurr1 mRNA sequence downstream of said AUG site, for example wherein the AUG site is methionine M3 (corresponding to M64 of the Nurr1 mRNA sequence). For example, the target binding sequence may comprise a sequence encoded by the DNA sequence of SEQ ID NO: 147 (i.e. −18/−1 of M3).

In one embodiment, there is provided a therapeutic agent for use in a method of treating a disease or disorder of the nervous system wherein the at least one target binding sequence is at least 18 nucleotides long and comprises, from 3′ to 5′:
a sequence reverse complementary to 0 to 20 nucleotides of the 5′ UTR and 0 to 4 nucleotides of the CDS of the Nurr1 mRNA sequence; or
a sequence reverse complementary to 0 to 18 nucleotides of the region upstream of an AUG site (start codon) of the Nurr1 mRNA and 0 to 4 nucleotides of the CDS of the Nurr1 mRNA sequence downstream of said AUG site.

### GDNF

The target binding sequence may be designed to hybridise with the 5′ UTR of the GDNF mRNA sequence. In one embodiment, the sequence is reverse complementary to 0 to 70 nucleotides, such as 0 to 46, 0 to 45, 0 to 44, 0 to 43, 0 to 42, 0 to 41, 0 to 40, 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides of the 5′ UTR. Alternatively, or in combination, the target binding sequence may be designed to hybridise to the CDS of the GDNF mRNA sequence. In one embodiment, the sequence is reverse complementary to 0 to 46 nucleotides, such as 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 or 0 nucleotides of the CDS.

The target binding sequence may be designed to hybridise to a region upstream of an AUG site (start codon), such as a start codon within the CDS, of the GDNF mRNA sequence. In one embodiment, the sequence is reverse complementary to 0 to 25 nucleotides, such as 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides upstream of the AUG site. Alternatively, or in combination, the target binding sequence may be designed to hybridise to the GDNF mRNA sequence downstream of said AUG site. In one embodiment, the sequence is reverse complementary to 0 to 10 nucleotides, such as 0 to 9, 0 to 8, 0 to 7, 0 to 6, 0 to 5, 0 to 4, 0 to 3, 0 to 2, 0 to 1, or 0 nucleotides of the GDNF mRNA sequence downstream of said AUG site.

Preferably, the target binding sequence is at least 14 nucleotides long and comprises, from 3′ to 5′:
1) a sequence reverse complementary to 0 to 56 nucleotides of the 5′ UTR and 0 to 20 nucleotides of the CDS of the GDNF mRNA sequence; or
2) a sequence reverse complementary to 0 to 42 nucleotides of the region upstream of an AUG site (start codon) of the GDNF mRNA and 0 to 4 nucleotides of the CDS of the GDNF mRNA sequence downstream of said AUG site.

In case 1) the coding sequence starts on the first AUG site (M1) of the mRNA. In case 2), the preferred AUG site is that corresponding to an internal start codon, such as methionine 53 (M2 at amino acid position M53) in exon 3, methionine 65 (M3 at amino acid position M65) and/or methionine 83 (M4 at amino acid position M83). In the context of referencing a sequence reverse complementary to a region in the 5′ UTR and the CDS, this is preferably anchored around the AUG site, i.e. the region in the 5′ UTR is directly upstream of the AUG site of the target mRNA. For example, reference to a target binding sequence that is “−40/+4 of M1” refers to a target binding sequence that is reverse complementary to the 40 nucleotides within the 5′ UTR upstream of the AUG site (−40) and the 4 nucleotides within the CDS downstream of the AUG site (+4). It will be understood that, under this naming convention, no nucleotide occupies position ‘0’, i.e., position −1 corresponds to the nucleotide immediately upstream of the A of an AUG site and position +1 corresponds to the A of an AUG site.

In one embodiment, the target binding sequence comprises a binding domain as described in Table 1, i.e. with a sequence reverse complementary to the nucleotides of the region upstream and downstream of the methionine in the GDNF mRNA sequence (i.e. start codon) as indicated.

In a particular embodiment, the target binding sequence is 40 nucleotides long and comprises, from 3′ to 5′ a sequence reverse complementary to 40 nucleotides of the 5′ UTR and 0 nucleotides of the CDS of the GDNF mRNA sequence. For example, the target binding sequence may comprise a sequence encoded by the DNA sequence of SEQ ID NO: 82 (i.e. −40/−1 of M1).

In a particular embodiment, the target binding sequence is 18 nucleotides long and comprises, from 3′ to 5′ a sequence reverse complementary to 14 nucleotides of the 5′ UTR and 4 nucleotides of the CDS of the GDNF mRNA sequence. For example, the target binding sequence may comprise a sequence encoded by the DNA sequence of SEQ ID NO: 85 (i.e. −14/+4 of M1).

In another particular embodiment, the target binding sequence is 21 nucleotides long and comprises, from 3′ to 5′ a sequence reverse complementary to 1 nucleotide of the 5′ UTR and 20 nucleotides of the CDS of the GDNF mRNA sequence. For example, the target binding sequence may comprise a sequence encoded by the DNA sequence of SEQ ID NO: 101 (i.e. −1/+20 of M1).

In a further particular embodiment, the target binding sequence is 18 nucleotides long and comprises, from 3′ to 5′ a sequence reverse complementary to 1 nucleotide of the 5′ UTR and 17 nucleotides of the CDS of the GDNF mRNA sequence. For example, the target binding sequence may comprise a sequence encoded by the DNA sequence of SEQ ID NO: 102 (i.e. −1/+17 of M1).

In one particular embodiment, the target binding sequence is 18 nucleotides long and comprises, from 3′ to 5′ a sequence reverse complementary to 14 nucleotides of the region upstream of an AUG site (start codon) of the GDNF mRNA and 4 nucleotides of the CDS of the GDNF mRNA sequence downstream of said AUG site, for example wherein the AUG site is methionine M2 (corresponding to M53 of the GDNF mRNA sequence). For example, the target binding sequence may comprise a sequence encoded by the DNA sequence of SEQ ID NO: 94 (i.e. −14/+4 of M2).

In one embodiment, there is provided a therapeutic agent for use in a method of treating a disease or disorder of the nervous system, wherein the at least one target binding sequence is at least 14 nucleotides long and comprises, from 3′ to 5′:
a sequence reverse complementary to 0 to 21 nucleotides of the 5′ UTR and 0 to 20 nucleotides of the CDS of the GDNF mRNA sequence; or
a sequence reverse complementary to 0 to 20 nucleotides of the region upstream of an AUG site (start codon) of the GDNF mRNA and 0 to 4 nucleotides of the CDS of the GDNF mRNA sequence downstream of said AUG site.

In one embodiment, the at least one target binding domain is GDNF BDI (−14/−1 M1) (14 nt):

(SEQ ID NO: 205)
CTTAAAGTCCCGTC.

In one embodiment, the at least one target binding domain is GDNF BDII (−14/+4 M1) (18 nt):

(SEQ ID NO: 206)
TCATCTTAAAGTCCCGTC.

In one embodiment, the at least one target binding domain is GDNF BDIII (−15/+4 M1) (19 nt):

(SEQ ID NO: 207)
GCATATTTGAGTCACTGC.

In one embodiment, the at least one target binding domain is GDNF BDIV (−14/+4 M2) (18 nt):

(SEQ ID NO: 208)
GCATATTTGAGTCACTGC.

In one embodiment, the at least one target binding domain is GDNF BDV (−25/−1 M2) (25 nt):

(SEQ ID NO: 209)
ATTTGAGTCACTGCTCAGCGCGAAG.

In one embodiment, the at least one target binding domain is GDNF BDVI (−42/+4 M) (46 nt):

(SEQ ID NO: 210)
CCATGACATCATCGAACTGATCAGGATAATCCTCTGGCATATTTGA.

In one embodiment, the at least one target binding domain has a sequence selected from any one or more of the sequences in the group consisting of SEQ ID NO: 205-210.

cRET

The target binding sequence may be designed to hybridise with the 5′ UTR of the cRET (RET51) mRNA sequence. In one embodiment, the sequence is reverse complementary to 0 to 56 nucleotides, such as 0 to 40, 0 to 38, 0 to 32, 0 to 29, 0 to 24, 0 to 18, 0 to 16, 0 to 15, 0 to 14, 0 to 13, 0 to 11, 0 to 5, 0 to 4, or 0 to 1 nucleotides of the 5′ UTR. Alternatively, or in combination, the target binding sequence may be designed to hybridise to the CDS of the CRET (RET51) mRNA sequence. In one embodiment, the sequence is reverse complementary to 0 to 50 nucleotides, such as 0 to 49, 0 to 48, 0 to 47, 0 to 46, 0 to 45, 0 to 44, 0 to 43, 0 to 42, 0 to 41, 0 to 40, 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides of the CDS.

The target binding sequence may be designed to hybridise to a region upstream of an AUG site (start codon), such as a start codon within the CDS, of the cRET mRNA sequence. In one embodiment, the sequence is reverse complementary to 0 to 20 nucleotides, such as 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides upstream of the AUG site. Alternatively, or in combination, the target binding sequence may be designed to hybridise to the cRET mRNA sequence downstream of said AUG site. In one embodiment, the sequence is reverse complementary to 0 to 15 nucleotides, such as 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides of the cRET mRNA sequence downstream of said AUG site.

Preferably, the target binding sequence is at least 14 nucleotides long and comprises, from 3′ to 5′:
1) a sequence reverse complementary to 0 to 56 nucleotides of the 5′ UTR and 0 to 23 nucleotides of the CDS of the cRET mRNA sequence; or
2) a sequence reverse complementary to 0 to 50 nucleotides of the region upstream of an AUG site (start codon) of the cRET mRNA and 0 to 19 nucleotides of the CDS of the CRET mRNA sequence downstream of said AUG site.

In case 1) the coding sequence starts on the first AUG site (M1) of the mRNA. In case 2), the preferred AUG site is that corresponding to an internal start codon, such as methionine 255 (M2 at amino acid position M255) and/or methionine 370 (M3 at amino acid position M370). In the context of referencing a sequence reverse complementary to a region in the 5′ UTR and the CDS, this is preferably anchored around the AUG site, i.e. the region in the 5′ UTR is directly upstream of the AUG site of the target mRNA. For example, reference to a target binding sequence that is “−40/+4 of M1” refers to a target binding sequence that is reverse complementary to the 40 nucleotides within the 5′ UTR upstream of the AUG site (−40) and the 4 nucleotides within the CDS downstream of the AUG site (+4). It will be understood that, under this naming convention, no nucleotide occupies position ‘0’, i.e., position −1 corresponds to the nucleotide immediately upstream of the A of an AUG site and position +1 corresponds to the A of an AUG site.

In one embodiment, the target binding sequence comprises a binding domain as described in Table 1, i.e. with a sequence reverse complementary to the nucleotides of the region upstream and downstream of the methionine in the cRET mRNA sequence (i.e. start codon) as indicated.

In a particular embodiment, the target binding sequence is 40 nucleotides long and comprises, from 3′ to 5′ a sequence reverse complementary to 40 nucleotides of the 5′ UTR and 0 nucleotides of the CDS of the cRET mRNA sequence. For example, the target binding sequence may comprise a sequence encoded by the DNA sequence of SEQ ID NO: 110 (i.e. −40/−1 of M1).

In a particular embodiment, the target binding sequence is 18 nucleotides long and comprises, from 3′ to 5′ a sequence reverse complementary to 14 nucleotides of the 5′ UTR and 4 nucleotides of the CDS of the cRET mRNA sequence. For example, the target binding sequence may comprise a sequence encoded by the DNA sequence of SEQ ID NO: 112 (i.e. −14/+4 of M1).

In one particular embodiment, the target binding sequence is 15 nucleotides long and comprises, from 3′ to 5′ a sequence reverse complementary to 11 nucleotides of the 5′ UTR and 4 nucleotides of the CDS of the cRET mRNA sequence. For example, the target binding sequence may comprise a sequence encoded by the DNA sequence of SEQ ID NO: 129 (i.e. −11/+4 of M1).

In another particular embodiment, the target binding sequence is 18 nucleotides long and comprises, from 3′ to 5′ a sequence reverse complementary to 11 nucleotides of the 5′ UTR and 7 nucleotides of the CDS of the cRET mRNA sequence. For example, the target binding sequence may comprise a sequence encoded by the DNA sequence of SEQ ID NO: 130 (i.e. −11/+7 of M1).

In one particular embodiment, the target binding sequence is 18 nucleotides long and comprises, from 3′ to 5′ a sequence reverse complementary to 14 nucleotides of the region upstream of an AUG site (start codon) of the cRET mRNA and 4 nucleotides of the CDS of the cRET mRNA sequence downstream of said AUG site, for example wherein the AUG site is methionine M2 (corresponding to M255 of the cRET mRNA sequence). For example, the target binding sequence may comprise a sequence encoded by the DNA sequence of SEQ ID NO: 118 (i.e. −14/+4 of M2).

In another embodiment, the target binding sequence is 40 nucleotides long and comprises, from 3′ to 5′ a sequence reverse complementary to 40 nucleotides of the region upstream of an AUG site (start codon) of the cRET mRNA and 0 nucleotides of the CDS of the cRET mRNA sequence downstream of said AUG site, for example wherein the AUG site is methionine M2 (corresponding to M255 of the cRET mRNA sequence). For example, the target binding sequence may comprise a sequence encoded by the DNA sequence of SEQ ID NO: 119 (i.e. −40/−1 of M2).

In one embodiment, there is provided a therapeutic agent for use in a method of treating a disease or disorder of the nervous system, wherein the at least one target binding sequence is at least 15 nucleotides long and comprises, from 3′ to 5″:
a sequence reverse complementary to 0 to 14 nucleotides of the 5′ UTR and 0 to 23 nucleotides of the CDS of the cRET mRNA sequence; or
a sequence reverse complementary to 0 to 14 nucleotides of the region upstream of an AUG site (start codon) of the cRET mRNA and 0 to 13 nucleotides of the CDS of the cRET mRNA sequence downstream of said AUG site.

### GBA

The target binding sequence may be designed to hybridise with the 5′-untranslated region (5′ UTR) of the GBA mRNA sequence. In one embodiment, the sequence is reverse complementary to 0 to 56 nucleotides, such as 0 to 55, 0 to 54, 0 to 53, 0 to 52, 0 to 51, 0 to 50, 0 to 49, 0 to 48, 0 to 47, 0 to 46, 0 to 45, 0 to 44, 0 to 43, 0 to 42, 0 to 41, 0 to 40, 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides of the 5′ UTR. Alternatively, or in combination, the target binding sequence may be designed to hybridise to the coding sequence (CDS) of the GBA mRNA sequence. In one embodiment, the sequence is reverse complementary to 0 to 20 nucleotides, such as 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides of the CDS.

The target binding sequence may be designed to hybridise to a region upstream of an AUG site (start codon), such as a start codon within the CDS, of the GBA mRNA sequence. In one embodiment, the sequence is reverse complementary to 0 to 20 nucleotides, such as 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides upstream of the AUG site. Alternatively, or in combination, the target binding sequence may be designed to hybridise to the GBA mRNA sequence downstream of said AUG site. In one embodiment, the sequence is reverse complementary to 0 to 10 nucleotides, such as 0 to 9, 0 to 8, 0 to 7, 0 to 6, 0 to 5, 0 to 4, 0 to 3, 0 to 2, or 0 to 1 nucleotides of the GBA mRNA sequence downstream of said AUG site.

Preferably, the target binding sequence is at least 14 nucleotides long and comprises, from 3′ to 5′:
1) a sequence reverse complementary to 0 to 56 nucleotides of the 5′ UTR and 0 to 14 nucleotides of the CDS of the GBA mRNA sequence; or
2) a sequence reverse complementary to 0 to 40 nucleotides of the region upstream of an AUG site (start codon) of the GBA mRNA and 0 to 12 nucleotides of the CDS of the GBA mRNA sequence downstream of said AUG site.

In case 1) the coding sequence starts on the first AUG site (M1) of the mRNA. In case 2), the preferred AUG site is that corresponding to an internal start codon. In the context of referencing a sequence reverse complementary to a region in the 5′ UTR and the CDS, this is preferably anchored around the AUG site, i.e. the region in the 5′ UTR is directly upstream of the AUG site of the target mRNA. For example, reference to a target binding sequence that is “−40/+4 of M1” refers to a target binding sequence that is reverse complementary to the 40 nucleotides within the 5′ UTR upstream of the AUG site (−40) and the 4 nucleotides within the CDS downstream of the AUG site (+4). It will be understood that, under this naming convention, no nucleotide occupies position ‘0’, i.e., position −1 corresponds to the nucleotide immediately upstream of the A of an AUG site and position +1 corresponds to the A of an AUG site.

In one embodiment, the target binding sequence comprises a binding domain as described in Table 1, i.e. with a sequence reverse complementary to the nucleotides of the region upstream and downstream of the methionine in the GBA mRNA sequence (i.e. start codon) as indicated.

Preferably, the target binding sequence is between 14 and 44 nucleotides long and comprises, from 3′ to 5′, a sequence reverse complementary to up to 40 nucleotides of the region upstream of a start codon of the GBA mRNA sequence and up to 4 nucleotides of the GBA mRNA sequence downstream of said start codon. The region upstream of the start codon may be within the 5′ UTR (if the start codon is the first AUG site, i.e. M1) or within the CDS (if the start codon is an internal start codon, such as M2 at amino acid position M21, M3 at amino acid position M88, M6 at amino acid position M162 or M9 at amino acid position M400).

In a particular embodiment, the target binding sequence is 44 nucleotides long and comprises, from 3′ to 5′ a sequence reverse complementary to 40 nucleotides of the 5′ UTR and 4 nucleotides of the CDS of the GBA mRNA sequence. For example, the target binding sequence may comprise a sequence encoded by the DNA sequence of SEQ ID NO: 152 (i.e. −40/+4 of M1).

In another embodiment, the target binding sequence is 14 nucleotides long and comprises, from 3′ to 5′ a sequence reverse complementary to 14 nucleotides of the 5′ UTR and 0 nucleotides of the CDS of the GBA mRNA sequence. For example, the target binding sequence may comprise a sequence encoded by the DNA sequence of SEQ ID NO: 153 (i.e. −14/−1 of M1).

In another embodiment, the target binding sequence is 40 nucleotides long and comprises, from 3′ to 5′ a sequence reverse complementary to 40 nucleotides of the region upstream of an AUG site (start codon) of the GBA mRNA and 0 nucleotides of the CDS of the GBA mRNA sequence downstream of said AUG site, wherein the AUG site is methionine M2 (corresponding to M21 of the GBA mRNA sequence). For example, the target binding sequence may comprise a sequence encoded by the DNA sequence of SEQ ID NO: 159 (i.e. −40/−1 of M2).

In one embodiment, there is provided a therapeutic agent for use in a method of treating a disease or disorder of the nervous system, wherein the at least one target binding sequence is at least 15 nucleotides long and comprises, from 3′ to 5′:
a sequence reverse complementary to 0 to 56 nucleotides of the 5′ UTR and 0 to 14 nucleotides of the CDS of the GBA mRNA sequence; or
a sequence reverse complementary to 0 to 40 nucleotides of the region upstream of an AUG site (start codon) of the GBA mRNA and 0 to 12 nucleotides of the CDS of the GBA mRNA sequence downstream of said AUG site.

In one embodiment, the target binding sequence comprises a sequence with at least 75% sequence identity, at least 80% sequence identity, at least 85% sequence identity, preferably at least 90% sequence identity, at least 91% sequence identity, at least 92% sequence identity, at least 93% sequence identity, at least 94% sequence identity, more preferably at least 95% sequence identity, at least 96% sequence identity, at least 97% sequence identity, at least 98% sequence identity, at least 99% sequence identity, even more preferably 100% sequence identity to any of SEQ ID NOs: 70-157, preferably SEQ ID NOs: 70, 75-79, 82, 85, 94, 101, 102, 110, 112, 118, 119, 129, 130, 142, 143 or 149. In a further embodiment, the target binding sequence consists of a sequence with at least 75% sequence identity, at least 80% sequence identity, at least 85% sequence identity, preferably at least 90% sequence identity, at least 91% sequence identity, at least 92% sequence identity, at least 93% sequence identity, at least 94% sequence identity, more preferably at least 95% sequence identity, at least 96% sequence identity, at least 97% sequence identity, at least 98% sequence identity, at least 99% sequence identity, even more preferably 100% sequence identity to any of SEQ ID NOs: 70-157, preferably SEQ ID NOs: 70, 75-79, 82, 85, 94, 101, 102, 110, 112, 118, 119, 129, 130, 142, 143 or 149.

More specifically, SEQ ID NOs: 70-79 relate to target binding sequences directed to human Nurr1 isoforms, SEQ ID NOs: 80-105 relate to target binding sequences directed to human GDNF isoforms, SEQ ID NOs: 106-137 relate to target binding sequences directed to human CRET isoforms, SEQ ID NOs: 138-157 relate to target binding sequences directed to human GBA isoforms. However, as will be appreciated from the sequence identity of human GBA, GDNF, cRET and Nurr1 isoforms to those of, for example, mouse and rhesus macaque (Macaca mulatta), the target binding sequences presented herein may have cross-reactivity with other species.

For example, human Nurr1 mRNA has 98.70% sequence identity across its entire length with Macaca mulatta Nurr1 mRNA and 91.82% sequence identity with mouse Nurr1 mRNA. Thus, in a yet further embodiment, the target binding sequence comprising or consisting of any of SEQ ID NOs: 70-79 binds Macaca mulatta Nurr1 and/or mouse Nurr1. In another example, human GDNF mRNA has 95.61% sequence identity across its entire length with Macaca mulatta GDNF mRNA. Thus, in another embodiment, the target binding sequence comprising or consisting of any of SEQ ID NOs: 80-105 binds Macaca mulatta GDNF. In a further example, human cRET mRNA has 96.41% sequence identity across its entire length with Macaca mulatta cRET mRNA and 79.20% sequence identity with mouse cRET mRNA. Thus, in a further embodiment, the target binding sequence comprising or consisting of any of SEQ ID NOs: 106-137 binds Macaca mulatta cRET and/or mouse cRET. In a yet further example, human GBA mRNA has 96.73% sequence identity across its entire length with Macaca mulatta GBA mRNA and 80.42% sequence identity with mouse GBA mRNA. Thus, in a yet further embodiment, the target binding sequence comprising or consisting of any of SEQ ID NOs: 138-157 binds Macaca mulatta GBA and/or mouse GBA.

The target binding sequences utilised in the examples herein are named according to their target and are each given a numerical identifier. For example hGBA BD01 (or BD1 or BDI) will be understood as a binding domain to hGB1. Thus a ‘BD1’ quoted in relation to a given target mRNA will not be the same as a ‘BD1’ for a different target mRNA.

In one embodiment, the functional nucleic acid molecules provided herein are chemically modified. The term “modification” or “chemical modification” refers to a structural change in, or on, the most common, natural ribonucleotides: adenosine, guanosine, cytidine, or uridine ribonucleotides. Chemical modifications may be changes in or on a nucleobase (i.e. a chemical base modification), or in or on a sugar (i.e. a chemical sugar modification). The chemical modifications may be introduced co-transcriptionally (e.g. by substitution of one or more nucleotides with a modified nucleotide during synthesis), or post-transcriptionally (e.g. by the action of an enzyme).

Chemical modifications are known in the art, for example as described in The RNA Modification Database provided by The RNA Institute (https://mods.rna.albany.edu/mods/).

Many modifications occur in nature, such as chemical modifications to natural transfer RNAs (tRNAs), which include, for example: 2′-O-Methyl (such as 2′-O-Methyladenosine, 2′-O-Methylguanosine and 2′-O-Methylpseudouridine), 1-Methyladenosine, 2-Methyladenosine, 1-Methylguanosine, 7-Methylguanosine, 2-Thiocytidine, 5-Methylcytidine, 5-Formylcytidine, Pseudouridine, Dihydrouridine, or the like.

Examples of chemical modifications which may be useful in the present invention are described in PCT/GB2021/052607, which is incorporated herein by reference in its entirety.

### Structural Features

The functional nucleic acid molecule may comprise more than one regulatory sequence, which can be the same sequence repeated more than once, or a different regulatory sequence (i.e. a different SINE B2 element/functionally active fragment of a SINE B2 element/an IRES sequence/an IRES derived sequence/functionally active fragment of an IRES sequence).

The at least one target binding sequence and the at least one regulatory sequence are preferably connected by at least one spacer/linker sequence. SEQ ID NOs: 203 or 204 are non-limiting examples of the spacer/linker sequence that may be used in the present invention.

In one embodiment, the functional nucleic acid molecule comprises at least one linker sequence between the at least one target binding sequence and the at least one regulatory sequence.

The functional nucleic acid molecule of the present invention is preferably a circular molecule. This conformation leads to a much more stable molecule that is degraded with greater difficulty within the cell (exonucleases cannot degrade circular molecules) and therefore remains active for a longer time.

Furthermore, the functional nucleic acid molecule may optionally comprise a non-coding 3′ tail sequence, which e.g. includes restriction sites useful for cloning the molecule in appropriate plasmids.

In one embodiment, the functional nucleic acid molecule of the invention is circular.

In one embodiment, the functional nucleic acid molecule comprises a 3′-polyadenylation (polyA) tail. A “3′-polyA tail” refers to a long chain of adenine nucleotides added to the 3′-end of the transcription which provides stability to the RNA molecule and can promote translation.

In one embodiment the functional nucleic acid molecule comprises a 5′-cap. A “5′-cap” refers to an altered nucleotide at the 5′-end of the transcript which provides stability to the molecule, particularly from degradation from exonucleases, and can promote translation. Most commonly, the 5′-cap may be a 7-methylguanylate cap (m7G), i.e. a guanine nucleotide connected to the RNA via a 5′ to 5′ triphosphate linkage and methylated on the 7 position.

It should be noted that the functional nucleic acid molecules can enhance translation of the target gene of interest with no effects on mRNA quantities of the target gene. Therefore, they can successfully be used as molecular tools to validate gene function in cells as well as to implement the pipelines of recombinant protein production.

### DNA Molecules and Vectors

According to a further aspect of the invention, there is provided a DNA molecule encoding any of the functional nucleic acid molecules disclosed herein. According to a further aspect of the invention, there is provided an expression vector comprising said DNA molecule.

Exemplary expression vectors are known in the art and may include, for example, plasmid vectors, viral vectors (for example adenovirus, adeno-associated virus, retrovirus or lentivirus vectors), phage vectors, cosmid vectors and the like. The choice of expression vector may be dependent upon the type of host cell to be used and the purpose of use. In particular the following plasmids have been used for efficient expression of functional nucleic acid molecules:

### Mammalian Expression Plasmids:

Plasmid Name: pCDNA3.1 (−)
Expression: CMV promoter
   BGH poly(A) terminator
Plasmid Name: pDUAL-eGFPA (modified from peGFP-C2)
Expression: H1 promoter
   BGH poly(A) terminator
Plasmid Name: pCS2+ link (modified from pCS2+)
Expression: CMV IE94 promoter
   SV40 poly(A) signal

### Viral Vectors:

Vector Name: pAAV
Virus: Adeno-Associated Virus
Expression: CAG promoter/CMV enhancer
   SV40 late poly(A) terminator
Vector Name: rcLV-TetOne-Puro
Virus: Lentivirus (3rd generation)
Expression: LTR-TREt (Tre-Tight) promoter (doxycycline-inducible expression)
   BGH poly(A) terminator
Vector Name: pLPCX-link
Virus: Retrovirus (3rd generation)
Expression: CMV

It should be noted that any promoter may be used in the vector and will work just as well as those mentioned above.

### Compositions

The present invention also relates to compositions comprising the functional nucleic acid molecules, the DNA molecules, or the expression vectors described herein. The composition and pharmaceutical composition may comprise components which enable delivery of said functional nucleic acid molecules by viral vectors (AAV, lentivirus and the like) and non-viral vectors (nanoparticles, lipid particles and the like).

The functional nucleic acid molecule of the invention may also be administered as naked or unpackaged RNA. In one embodiment, the functional nucleic acid molecule is administered as naked RNA.

Alternatively, the functional nucleic acid molecules, the DNA molecules, or the expression vectors may be administered as part of a composition, for example compositions comprising a suitable carrier. In certain embodiments, the carrier is selected based upon its ability to facilitate the transfection of a target cell with one or more functional nucleic acid molecules, DNA molecules, or expression vectors according to the present invention.

In one aspect, there is provided a composition comprising at least one functional nucleic acid molecule, at least one DNA molecule, or at least one expression vector according to the present invention.

In one aspect, there is provided a pharmaceutical composition comprising at least one functional nucleic acid molecule, at least one DNA molecule, or at least one expression vector according to the present invention.

Suitably, a pharmaceutical composition may comprise at least one functional nucleic acid molecule, at least one DNA molecule, or at least one expression vector according to the present invention in admixture with a suitable pharmaceutical excipient, diluent or carrier with regard to the intended route of administration and standard pharmaceutical practice.

According to a further aspect of the invention, there is provided the functional nucleic acid molecule, DNA molecule, expression vector, composition, or pharmaceutical composition as defined herein for use as a medicament.

It will be understood that the functional nucleic acid molecules of the invention find use in increasing the level of Nurr1, GDNF, cRET or GBA protein within a cell. Nurr1, GDNF, cRET and GBA have functions in the dopaminergic system of the brain, therefore according to a further aspect of the invention there is provided the functional nucleic acid molecule, the DNA molecule, the composition, or the pharmaceutical composition as defined herein, for use in a method of treating a disease or disorder of the nervous system, such as the central nervous system.

According to one aspect of the invention, there is provided a functional nucleic acid molecule for use in a method of treating a disease or disorder of the nervous system, wherein the functional nucleic acid molecule increases translation of an endogenous mRNA sequence selected from the group consisting of: a Nurr1, GDNF, cRET or GBA mRNA sequence.

The above said functional nucleic acid molecules, DNA molecules, expression vectors, compositions and/or pharmaceutical compositions are used as medicaments, preferably for treating a neurodegenerative disease or disorder, such as Parkinson's disease.

Therefore, in a further embodiment, the disease or disorder of the nervous system is a neurodegenerative disorder, such as Parkinson's disease. Parkinson's disease is one of the most common neurodegenerative disorders and is caused by the loss of dopamine neurons of the substantia nigra pars compacta (SNpc). The main symptoms are well known and include motor-related defects, such as rigidity and tremor. Although Parkinson's disease may be familial (about 5% of cases), most patients are sporadic.

According to a further aspect of the invention, there is provided the use of the functional nucleic acid molecule (or DNA molecule, expression vector, composition, or pharmaceutical composition) as defined herein for the manufacture of a medicament for the treatment of a disease or disorder of the nervous system, such as a neurodegenerative disorder, for example Parkinson's disease.

In one embodiment the disease or disorder to be treated is Parkinson's disease.

### Therapeutic Uses and Methods

According to a further aspect of the invention, there is provided a method for enhancing protein translation of Nurr1, GDNF, cRET or GBA mRNA in a cell comprising administering the functional nucleic acid molecule, DNA molecule, expression vector, composition or pharmaceutical composition as defined herein to the cell. Preferably, the cell is a mammalian cell, such as a human, mouse or rhesus monkey cell. Most preferably, the cell is a human cell.

According to a further aspect of the invention, there is provided a method for increasing the protein synthesis efficiency of target protein selected from the group consisting of: a Nurr1, GDNF, cRET or GBA protein, in a cell comprising administering the functional nucleic acid molecule, DNA molecule, expression vector or composition, or pharmaceutical composition as defined herein, to the cell.

Thus, in some embodiments, the translation of endogenous human Nurr1, GDNF, cRET or GBA mRNA is increased. In other embodiments, the translation of mouse Nurr1, cRET or GBA mRNA is increased.

The methods described herein may comprise transfecting into a cell the functional nucleic acid molecule, DNA molecule, expression vector, composition or pharmaceutical composition as defined herein. The functional nucleic acid molecule, DNA molecule, expression vector, composition or pharmaceutical composition may be administered to target cells using methods known in the art and include, for example, microinjection, lipofection, electroporation, using calcium phosphate, self-infection by the vector or transduction of a virus. Suitable methods for gene delivery are known in the art, for example those described in Sung & Kim (2019) Biomater. Res. 23: 8.

The functional nucleic acid molecules as described herein (e.g. a SINEUP) might be used to directly compensate a precise deficiency in the expression of the protein it targets. The molecules can be designed to increase a target protein level in a disease where the target protein is mutated and the resulting low level of target protein causes the pathology. Such a deficiency could be the result of a genetic mutation leading to haploinsufficiency. In that model, the functional nucleic acid molecule (e.g. a SINEUP) directly restores the target protein level where the level is a cause of the disease.

However, some targets (e.g. Nurr1, GDNF and cRET as described herein) are also important “controllers” of beneficial pathways in the CNS, such as pathways for neuroprotection, neurorestoration, neuroregeneration, or for controlling neuroinflammation. They are neurotrophic factors, growth factors, receptors or transcription factors that are key in transmission of neuroprotective signals. The functional nucleic acid molecules as described herein which boost these proteins could be therapeutically beneficial even though the lack of expression of the proteins themselves might not be the actual root cause of the disease. Such SINEUPs could be used to boost the expression of factors chosen for their functional role in supporting healthy cell function, cell survival or cell repair mechanism, independently of whether that particular factor is specifically mutated or depleted in the indication.

Such molecules may therefore be referred to as “beneficial” functional nucleic acid molecules, e.g., SINEUPs, and would have a wider indication than the those previously designed to specifically repair a protein deficiency caused by a mutation. For example, therapies aimed to replenish the level of neurotrophic factors such as GDNF may provide important neuronal support in a variety of neurodegenerative pathologies including Alzheimer's (AD), Parkinson's (PD), Huntington's (HD), amyotrophic lateral sclerosis (ALS) or Rett Syndrome (Allen et al. (2013) Pharmacol. Ther. 138 (2): 155-175).

In one embodiment, the cell is Nurr1, GDNF, cRET or GBA haploinsufficient, i.e. wherein the presence of a variant allele in a heterozygous combination results in the amount of product generated by the single wild-type gene is not sufficient for complete or normal function. Generally, haploinsufficiency is a condition that arises when the normal phenotype requires the protein product of both alleles, and reduction to 50% or less of gene function results in an abnormal phenotype.

Methods of the invention result in increased levels of Nurr1, GDNF, cRET or GBA protein in a cell and therefore find use, for example, in methods of treatment for diseases which are associated with Nurr1, GDNF, cRET or GBA defects (i.e. reduced protein levels and/or loss-of-function mutations of these genes). Methods of the invention find particular use in diseases caused by a quantitative decrease in the predetermined, normal protein level. Methods of the invention can be performed in vitro, ex vivo or in vivo.

Thus, according to one particular aspect of the invention, there is provided a functional nucleic acid molecule for use in a method of treating a disease or disorder of the nervous system (such as the central nervous system, in particular a neurodegenerative disorder) wherein the functional nucleic acid molecule increases translation of endogenous mRNA sequence selected from the group consisting of: a Nurr1, GDNF, cRET or GBA mRNA sequence.

According to a further aspect of the invention, there is provided a method of treating a disease or disorder of the nervous system comprising administering a therapeutic agent that increases translation of an endogenous mRNA sequence selected from the group consisting of: an endogenous Nurr1, GDNF, cRET or GBA mRNA sequence to a subject with a disease or disorder of the nervous system.

According to a further aspect of the invention, there is provided a method of treating a disease or disorder of the nervous system (such as a neurodegenerative disorder, for example Parkinson's disease), comprising administering a therapeutically effective amount of the functional nucleic acid molecule, DNA molecule, expression vector, composition, or pharmaceutical composition as defined herein, to a subject with a disease or disorder of the nervous system.

Gene therapy in neurological diseases, such as neurodegenerative disorders including Parkinson's disease, is challenging because of the requirement to cross the blood-brain barrier (BBB). The BBB is a highly selective semipermeable barrier which prevents solutes in the circulating blood from non-selectively crossing into the extracellular fluid of the central nervous system. Thus, in one embodiment, the functional nucleic acid molecule, DNA molecule or expression vector as defined herein is comprised in a blood-brain barrier-crossing vector, such as a neurotropic viral vector. In a further embodiment, the composition or pharmaceutical composition as defined herein comprises a neurotropic viral vector. In certain embodiments, the neurotropic viral vector is an adeno-associated viral vector, such as AAV9.

In one embodiment the therapeutically effective amount is administered in the nervous system, such as the central nervous system, in particular to the brain.

Homozygous mutations in the GBA gene are also linked to Gaucher's disease (GD), a multi-organ disease that is generally classed as a metabolic disorder, in particular a lysosomal storage disorder. GD can be divided into at least four types, some of which are associated with neuropathology (Gaucher disease type 2, also known as acute neuronopathic Gaucher disease, and Gaucher disease type 3, also known as chronic neuronopathic Gaucher disease). Thus, according to one particular aspect of the invention, there is provided a functional nucleic acid molecule for use in a method of treating Gaucher's disease wherein the functional nucleic acid molecule increases translation of endogenous GBA mRNA sequence.

According to a further aspect of the invention, there is provided a method of treating Gaucher's disease comprising administering a therapeutic agent that increases translation of an endogenous GBA mRNA sequence to a subject with Gaucher's disease.

According to a further aspect of the invention, there is provided a method of treating Gaucher's disease, comprising administering a therapeutically effective amount of the functional nucleic acid molecule, DNA molecule, expression vector, composition or pharmaceutical composition as defined herein, wherein the target mRNA is a GBA mRNA sequence to a subject with Gaucher's disease.

### Model Systems

Genetic analysis of familiar Parkinson's Disease (PD) has established that the presence of SNCA gene duplication is sufficient to cause PD in humans. In idiopathic PD, subtle rather than large cellular increased α-synuclein expression is found in compromised neurons. These observations suggest that an increase of two-fold of α-synuclein expression throughout the lifetime of an individual is able to provoke both motor and non-motor symptoms of the disease. Current technologies used to establish non-human animal models of PD take advantage of neurochemical intoxications or of ectopic expression of mutant α-synuclein by viral delivery of its cDNA into the brain. In the latter case, the level of expression is usually much higher and less controlled than the one occurring in patients. Described herein is a model of PD which takes advantage of viral delivery of SINEUP-SNCA by using a long non-coding RNA antisense to the endogenous SNCA transcript (microSINEUP-SNCA). SINEUPs are able to increase translation and therefore protein levels of the target gene in the two-fold range, therefore mimicking the expression dynamics naturally occurring in PD patients. Functional nucleic acids that comprise IRES sequences or functionally active fragments of IRES sequences, according to the present invention, may act analogously to SINE B2 and SINE B2 fragment containing functional nucleic acids, e.g., SINEUPs. This approach will allow studying the effects of increased α-synuclein expression in neuronal cells, thereby testing the differential contribution of dopaminergic subpopulations on motor and affective (anxiety/depression) symptoms of PD, respectively.

Therefore, according to one aspect of the invention, there is provided a method of producing a non-human animal model of PD, particularly a non-human primate (NHP) model of PD, comprising: administering a functional nucleic acid molecule comprising at least one target binding sequence comprising a sequence reverse complementary to a SNCA mRNA sequence; and at least one regulatory sequence comprising a SINE B2 element, a functionally active fragment of a SINE B2 element, an IRES sequence or a functionally active fragment of an IRES sequence, to a non-human animal.

According to another aspect of the invention, there is provided a method of producing a non-human animal model of PD, particularly a non-human primate (NHP) model of PD, comprising: administering the functional nucleic acid molecule, the DNA molecule, the expression vector, the composition, or the pharmaceutical composition as defined herein, to a non-human animal; wherein, the functional nucleic acid molecule comprises:
at least one target binding sequence comprising a sequence reverse complementary to a SNCA mRNA sequence; and
at least one regulatory sequence comprising a SINE B2 element, a functionally active fragment of a SINE B2 element, an IRES sequence or a functionally active fragment of an IRES sequence.

In one embodiment, the non-human animal is a non-human primate.

Following administration of the functional nucleic acid molecule, the DNA molecule, the expression vector, the composition, or the pharmaceutical composition, the non-human animal will exhibit symptoms of Parkinson's disease. The functional nucleic acid molecule enhances protein translation of SNCA mRNA thereby reproducing the cellular impact of α-synuclein accumulation which is associated with comorbid anxiety/depression-like behaviour and motor deficits. Symptoms of PD would include, but are not limited to, progressive development of classic PD pathology (i.e. loss of neurons), loss of Dorsal Raphe Nucleus Dopamine (DRNDA) neurons and associated anxiety/depression-like behaviour.

Embodiments described herein in relation to functional nucleic acid molecules, DNA molecules, expression vectors compositions or pharmaceutical compositions may be applied to the SNCA mRNA sequence. For example, the functional nucleic acid molecule may be administered in any pharmaceutically acceptable carrier and any route of administration as discussed herein. It will be understood that effective amounts of the functional nucleic acid molecule will vary with the route of administration, and the severity of PD disease state desired.

According to a further aspect of the invention, there is provided a method for enhancing protein translation of SNCA mRNA in a cell comprising administering the functional nucleic acid molecule, DNA molecule, expression vector, composition or pharmaceutical composition as defined herein to the cell. Preferably the cell is a non-human, mammalian cell, such as a non-human primate or mouse cell.

According to a further aspect of the invention, there is provided a method for increasing the protein synthesis efficiency of SNCA in a cell comprising administering the functional nucleic acid molecule, DNA molecule, expression vector, composition or pharmaceutical composition as defined herein, to the cell.

In some embodiments, the translation of endogenous non-human primate SNCA mRNA is increased. In other embodiments, the translation of mouse SNCA mRNA is increased.

In one embodiment, the target binding sequence comprises a sequence reverse complementary to a portion of the SNCA mRNA sequence that is at least 14 nucleotides long, such as between 14 and 70 nucleotides long.

The target binding sequence may be designed to hybridise with the 5′-untranslated region (5′ UTR) of the SNCA mRNA sequence. In one embodiment, the sequence is reverse complementary to 0 to 54 nucleotides, such as 0 to 50, 0 to 49, 0 to 48, 0 to 47, 0 to 46, 0 to 45, 0 to 44, 0 to 43, 0 to 42, 0 to 41, 0 to 40, 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides of the 5′ UTR. Alternatively, or in combination, the target binding sequence may be designed to hybridise to the coding sequence (CDS) of the SNCA mRNA sequence. In one embodiment, the sequence is reverse complementary to 0 to 46 nucleotides, such as 0 to 44, 0 to 43, 0 to 42, 0 to 41, 0 to 40, 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides of the CDS.

The target binding sequence may be designed to hybridise to a region upstream of an AUG site (start codon), such as a start codon within the CDS, of the SNCA mRNA sequence. In one embodiment, the sequence is reverse complementary to 0 to 20 nucleotides, such as 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, or 0 to 1 nucleotides upstream of the AUG site. Alternatively, or in combination, the target binding sequence may be designed to hybridise to the SNCA mRNA sequence downstream of said AUG site. In one embodiment, the sequence is reverse complementary to 0 to 10 nucleotides, such as 0 to 9, 0 to 8, 0 to 7, 0 to 6, 0 to 5, 0 to 4, 0 to 3, 0 to 2, or 0 to 1 nucleotides of the SNCA mRNA sequence downstream of said AUG site.

Preferably, the target binding sequence is at least 14 nucleotides long and comprises, from 3′ to 5′:
1) a sequence reverse complementary to 0 to 54 nucleotides of the 5′ UTR and 0 to 16 nucleotides of the CDS of the SNCA mRNA sequence; or
2) a sequence reverse complementary to 0 to 40 nucleotides of the region upstream of an AUG site (start codon) of the SNCA mRNA and 0 to 37 nucleotides of the CDS of the SNCA mRNA sequence downstream of said AUG site.

In case 1) the coding sequence starts on the first AUG site (M1) of the mRNA. In case 2), the preferred AUG site is that corresponding to an internal start codon. In the context of referencing a sequence reverse complementary to a region in the 5′ UTR and the CDS, this is preferably anchored around the AUG site, i.e. the region in the 5′ UTR is directly upstream of the AUG site of the target mRNA. For example, reference to a target binding sequence that is “−40/+4 of M1” refers to a target binding sequence that is reverse complementary to the 40 nucleotides within the 5′ UTR upstream of the AUG site (−40) and the 4 nucleotides within the CDS downstream of the AUG site (+4). It will be understood that, under this naming convention, no nucleotide occupies position ‘0’, i.e., position −1 corresponds to the nucleotide immediately upstream of the A of an AUG site and position +1 corresponds to the A of an AUG site.

Preferably, the target binding sequence is 18 nucleotides long and comprises, from 3′ to 5′, a sequence reverse complementary to 14 nucleotides of the region upstream of a start codon of the SNCA mRNA sequence and 4 nucleotides of the SNCA mRNA sequence downstream of said start codon. The region upstream of the start codon may be within the 5′ UTR (if the start codon is the first AUG site, i.e. M1) or within the CDS (if the start codon is an internal start codon, such as M2 at amino acid position M5, M3 at amino acid position M100 and/or M5 at amino acid position M127).

In one embodiment, the target binding sequence comprises a binding domain as described in Table 7, i.e. with a sequence reverse complementary to the nucleotides of the region upstream and downstream of the methionine in the SNCA mRNA sequence (i.e. start codon) as indicated.

In a particular embodiment, the target binding sequence is 40 nucleotides long and comprises, from 3′ to 5′ a sequence reverse complementary to 40 nucleotides of the 5′ UTR and 0 nucleotides of the CDS of the SNCA mRNA sequence. For example, the target binding sequence may comprise a sequence encoded by the DNA sequence of SEQ ID NO: 172 (i.e. −40/−1 of M1).

In a particular embodiment, the target binding sequence is 14 nucleotides long and comprises, from 3′ to 5′ a sequence reverse complementary to 14 nucleotides of the 5′ UTR and 0 nucleotides of the CDS of the SNCA mRNA sequence. For example, the target binding sequence may comprise a sequence encoded by the DNA sequence of SEQ ID NO: 174 (i.e. −14/−1 of M1).

In a particular embodiment, the target binding sequence is 14 nucleotides long and comprises, from 3′ to 5′ a sequence reverse complementary to 14 nucleotides of the 5′ UTR and 4 nucleotides of the CDS of the SNCA mRNA sequence. For example, the target binding sequence may comprise a sequence encoded by the DNA sequence of SEQ ID NO: 175 (i.e. −14/+4 of M1).

In one particular embodiment, the target binding sequence is 18 nucleotides long and comprises, from 3′ to 5′ a sequence reverse complementary to 14 nucleotides of the region upstream of an AUG site (start codon) of the SNCA mRNA and 4 nucleotides of the CDS of the SNCA mRNA sequence downstream of said AUG site, for example wherein the AUG site is methionine M2 (corresponding to M5 of the SNCA mRNA sequence). For example, the target binding sequence may comprise a sequence encoded by the DNA sequence of SEQ ID NO: 183 (i.e. −14/+4 of M2).

In another embodiment, the target binding sequence is 40 nucleotides long and comprises, from 3′ to 5′ a sequence reverse complementary to 40 nucleotides of the region upstream of an AUG site (start codon) of the SNCA mRNA and 0 nucleotides of the CDS of the SNCA mRNA sequence downstream of said AUG site, for example wherein the AUG site is methionine M2 (corresponding to M5 of the SNCA mRNA sequence). For example, the target binding sequence may comprise a sequence encoded by the DNA sequence of SEQ ID NO: 184 (i.e. −40/−1 of M2).

In a yet further example, human SNCA mRNA has 92.91% sequence identity across its entire length with Macaca mulatta SNCA mRNA and 83.73% sequence identity with mouse SNCA mRNA. Thus, in a yet further embodiment, the target binding sequence comprising or consisting of any of SEQ ID NOs: 168-190 binds Macaca mulatta SNCA and/or mouse SNCA.

In one embodiment, the target binding sequence comprises a sequence with at least 75% sequence identity, at least 80% sequence identity, at least 85% sequence identity, preferably at least 90% sequence identity, at least 91% sequence identity, at least 92% sequence identity, at least 93% sequence identity, at least 94% sequence identity, more preferably at least 95% sequence identity, at least 96% sequence identity, at least 97% sequence identity, at least 98% sequence identity, at least 99% sequence identity, even more preferably 100% sequence identity to any of SEQ ID NOs: 158-202, preferably SEQ ID NOs: 158-172, 174, 175, 177-179, 184, 186, 187, 195, 196, 200 or 201, in particular SEQ ID NOs: 158-172, 174, 175, 177-179.

In a further embodiment, the target binding sequence consists of a sequence with at least 75% sequence identity, at least 80% sequence identity, at least 85% sequence identity, preferably at least 90% sequence identity, at least 91% sequence identity, at least 92% sequence identity, at least 93% sequence identity, at least 94% sequence identity, more preferably at least 95% sequence identity, at least 96% sequence identity, at least 97% sequence identity, at least 98% sequence identity, at least 99% sequence identity, even more preferably 100% sequence identity to any of SEQ ID NOs: 158-202, preferably SEQ ID NOs: 158-172, 174, 175, 177-179, 184, 186, 187, 195, 196, 200 or 201, in particular SEQ ID NOs: 158-172, 174, 175, 177-179.

Animal models generated by methods of the invention may be used to screen potential therapeutic agents in the treatment of Parkinson's disease. The model may be used to determine the efficacy, toxicity, side effects, or mechanism of action, of treatment with such an agent. Once a PD disease state is established in an animal, an active substance or potential therapeutic agent may then be administered to the animal model. The protocol and route of administration will vary according to the mechanism of action and the chemical nature of the active substance and may be determined by those skilled in the art. Animals subjected to the potential therapeutic agent may be assessed by any appropriate method including behavioural, biochemical, and histological assessments, for any possible effects of the potential therapeutic agent and compared to control animals (e.g. PD animals that were not treated).

It will be understood that the embodiments described herein may be applied to all aspects of the invention, i.e. the embodiment described for the functional nucleic acid molecules may equally apply to the claimed methods and so forth.

### CLAUSES

1. A therapeutic agent for use in a method of treating a disease or disorder of the nervous system, wherein the therapeutic agent increases translation of an endogenous mRNA sequence selected from the group consisting of: an endogenous Nurr1, human GDNF, cRET or GBA mRNA sequence.
2. The therapeutic agent for use according to clause 1, wherein the disease or disorder of the nervous system is a disease or disorder of the central nervous system.
3. The therapeutic agent for use according to clause 1 or clause 2, wherein the disease or disorder of the nervous system is a neurodegenerative disease.
4. The therapeutic agent for use according to any one of clauses 1 to 3, wherein the therapeutic agent comprises a functional nucleic acid molecule comprising:
   at least one target binding sequence comprising a sequence reverse complementary to a target mRNA sequence selected from the group consisting of: a Nurr1, GDNF, cRET or GBA mRNA sequence; and
   at least one regulatory sequence comprising a SINE B2 element, a functionally active fragment of a SINE B2 element, an internal ribosome entry site (IRES) sequence or an IRES derived sequence.
5. The therapeutic agent for use according to clause 4, wherein the at least one target binding sequence comprises a sequence reverse complementary to a target mRNA sequence selected from the group consisting of: human Nurr1, human GDNF, human cRET or human GBA mRNA sequence.
6. The therapeutic agent for use according to clause 4, wherein the at least one target binding sequence comprises a sequence reverse complementary to a target mRNA sequence selected from the group consisting of: mouse Nurr1, mouse cRET or mouse GBA mRNA sequence.
7. The therapeutic agent for use according to any one of clauses 4 to 6, wherein the at least one target binding sequence comprises a sequence reverse complementary to a portion of the target mRNA sequence that is common to all isoforms.
8. The therapeutic agent for use according to any one of clauses 4 to 7, wherein the at least one target binding sequence is at least 18 nucleotides long and comprises, from 3′ to 5′:
   a sequence reverse complementary to 0 to 20 nucleotides of the 5′ UTR and 0 to 4 nucleotides of the CDS of the Nurr1 mRNA sequence; or
   a sequence reverse complementary to 0 to 18 nucleotides of the region upstream of an AUG site (start codon) of the Nurr1 mRNA and 0 to 4 nucleotides of the CDS of the Nurr1 mRNA sequence downstream of said AUG site.
9. The therapeutic agent for use according to any one of clauses 4 to 7, wherein the at least one target binding sequence is at least 14 nucleotides long and comprises, from 3′ to 5′:
   a sequence reverse complementary to 0 to 21 nucleotides of the 5′ UTR and 0 to 20 nucleotides of the CDS of the GDNF mRNA sequence; or
   a sequence reverse complementary to 0 to 20 nucleotides of the region upstream of an AUG site (start codon) of the GDNF mRNA and 0 to 4 nucleotides of the CDS of the GDNF mRNA sequence downstream of said AUG site.
10. The therapeutic agent for use according to any one of clauses 4 to 7, wherein the at least one target binding sequence is at least 15 nucleotides long and comprises, from 3′ to 5′:
   a sequence reverse complementary to 0 to 14 nucleotides of the 5′ UTR and 0 to 23 nucleotides of the CDS of the cRET mRNA sequence; or
   a sequence reverse complementary to 0 to 14 nucleotides of the region upstream of an AUG site (start codon) of the cRET mRNA and 0 to 13 nucleotides of the CDS of the cRET mRNA sequence downstream of said AUG site.
11. The therapeutic agent for use according to any one of clauses 4 to 7, wherein the at least one target binding sequence is at least 15 nucleotides long and comprises, from 3′ to 5′:
   a sequence reverse complementary to 0 to 56 nucleotides of the 5′ UTR and 0 to 14 nucleotides of the CDS of the GBA mRNA sequence; or
   a sequence reverse complementary to 0 to 40 nucleotides of the region upstream of an AUG site (start codon) of the GBA mRNA and 0 to 12 nucleotides of the CDS of the GBA mRNA sequence downstream of said AUG site.
12. The therapeutic agent for use according to any one of clauses 4 to 11, wherein the at least one regulatory sequence comprises a sequence with at least 75% sequence identity with a sequence selected from the group consisting of SEQ ID NOs: 1-69.
13. The therapeutic agent for use according to clause 12, wherein the at least one regulatory sequence comprises a sequence with at least 90% sequence identity with a sequence selected from the group consisting of SEQ ID NOs: 1-69.
14. The therapeutic agent for use according to any one of clauses 4 to 13, further comprising at least one linker sequence between the at least one target binding sequence and the at least one regulatory sequence.
15. The therapeutic agent for use according to any one of clauses 1 to 14, wherein the functional nucleic acid molecule is circular.
16. A functional nucleic acid molecule comprising:
   at least one target binding sequence comprising a sequence reverse complementary to a target mRNA sequence selected from the group consisting of: a Nurr1, GDNF, cRET or GBA mRNA sequence; and
   at least one regulatory sequence comprising a SINE B2 element, a functionally active fragment of a SINE B2 element, an IRES sequence or an IRES derived sequence.
17. A DNA molecule encoding the functional nucleic acid molecule according to clause 16.
18. An expression vector comprising the functional nucleic acid molecule according to clause 16 or the DNA molecule according to clause 17.
19. A composition comprising the functional nucleic acid molecule according to clause 16, the DNA molecule according to clause 17 or the expression vector according to clause 18.
20. A method for increasing the protein synthesis efficiency of Nurr1, GDNF, cRET or GBA in a cell comprising administering the functional nucleic acid molecule according clause 16, the DNA molecule according to clause 17 or the expression vector according to clause 18 to the cell.
21. The method according to clause 20, wherein the functional nucleic acid molecule is administered as naked RNA.
22. The method according to clause 20 or clause 21, wherein the cell is Nurr1, GDNF, CRET or GBA haploinsufficient.
23. The functional nucleic acid molecule clause 16, the DNA molecule according to clause 17 or the composition according to clause 19, for use as a medicament.
24. The functional nucleic acid molecule according clause 16, the DNA molecule according to clause 17 or the composition according to clause 19, for use in a method of treating a disease or disorder of the nervous system.
25. The functional nucleic acid molecule for use according to clause 24, wherein the disease or disorder of the nervous system is a disease or disorder of the central nervous system.
26. The functional nucleic acid molecule for use according to clause 24 or clause 25, wherein the disease or disorder of the nervous system is a neurodegenerative disorder.
27. The functional nucleic acid molecule for use according to clause 26, wherein the neurodegenerative disorder is Parkinson's disease.
28. A method of treating a disease or disorder of the nervous system comprising administering a therapeutic agent that increases translation of an endogenous mRNA sequence selected from the group consisting of: an endogenous Nurr1, human GDNF, cRET or GBA mRNA sequence.
29. A method of treating a disease or disorder of the nervous system comprising administering a therapeutically effective amount of the functional nucleic acid molecule according to clause 16, the DNA molecule according to clause 17 or the composition according to clause 19.
30 The method according to clause 28 or clause 29, wherein the disease or disorder of the nervous system is a disease or disorder of the central nervous system.
31. The method according to any one of clauses 28 to 30, wherein the disease or disorder of the nervous system is a neurodegenerative disorder.
32. The method according to clause 31, wherein the neurodegenerative disorder is Parkinson's disease.
33. The method according to any one of clauses 28 to 32, wherein the therapeutically effective amount is administered to the brain.
34. A method of producing a non-human animal model of Parkinson's disease comprising: administering a functional nucleic acid molecule comprising: at least one target binding sequence comprising a sequence reverse complementary to a SNCA mRNA sequence; and at least one regulatory sequence comprising a SINE B2 element, a functionally active fragment of a SINE B2 element, an IRES sequence or an IRES derived sequence, to a non-human animal.
35. The method according to clause 34, wherein the non-human animal is a non-human primate.

The invention will now be illustrated with reference to the following non-limiting examples.

### EXAMPLES

### Example 1

Binding domain sequences were designed to target human Nurr1, GDNF, cRET and GBA mRNA (summarised in Table 1) and their off-target binding (i.e. binding to mRNAs other than human Nurr1, GDNF, cRET or GBA mRNA) was analysed in silico using the UCSC browser (BLAT tool). An e-value greater than or equal to 2.1 represents low target specificity. The results are presented in Tables 2-5.

TABLE 1
Summary of designed binding domains for listed targets

| | | |
|---|---|---|
| Name | Binding domain | Methionine in target mRNA |

NURR1

| | | |
|---|---|---|
| hNURR1 BD1 | −14/+4 | M1 |
| hNURR1 BD2 | −18/+4 | M1 |
| hNURR1 BD3 | −18/−1 | M1 |
| hNURR1 BD4 | −20/−1 | M1 |
| hNURR1 BD5 | −20/+4 | M1 |
| hNURR1 BD6 | −14/+4 | M2 |
| hNURR1 BD7 | −18/−1 | M2 |
| hNURR1 BD8 | −18/+4 | M2 |
| hNURR1 BD9 | −14/+4 | M3 |

GDNF

| | | |
|---|---|---|
| hGDNF BD1 | −35/−18 | M1 |
| hGDNF BD2 | −40/−3 | M1 |
| hGDNF BD3 | −40/−1 | M1 |
| hGDNF BD4 | −26/−1 | M1 |
| hGDNF BD5 | −14/−1 | M1 |
| hGDNF BD6 | −14/+4 | M1 |
| hGDNF BD7 | −15/+4 | M1 |
| hGDNF BD8 | −16/+4 | M1 |
| hGDNF BD9 | −27/+4 | M1 |
| hGDNF BD10 | −40/+4 | M1 |
| hGDNF BD11 | −32/+8 | M1 |
| hGDNF BD12 | −56/+14 | M1 |
| hGDNF BD13 | −15/+16 | M1 |
| hGDNF BD14 | −20/+1 | M1 |
| hGDNF BD15 | −14/+4 | M2 |
| hGDNF BD16 | −25/−1 | M2 |
| hGDNF BD17 | −42/+4 | M3 |
| hGDNF BD18 | −14/+4 | M3 |
| hGDNF BD19 | +270/+288 | M4 |
| hGDNF BD20 | −21/+4 | M1 |
| hGDNF BD21 | −21/−1 | M1 |
| hGDNF BD22 | −1/+20 | M1 |
| hGDNF BD23 | −1/+17 | M1 |
| hGDNF BD24 | −14/−1 | M2 |
| hGDNF BD25 | −18/−1 | M2 |
| hGDNF BD26 | −20/+4 | M2 |

cRET

| | | |
|---|---|---|
| hRET BD1 | −69/−30 | M1 |
| hRET BD2 | −20/−3 | M1 |
| hRET BD3 | −32/−4 | M1 |
| hRET BD4 | −40/−3 | M1 |
| hRET BD5 | −40/−1 | M1 |
| hRET BD6 | −14/−1 | M1 |
| hRET BD7 | −14/+4 | M1 |
| hRET BD8 | −15/+4 | M1 |
| hRET BD9 | −16/+4 | M1 |
| hRET BD10 | −40/+4 | M1 |
| hRET BD11 | −32/+8 | M1 |
| hRET BD12 | −56/+14 | M1 |
| hRET BD13 | −14/+4 | M2 |
| hRET BD14 | −40/−1 | M2 |
| hRET BD15 | −14/+4 | M3 |
| hRET BD16 | −50/−1 | M3 |
| hRET BD17 | +64/+82 | M1 |
| hRET BD18 | +323/341 | M1 |
| hRET BD19 | +96/+114 | M2 |
| hRET BD20 | +65/+109 | M2 |
| hRET BD21 | −13/+5 | M1 |
| hRET BD22 | +82/+106 | M2 |
| hRET BD23 | −14/+3 | M1 |
| hRET BD24 | −11/+4 | M1 |
| hRET BD25 | −11/+7 | M1 |
| hRET BD26 | −11/+9 | M1 |
| hRET BD27 | −1/+17 | M1 |
| hRET BD28 | −1/+19 | M1 |
| hRET BD29 | −1/+23 | M1 |
| hRET BD30 | −14/+6 | M2 |
| hRET BD31 | −5/+13 | M2 |
| hRET BD32 | −4/+11 | M2 |

GBA

| | | |
|---|---|---|
| hGBA BD1 | −77/−59 | M1 |
| hGBA BD2 | −20/+3 | M1 |
| hGBA BD3 | −40/−3 | M1 |
| hGBA BD4 | −40/−1 | M1 |
| hGBA BD5 | −40/+4 | M1 |
| hGBA BD6 | −14/−1 | M1 |
| hGBA BD7 | −14/+4 | M1 |
| hGBA BD8 | −15/+4 | M1 |
| hGBA BD9 | −16/+4 | M1 |
| hGBA BD10 | −32/+8 | M1 |
| hGBA BD11 | −56/+14 | M1 |
| hGBA BD12 | −40/−1 | M2 |
| hGBA BD13 | −14/+4 | M3 |
| hGBA BD14 | −40/−1 | M3 |
| hGBA BD15 | −40/+14 | M3 |
| hGBA BD16 | −14/+4 | M9 |
| hGBA BD17 | +96/+114 | M6 |
| hGBA BD18 | −+91/+109 | M6 |
| hGBA BD19 | −6/+12 | M3 |
| hGBA BD20 | −22/+8 | M6 |

TABLE 2
In silico off-target binding analysis
of Nurr1 target binding sequences/BDs

| Binding | | | | | Match |
|---|---|---|---|---|---|
| Domain | Gene | Length | E-val | % ID | Location |
| hNURR1 BD1 | NR4A2 | 18 | 0.009 | 100.00 | |
| hNURR1 BD2 | NR4A2 | 22 | 1.00E−04 | 100.00 | |
| | NDRG1 | 15 | 1.6 | 100.00 | Intron |
| hNURR1 BD3 | NR4A2 | 18 | 0.009 | 100.00 | |
| | NDRG1 | 15 | 0.54 | 100.00 | Intron |
| hNURR1 BD4 | NR4A2 | 20 | 0.001 | 100.00 | |
| | NDRG1 | 15 | 1.1 | 100.00 | Intron |
| hNURR1 BD5 | NR4A2 | 24 | 8.00E−06 | 100.00 | |
| | NDRG1 | 15 | 1.9 | 100.00 | Intron |
| hNURR1 BD6 | NR4A2 | 18 | 0.009 | 100.00 | |
| | NANOG | 15 | 0.54 | 100.00 | 3′UTR |
| hNURR1 BD7 | NR4A2 | 18 | 0.009 | 100.00 | |
| hNURR1 BD8 | NR4A2 | 22 | 1.00E−04 | 100.00 | |
| | GCN1 | 20 | 0.41 | 95.00 | 3′UTR |
| | NANOG | 15 | 1.6 | 100.00 | 3′UTR |
| hNURR1 BD9 | NR4A2 | 18 | 0.009 | 100.00 | |
| | AC090142.3 | 15 | 0.54 | 100.00 | Processed |
| | | | | | pseudo- |
| | | | | | gene |
| hNURR1 | NR4A2 | 18 | 0.009 | 100.00 | |
| BD10 | DCHS1 | 15 | 0.54 | 100.00 | Exon 14 |
| | | | | | of 21 |

TABLE 3
In silico off-target binding analysis
of GDNF target binding sequences/BDs

| Binding | | | | | Match |
|---|---|---|---|---|---|
| Domain | Gene | Length | E-val | % ID | Location |
| hGDNF BD1 | GDNF | 18 | 0.009 | 100.00 | |
| hGDNF BD2 | GDNF | 38 | 1.00E−13 | 100.00 | |
| hGDNF BD3 | GDNF | 40 | 8.00E−15 | 100.00 | |
| hGDNF BD4 | GDNF | 26 | 7.00E−07 | 100.00 | |
| hGDNF BD5 | GDNF | 14 | 2.1 | 100.00 | |
| hGDNF BD6 | GDNF | 18 | 0.009 | 100.00 | |
| hGDNF BD7 | GDNF | 19 | 0.003 | 100.00 | |
| hGDNF BD8 | GDNF | 20 | 0.001 | 100.00 | |
| hGDNF BD9 | GDNF | 31 | 1.00E−09 | 100.00 | |
| hGDNF BD10 | GDNF | 44 | 4.00E−17 | 100.00 | |
| hGDNF BD11 | GDNF | 40 | 8.00E−15 | 100.00 | |
| hGDNF BD12 | GDNF | 70 | 3.00E−32 | 100.00 | |
| hGDNF BD13 | GDNF | 31 | 1.00E−09 | 100.00 | |
| hGDNF BD14 | GDNF | 21 | 4.00E−04 | 100.00 | |
| hGDNF BD15 | GDNF | 18 | 0.009 | 100.00 | |
| | ZNF785 | 15 | 0.54 | 100.00 | 3′ UTR |
| hGDNF BD16 | GDNF | 25 | 2.00E−06 | 100.00 | |
| hGDNF BD17 | GDNF | 46 | 3.00E−18 | 100.00 | |
| hGDNF BD18 | GDNF | 18 | 0.009 | 100.00 | |
| hGDNF BD19 | GDNF | 18 | 0.009 | 100.00 | |
| | CLVS1 | 15 | 0.54 | 100.00 | Retained |
| | | | | | intron |
| | LAMTOR3 | 15 | 0.54 | 100.00 | 3′ UTR |
| | PRCD | 15 | 0.54 | 100.00 | 3′ UTR |
| hGDNF BD20 | GDNF | 25 | 2.00E−06 | 100.00 | |
| hGDNF BD21 | GDNF | 21 | 4.00E−04 | 100.00 | |
| hGDNF BD22 | GDNF | 21 | 4.00E−04 | 100.00 | |
| hGDNF BD23 | GDNF | 18 | 0.009 | 100.00 | |
| hGDNF BD24 | GDNF | 14 | 2.1 | 100.00 | |
| hGDNF BD25 | GDNF | 18 | 0.009 | 100.00 | |
| | RNF207 | 15 | 0.54 | 100.00 | 3′ UTR |
| hGDNF BD26 | GDNF | 24 | 8.00E−06 | 100.00 | |
| | RNF207 | 15 | 1.9 | 100.00 | 3′ UTR |
| | ZNF785 | 15 | 1.9 | 100.00 | 5′ UTR |
| | OR8B8 | 19 | 1.9 | 94.74 | 3′ UTR |

TABLE 4
In silico off-target binding analysis
of cRET target binding sequences/BDs

| Binding | | | | | Match |
|---|---|---|---|---|---|
| Domain | Gene | Length | E-val | % ID | Location |
| hRET BD1 | RET | 40 | 8.00E−15 | 100.00 | |
| | CKBP1 | 16 | 1.6 | 100.00 | Processed |
| | | | | | pseudogene |
| hRET BD2 | RET | 18 | 0.009 | 100.00 | |
| hRET BD3 | RET | 29 | 2.00E−08 | 100.00 | |
| | DNM1P51 | 17 | 0.21 | 100.00 | Processed |
| | | | | | pseudogene |
| | ABCD1P4 | 16 | 0.82 | 100.00 | Unprocessed |
| | | | | | pseudogene |
| | ABCD1P5 | 16 | 0.82 | 100.00 | Unprocessed |
| | | | | | pseudogene |
| | ABCD1P2 | 16 | 0.82 | 100.00 | Unprocessed |
| | | | | | pseudogene |
| | ABCD1 | 16 | 0.82 | 100.00 | Exon10 |
| | | | | | of 10 |
| hRET BD4 | RET | 38 | 1.00E−13 | 100.00 | |
| | DNM1P51 | 17 | 0.37 | 100.00 | Processed |
| | | | | | pseudogene |
| | ABCD1P4 | 16 | 1.4 | 100.00 | Unprocessed |
| | | | | | pseudogene |
| | ABCD1P5 | 16 | 1.4 | 100.00 | Unprocessed |
| | | | | | pseudogene |
| | ABCD1P2 | 16 | 1.4 | 100.00 | Unprocessed |
| | | | | | pseudogene |
| | ABCD1 | 16 | 1.4 | 100.00 | Exon10 |
| | | | | | of 10 |
| | KIAA1614 | 16 | 1.4 | 100.00 | 3′ UTR |
| hRET BD5 | RET | 40 | 8.00E−15 | 100.00 | |
| | DNM1P51 | 17 | 0.40 | 100.00 | Processed |
| | | | | | pseudogene |
| | ABCD1P2 | 16 | 1.6 | 100.00 | Unprocessed |
| | | | | | pseudogene |
| | ABCD1P4 | 16 | 1.6 | 100.00 | Unprocessed |
| | | | | | pseudogene |
| | ABCD1P5 | 16 | 1.6 | 100.00 | Unprocessed |
| | | | | | pseudogene |
| | ABCD1 | 16 | 1.6 | 100.00 | Exon10 |
| | | | | | of 10 |
| | KIAA1614 | 16 | 1.6 | 100.00 | 3′ UTR |
| hRET BD6 | NRG2 | 14 | 2.1 | 100.00 | Exon8 of 8 |
| | LRRC75A | 14 | 2.1 | 100.00 | M1 |
| | | | | | −74/−86 |
| | RET | 14 | 2.1 | 100.00 | |
| hRET BD7 | RET | 18 | 0.009 | 100.00 | |
| hRET BD8 | RET | 19 | 0.003 | 100.00 | |
| | NRG2 | 15 | 0.81 | 100.00 | Exon8 of 8 |
| hRET BD9 | RET | 20 | 0.001 | 100.00 | |
| | NRG2 | 15 | 1.1 | 100.00 | |
| hRET BD10 | RET | 44 | 4.00E−17 | 100.00 | |
| | DNM1P51 | 17 | 0.45 | 100.00 | Processed |
| | | | | | pseudogene |
| | ABCD1P5 | 16 | 1.8 | 100.00 | Unprocessed |
| | | | | | pseudogene |
| | ABCD1P2 | 16 | 1.8 | 100.00 | Unprocessed |
| | | | | | pseudogene |
| | ABCD1 | 16 | 1.8 | 100.00 | Exon10 |
| | | | | | of 10 |
| | KIAA1614 | 16 | 1.8 | 100.00 | 3′ UTR |
| hRET BD11 | RET | 40 | 8.00E−15 | 100.00 | |
| | DNM1P51 | 17 | 0.40 | 100.00 | Processed |
| | | | | | pseudogene |
| | ABCD1P5 | 16 | 1.6 | 100.00 | Unprocessed |
| | | | | | pseudogene |
| | ABCD1P2 | 16 | 1.6 | 100.00 | Unprocessed |
| | | | | | pseudogene |
| | ABCD1 | 16 | 1.6 | 100.00 | Exon10 |
| | | | | | of 10 |
| hRET BD12 | RET | 70 | 3.00E−32 | 100.00 | |
| | DNM1P51 | 17 | 0.91 | 100.00 | Unprocessed |
| | | | | | pseudogene |
| hRET BD13 | RET | 18 | 0.009 | 100.00 | |
| | S1PR3 | 16 | 0.14 | 100.00 | 3′ UTR |
| | LRCH4 | 16 | 0.14 | 100.00 | Intron |
| | ZDHHC3 | 15 | 0.54 | 100.00 | 3′ UTR |
| | ARPIN- | 15 | 0.54 | 100.00 | Exon9 |
| | AP3S2 | | | | of 10 = |
| | | | | | M329/M326 |
| | AP3S2 | 15 | 0.54 | 100.00 | Exon5 of 6 - |
| | | | | | M128/M125 |
| hRET BD14 | RET | 40 | 8.00E−15 | 100.00 | |
| | CCDC102A | 17 | 0.40 | 100.00 | M94 |
| | | | | | +63/+84 |
| | YY1 | 17 | 0.40 | 100.00 | M83 |
| | | | | | +73/+95 |
| hRET BD15 | RET | 18 | 0.009 | 100.00 | |
| hRET BD16 | RET | 50 | 1.00E−20 | 100.00 | |
| hRET BD17 | RET | 18 | 0.009 | 100.00 | |
| | SLC30A7 | 15 | 0.54 | 100.00 | 3′ UTR |
| hRET BD18 | RET | 18 | 0.009 | 100.00 | |
| hRET BD19 | RET | 18 | 0.009 | 100.00 | |
| | ZCCHC2 | 15 | 0.54 | 100.00 | M1 |
| | | | | | −304/−286 |
| hRET BD20 | RET | 44 | 4.00E−17 | 100.00 | |
| | SLC6A1 | 20 | 1.8 | 95.00 | Exon4 |
| | | | | | of 14 - |
| | | | | | M7 +1/+21 |
| hRET BD21 | RET | 18 | 0.009 | 100.00 | |
| | AC020765.5 | 15 | 0.54 | 100.00 | Unprocessed |
| | | | | | pseudogene |
| | AC020765.3 | 15 | 0.54 | 100.00 | Unprocessed |
| | | | | | pseudogene |
| | SULT1A1 | 15 | 0.54 | 100.00 | Intron |
| hRET BD22 | RET | 24 | 8.00E−06 | 100.00 | |
| hRET BD23 | RET | 17 | 0.035 | 100.00 | |
| hRET BD24 | RET | 15 | 0.54 | 100.00 | |
| hRET BD25 | RET | 18 | 0.009 | 100.00 | |
| | AC020765.5 | 15 | 0.54 | 100.00 | Unprocessed |
| | | | | | pseudogene |
| | SULT1A1 | 15 | 0.54 | 100.00 | Intron |
| hRET BD26 | RET | 20 | 0.001 | 100.00 | |
| | AC020765.5 | 15 | 1.1 | 100.00 | Processed |
| | | | | | pseudogene |
| | SULT1A1 | 15 | 1.1 | 100.00 | Intron |
| hRET BD27 | RET | 18 | 0.009 | 100.00 | |
| hRET BD28 | RET | 20 | 0.001 | 100.00 | |
| hRET BD29 | RET | 24 | 8.00E−06 | 100.00 | |
| hRET BD30 | RET | 20 | 0.001 | 100.00 | |
| | S1PR3 | 18 | 0.018 | 100.00 | 3′UTR |
| | ZDHHC3 | 17 | 0.070 | 100.00 | 3′UTR |
| | LRCH4 | 16 | 0.27 | 100.00 | Intron |
| | SNX13 | 15 | 1.1 | 100.00 | Intron |
| | ARPIN- | 15 | 1.1 | 100.00 | Exon5 of 6 |
| | AP3S2 | | | | |
| hRET BD31 | RET | 18 | 0.009 | 100.00 | |
| hRET BD32 | RET | 15 | 0.54 | 100.00 | |

TABLE 5
In silico off-target binding analysis
of GBA target binding sequences/BDs

| Binding | | | | | Match |
|---|---|---|---|---|---|
| Domain | Gene | Length | E-val | % ID | Location |
| hGBA BD1 | GBA | 18 | 0.009 | 100.00 | |
| | BEND4 | 16 | 0.14 | 100.00 | Exon2 of 6 |
| hGBA BD2 | GBA | 18 | 0.009 | 100.00 | |
| hGBA BD3 | GBA | 38 | 1.00E−13 | 100.00 | |
| | GBAP1 | 38 | 2.00E−06 | 92.11 | Unprocessed |
| | | | | | pseudogene |
| | OR11J7P | 21 | 1.4 | 95.24 | Unprocessed |
| | | | | | pseudogene |
| | AC183088.2 | 21 | 1.4 | 95.24 | Unprocessed |
| | | | | | pseudogene |
| | OR11J2P | 21 | 1.4 | 95.24 | Unprocessed |
| | | | | | pseudogene |
| hGBA BD4 | GBA | 40 | 8.00E−15 | 100.00 | |
| | GBAP1 | 38 | 2.00E−06 | 92.11 | Unprocessed |
| | | | | | pseudogene |
| | OR11J7P | 21 | 1.6 | 95.24 | Unprocessed |
| | | | | | pseudogene |
| | AC183088.2 | 21 | 1.6 | 95.24 | Unprocessed |
| | | | | | pseudogene |
| | OR11J2P | 21 | 1.6 | 95.24 | Unprocessed |
| | | | | | pseudogene |
| hGBA BD5 | GBA | 44 | 4.00E−17 | 100.00 | |
| | GBAP1 | 44 | 1.00E−07 | 90.91 | Unprocessed |
| | | | | | pseudogene |
| | OR11J7P | 21 | 1.8 | 95.24 | Unprocessed |
| | | | | | pseudogene |
| | AC183088.2 | 21 | 1.8 | 95.24 | Unprocessed |
| | | | | | pseudogene |
| | OR11J2P | 21 | 1.8 | 95.24 | Unprocessed |
| | | | | | pseudogene |
| hGBA BD6 | GBA | 14 | 2.1 | 100.00 | |
| hGBA BD7 | GBA | 18 | 0.009 | 100.00 | |
| | NDST1 | 15 | 0.54 | 100.00 | 3′ UTR |
| hGBA BD8 | GBA | 19 | 0.003 | 100.00 | |
| | NDST1 | 15 | 0.81 | 100.00 | 3′ UTR |
| hGBA BD9 | GBA | 20 | 0.001 | 100.00 | |
| | NDST1 | 15 | 1.1 | 100.00 | 3′ UTR |
| hGBA BD10 | GBA | 40 | 8.00E−15 | 100.00 | |
| | GBAP1 | 37 | 7.00E−06 | 91.89 | Unprocessed |
| | | | | | pseudogene |
| hGBA BD11 | GBA | 70 | 3.00E−32 | 100.00 | |
| | GBAP1 | 70 | 9.00E−23 | 94.29 | |
| hGBA BD12 | GBA | 40 | 8.00E−15 | 100.00 | |
| | GBAP1 | 40 | 5.00E−10 | 95.00 | Unprocessed |
| | | | | | pseudogene |
| | NPHP1 | 16 | 1.6 | 100.00 | 3′ UTR |
| hGBA BD13 | GBA | 18 | 0.009 | 100.00 | |
| hGBA BD14 | GBA | 40 | 8.00E−15 | 100.00 | |
| hGBA BD15 | GBA | 54 | 6.00E−23 | 100.00 | |
| | GBAP1 | 51 | 0.040 | 84.31 | Unprocessed |
| | | | | | pseudogene |
| hGBA BD16 | GBA | 18 | 0.009 | 100.00 | |
| hGBA BD17 | GBA | 18 | 0.009 | 100.00 | |
| | GBAP1 | 18 | 0.009 | 100.00 | Unprocessed |
| | | | | | pseudogene |
| | MKI67 | 16 | 0.14 | 100.00 | Exon13 of |
| | | | | | 15 - M2131 |
| hGBA BD18 | GBA | 18 | 0.009 | 100.00 | |
| | DGKA | 15 | 0.54 | 100.00 | Exon21 of |
| | | | | | 24 - M665 |
| | SRP9 | 15 | 0.54 | 100.00 | Exon3 of 3 - |
| | | | | | M52 |
| hGBA BD19 | GBA | 18 | 0.009 | 100.00 | |
| hGBA BD20 | GBA | 30 | 4.00E−09 | 100.00 | |
| | AC217785.3 | 30 | 1.00E−06 | 96.67 | Processed |
| | | | | | pseudogene |
| | GBAP1 | 30 | 1.00E−06 | 96.67 | Unprocessed |
| | | | | | pseudogene |

Although a number of putative off-target binding targets have been identified for the disclosed binding domains/target binding sequences disclosed herein, the pairing position is not significant for these, based on the binding position, AUG start codon/translation start site (TSS) and in-frame methionine. Therefore the designed BDs present high specificity since relevant off-targets have not been identified.

### Example 2

Binding domain sequences were designed to target mouse and human SNCA mRNA (summarised in Table 7) and the off-target binding (i.e. binding to mRNAs other than human SNCA mRNA) was analysed in silico using the UCSC browser (BLAT tool). An e-value greater than or equal to 2.1 represents low target specificity. The results are presented in Table 8.

TABLE 7
Summary of designed binding domains for SNCA

| | | |
|---|---|---|
| Name | Binding domain | Methionine in target mRNA |

Mouse SNCA (mSNCA) sequences

| | | |
|---|---|---|
| mSNCA BD1 | −35/−18 | M1 |
| mSNCA BD2 | −40/−3 | M1 |
| mSNCA BD3 | −41/−12 | M1 |
| mSNCA BD4 | −20/−3 | M1 |
| mSNCA BD5 | −40/−1 | M1 |
| mSNCA BD6 | −26/−1 | M1 |
| mSNCA BD7 | −14/−1 | M1 |
| mSNCA BD8 | −14/+4 | M1 |
| mSNCA BD9 | −15/+4 | M1 |
| mSNCA BD10 | −16+4 | M1 |
| mSNCA BD11 | −40/+4 | M1 |
| mSNCA BD12 | −32/+8 | M1 |
| mSNCA BD13 | −54/+16 | M1 |
| mSNCA BD14 | −26/+16 | M1 |
| mSNCA BD15 | −20/+1 | M1 |
| mSNCA BD16 | −14/+4 | M2 |
| mSNCA BD17 | −40/−1 | M2 |
| mSNCA BD18 | −1/+18 | M3 |
| mSNCA BD19 | −14/+23 | M3 |
| mSNCA BD20 | −42/+4 | M5 |
| mSNCA BD21 | +100/+118 | M2 |
| mSNCA BD22 | +109/+153 | M2 |

Human SNCA (hSNCA) sequences

| | | |
|---|---|---|
| hSNCA BD1 | −37/−17 | M1 |
| hSNCA BD2 | −40/−3 | M1 |
| hSNCA BD3 | −40/−11 | M1 |
| hSNCA BD4 | −20/−3 | M1 |
| hSNCA BD5 | −40/−1 | M1 |
| hSNCA BD6 | −26/−1 | M1 |
| hSNCA BD7 | −14/−1 | M1 |
| hSNCA BD8 | −14/+4 | M1 |
| hSNCA BD9 | −15/+4 | M1 |
| hSNCA BD10 | −16/+4 | M1 |
| hSNCA BD11 | −40/+4 | M1 |
| hSNCA BD12 | −32/+8 | M1 |
| hSNCA BD13 | −54/+16 | M1 |
| hSNCA BD14 | −25/+16 | M1 |
| hSNCA BD15 | −20/+1 | M1 |
| hSNCA BD16 | −14/+4 | M2 |
| hSNCA BD17 | −40/−1 | M2 |
| hSNCA BD18 | −9/+37 | M3 |
| hSNCA BD19 | −14/+4 | M3 |
| hSNCA BD20 | +100/+118 | M2 |
| hSNCA BD21 | +69/+113 | M2 |
| hSNCA BD22 | +285/+303 | M2 |
| hSNCA BD23 | −6/+12 | M1 |

TABLE 8
In silico off-target binding analysis of SNCA target binding sequences/BDs

| | | | | | |
|---|---|---|---|---|---|
| Binding Domain | Gene | Length | E-val | % ID | Match Location |

Mouse SNCA (mSNCA) sequences

| | | | | | |
|---|---|---|---|---|---|
| mSNCA BD1 | SNCA | 18 | 0.009 | 100.00 | |
| | DNAJC11 | 16 | 0.14 | 100.00 | Retained intron |
| mSNCA BD2 | SNCA | 38 | 8.00E−14 | 100.00 | |
| | DNAJC11 | 16 | 0.96 | 100.00 | Retained intron |
| mSNCA BD3 | SNCA | 30 | 3.00E−09 | 100.00 | |
| | DNAJC11 | 16 | 0.60 | 100.00 | Retained intron |
| mSNCA BD4 | SNCA | 18 | 0.009 | 100.00 | |
| | GRAP2 | 15 | 0.54 | 100.00 | 3′ UTR |
| | SGMS2 | 15 | 0.54 | 100.00 | 3′ UTR |
| mSNCA BD5 | SNCA | 40 | 6.00E−15 | 100.00 | |
| | DNAJC11 | 16 | 1.1 | 100.00 | Retained intron |
| mSNCA BD6 | SNCA | 27 | 1.00E−07 | 100.00 | |
| mSNCA BD7 | SNCA | 14 | 1.4 | 100.00 | |
| | RD3 | 14 | 1.4 | 100.00 | 3′ UTR |
| mSNCA BD8 | SNCA | 18 | 0.009 | 100.00 | |
| | VMN1R168 | 15 | 0.54 | 100.00 | 3′ UTR |
| | PPOX | 15 | 0.54 | 100.00 | Exon 4 of 9 |
| mSNCA BD9 | SNCA | 19 | 0.002 | 100.00 | |
| | VMN1R168 | 15 | 0.54 | 100.00 | 3′ UTR |
| | PPOX | 15 | 0.54 | 100.00 | Exon 4 of 9 |
| mSNCA BD10 | SNCA | 20 | 8.00E−04 | 100.00 | |
| | VMN1R168 | 15 | 0.72 | 100.00 | 3′ UTR |
| | PPOX | 15 | 0.72 | 100.00 | Exon 4 of 9 |
| mSNCA BD11 | SNCA | 44 | 3.00E−17 | 100.00 | |
| | DNAJC11 | 16 | 1.2 | 100.00 | Retained intron |
| mSNCA BD12 | SNCA | 40 | 6.00E−15 | 100.00 | |
| | PPOX | 16 | 1.1 | 100.00 | Exon 4 of 9 |
| mSNCA BD13 | SNCA | 42 | 4.00E−16 | 100.00 | |
| | PPOX | 16 | 1.1 | 100.00 | Exon 4 of 9 |
| mSNCA BD14 | SNCA | 42 | 4.00E−16 | 100.00 | |
| | PPOX | 16 | 1.1 | 100.00 | Exon 4 of 9 |
| mSNCA BD15 | SNCA | 21 | 2.00E−04 | 100.00 | |
| | GRAP2 | 15 | 0.90 | 100.00 | 3′ UTR |
| | SGMS2 | 15 | 0.90 | 100.00 | 3′ UTR |
| mSNCA BD16 | SNCA | 18 | 0.009 | 100.00 | |
| | VMN2R-PS87 | 15 | 0.54 | 100.00 | Processed transcript |
| | VMN2R77 | 15 | 0.54 | 100.00 | −96/−57 M1 |
| mSNCA BD17 | SNCA | 40 | 6.00E−15 | 100.00 | |
| | PPOX | 16 | 1.1 | 100.00 | Exon 4 of 9 |
| mSNCA BD18 | SNCA | 18 | 0.009 | 100.00 | |
| | MAPK11 | 15 | 0.54 | 100.00 | Intron |
| | ARHGAP4 | 15 | 0.54 | 100.00 | 3′ UTR |
| mSNCA BD19 | SNCA | 37 | 3.00E−13 | 100.00 | |
| | LGFBP2 | 16 | 0.92 | 100.00 | Exon 2 or 4 M36 |
| | WFDC8 | 16 | 0.92 | 100.00 | M1 |
| | STAC3 | 16 | 0.92 | 100.00 | Exon 11-12 of 12 |
| mSNCA BD20 | SNCA | 46 | 2.00E−18 | 100.00 | |
| | GM18814 | 17 | 0.34 | 100.00 | Processed pseudogene |
| | ING5 | 17 | 0.34 | 100.00 | Exon 6 of 8 |
| | WDSUB1 | 21 | 0.34 | 95.24 | Intron |
| | IKBKE | 16 | 1.3 | 100.00 | Intron |
| | SLC18A1 | 16 | 1.3 | 100.00 | Exon 17 or 17 |
| mSNCA BD21 | SNCA | 18 | 0.009 | 100.00 | |
| mSNCA BD22 | SNCA | 44 | 3.00E−17 | 100.00 | |
| | ASNS | 16 | 1.2 | 100.00 | Exon 12 of 12 |

Human SNCA (hSNCA) sequences

| | | | | | |
|---|---|---|---|---|---|
| hSNCA BD1 | SNCA | 18 | 0.009 | 100.00 | |
| hSNCA BD2 | SNCA | 38 | 1.00E−13 | 100.00 | |
| hSNCA BD3 | SNCA | 30 | 4.00E−09 | 100.00 | |
| hSNCA BD4 | SNCA | 18 | 0.009 | 100.00 | |
| hSNCA BD5 | SNCA | 40 | 8.00E−15 | 100.00 | |
| hSNCA BD6 | SNCA | 26 | 7.00E−07 | 100.00 | |
| hSNCA BD7 | SNCA | 14 | 2.2 | 100.00 | |
| hSNCA BD8 | SNCA | 18 | 0.009 | 100.00 | |
| hSNCA BD9 | SNCA | 20 | 0.001 | 100.00 | |
| hSNCA BD10 | SNCA | 20 | 0.001 | 100.00 | |
| hSNCA BD11 | SNCA | 44 | 4.00E−17 | 100.00 | |
| hSNCA BD12 | SNCA | 40 | 8.00E−15 | 100.00 | |
| hSNCA BD13 | SNCA | 70 | 3.00E−32 | 100.00 | |
| hSNCA BD14 | SNCA | 41 | 2.00E−15 | 100.00 | |
| hSNCA BD15 | SNCA | 21 | 4.00E−04 | 100.00 | |
| hSNCA BD16 | SNCA | 18 | 0.009 | 100.00 | |
| | AC020915.3 | 15 | 0.55 | 100.00 | Processed pseudogene |
| | QSOX2 | 15 | 0.55 | 100.00 | 3′ UTR |
| hSNCA BD17 | SNCA | 40 | 8.00E−15 | 100.00 | |
| hSNCA BD18 | SNCA | 46 | 3.00E−18 | 100.00 | |
| | ING4 | 17 | 0.50 | 100.00 | Exon6 of 8 - M189 |
| hSNCA BD19 | SNCA | 18 | 0.009 | 100.00 | |
| | NUP160 | 15 | 0.55 | 100.00 | No matches found |
| hSNCA BD20 | SNCA | 18 | 0.009 | 100.00 | |
| hSNCA BD21 | SNCA | 44 | 4.00E−17 | 100.00 | |
| | NUP85 | 16 | 1.8 | 100.00 | Intron |
| | ECPAS | 16 | 1.8 | 100.00 | No matches found |
| hSNCA BD22 | SNCA | 18 | 0.009 | 100.00 | |
| | ALDH7A1 | 16 | 0.14 | 100.00 | |
| | NHLH2 | 15 | 0.55 | 100.00 | 5′ UTR |
| hSNCA BD23 | SNCA | 18 | 0.009 | 100.00 | |

### Example 3

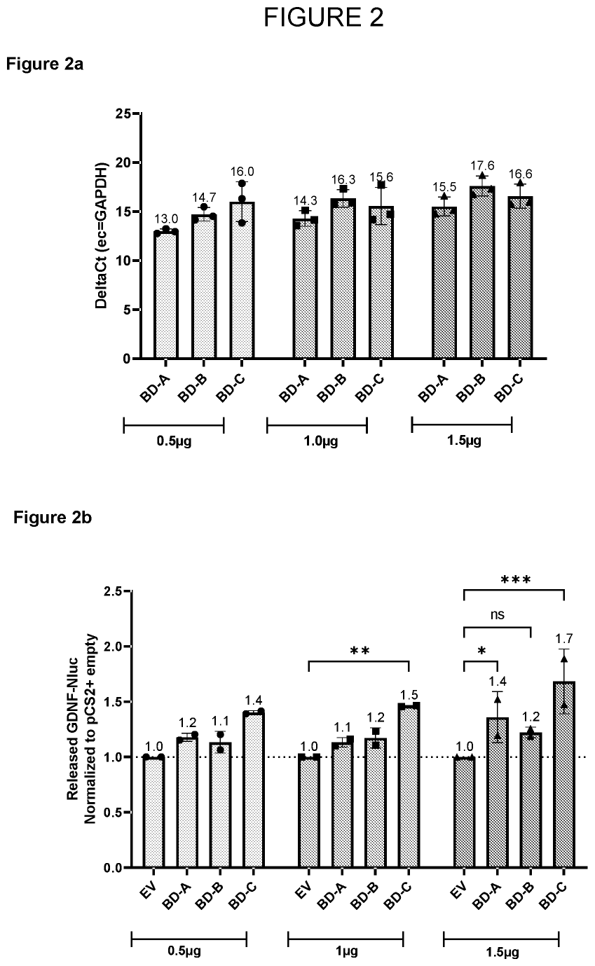

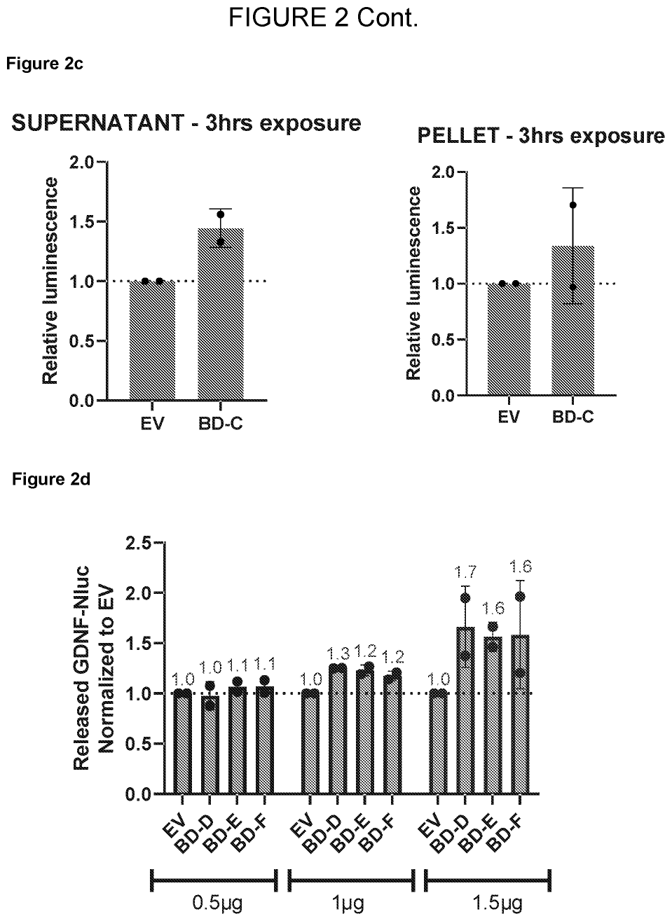

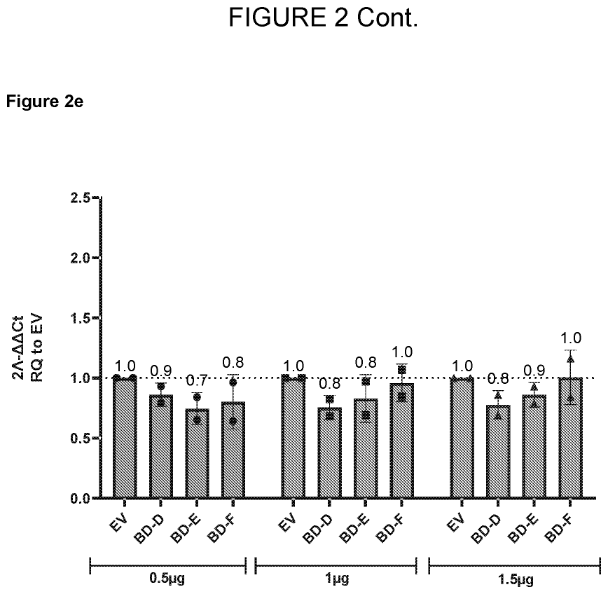

Human GDNF targeting (FIG. 2): A HEK293 cell line stably expressing human GDNF-nanoLuciferase (GDNF-nLuc) fusion protein was used for the analysis of GDNF SINEUP activity on target expression profile (the HEK-GDNF-nLuc model). The GDNF-nLuc construct contains the natural human 5′UTR and the coding sequence of the major GDNF transcript (variant 1, NM000514.4). This construct contains the 5′ sequence that would be A HEK293 cell line stably expressing human GDNF-nanoLuciferase (GDNF-nLuc) fusion protein was used for the analysis of GDNF SINEUP activity on target expression profile (the HEK-GDNF-nLuc model). The GDNF-nLuc construct contains the natural human 5′UTR and the coding sequence of the major GDNF transcript (variant 1, NM000514.4). This construct contains the 5′ sequence that would be recognised by all of the human GDNF (hGDNF) SINEUPs. Western blot analysis demonstrated that either GDNF or nLuc can be detected to quantify GDNF-nLuc fusion protein expression. GDNF-nLuc can be detected using a human GDNF ELISA. Detection of hGDNF-nLuc using luminescence mirrors detection of GDNF-nLuc with ELISA. These data demonstrates that luciferase acts as a marker for GDNF protein expression and secretion, i.e. secreted luciferase=secreted GDNF.

In the first experimental series, HEK-GDNF-nLuc cells were seeded in 6-well plates, 24 hrs prior to transfection. Three SINEUPs with the micro effector domain (ED) were tested: BD-A (−14/+4), BD-B (−40/+4) and BD-C (−56/+14) binding domains (BDs) for comparative testing. Three SINEUPs with the miniED, which differ in BD length, were also tested: BD-D (−14/+4), BD-E (−14/+4) and BD-F (−8/+17). The SINEUPs were transfected using Lipofectamine according to manufacturer's instructions for 48 hrs at three DNA concentrations (0.5 ug, 1 ug and 1.5 ug/well). After 48 hrs of expression, the media was replaced and samples were collected for analysis after further incubation for 1 hr. Secreted nLuc was used as a surrogate for secreted GDNF-nLuc and was detected using the NanoGlo Luciferase Assay kit (Promega). Data are expressed as fold increase over empty control vector (EV) and a 2-way Anova was applied. RT-qPCR was undertaken to assess the expression profiles of SINEUP and target GDNF mRNA. For expression of GDNF mRNA, data is plotted as fold increase relative to EV. For SINEUP RNA data Ct values normalized to endogenous control are plotted. N=2.

In the second experimental series (the transport inhibitor experiment), 150,000 HEK-GDNF-nLuc cells per well were seeded on 24-well plates and, 24 hours later, transfected with 400 ng of either EV or BD-C (−56/+14) using Lipofectamine 3000 reagents according to manufacturer's instructions. 24 hrs after transfection, cells were treated with either protein inhibitor cocktail (Brefeldin A and Monesin, (eBioscience Protein Transport Inhibitor cocktail (500×)) at a 1× final concentration or 100% EtOH (vehicle) for 3 hours. Supernatants (1/10 volume/sample) and pellets (⅓ of final mass/sample) were collected and analyzed separately for luminescence using the NanoGlo Luciferase Assay kit (Promega). For each inhibitor-treated sample, luminescence values (all normalized to protein concentration) were first normalized to the matching EtOH control. To evaluate SINEUP effect, normalized values of BD-C-transfected samples were further normalized to EV-transfected samples and plotted; n=2.

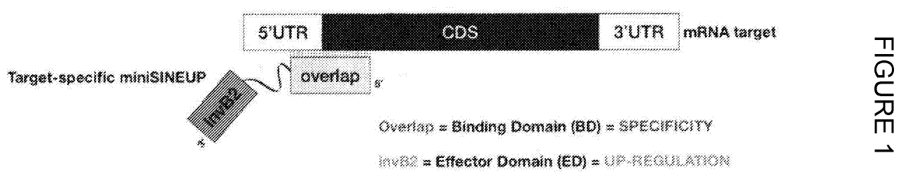

Preliminary data demonstrating a positive effect of hGDNF SINEUPs on hGDNF-nLuc protein expression were generated using the HEK GDNF-nLuc model system. Exemplary data are shown in FIG. 2. As determined by RT-qPCR, the SINEUPs were abundantly expressed in the HEK-GDNF-nLuc cell line. Data showing microSINEUP expression is shown in FIG. 1a. Concentration-dependent hGDNF microSINEUP RNA expression was observed with increasing plasmid concentration up to 1.5 ug of DNA for all SINEUPs tested (data not shown). HEK-GDNF-nLuc cells expressing microSINEUPs displayed elevated levels of secreted nLuc levels, when compared to EV alone (FIG. 2b). Discrimination between SINEUPs was observed: BD-C (−56/+14) elevates secreted nLuc levels to a greater extent than BD-A (−14/+4) and BD-B (−40/+4) In addition, a SINEUP effect was also observed in the transport blocker experiment, where BD-C (−56/+14) elevated the level of both secreted and intracellular luciferase compared to cells transfected with empty vector (EV) (FIG. 2c). HEK-GDNF-nLuc cells expressing miniSINEUPs also displayed elevated levels of secreted nLuc, when compared to EV alone (FIG. 2d). Expression of miniSINEUPs in HEK-GDNF-nLuc was without effect on target GDNF mRNA expression (FIG. 2e).

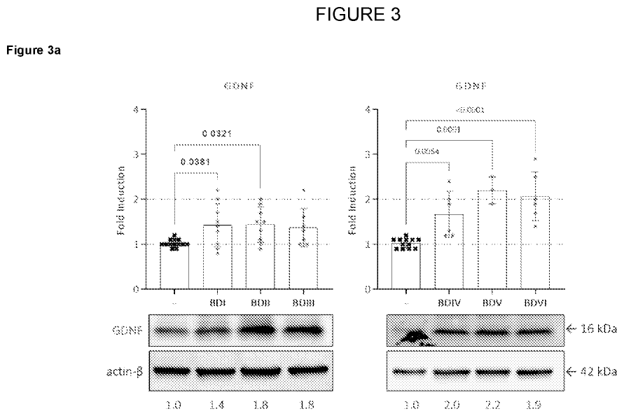

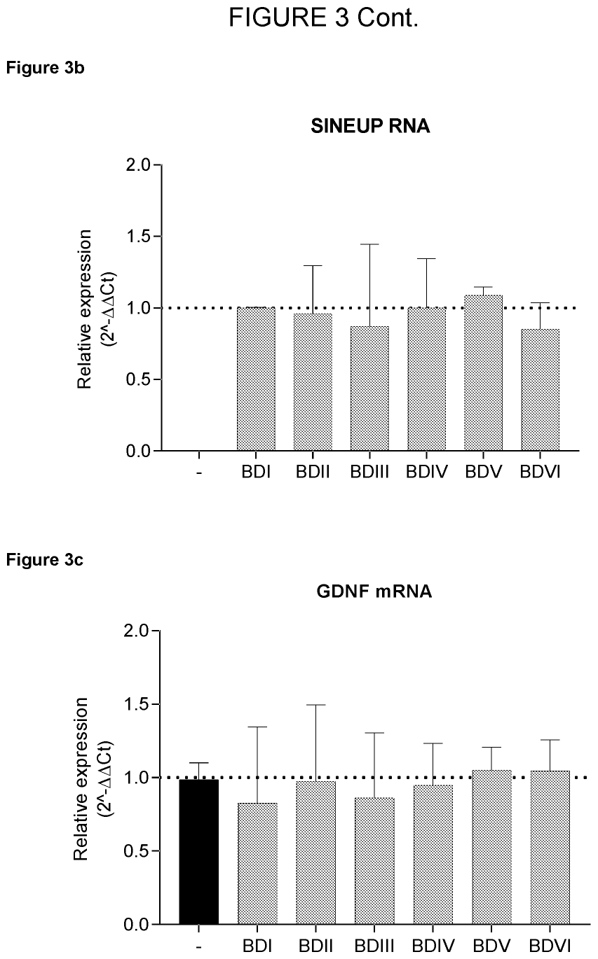

Further human GDNF targeting (FIG. 3): Hela cells were seeded into 6 well plates, 24 hrs before transfection. microSINEUPs to human GDNF (hGDNF) with the following BDs were tested: BDI: −14/−1 (methionine 1; M1); BDII: −14/+4 (methionine 1; M1); BDIII: −15/+4 (methionine 1; M1); BDIV: −14/+4 (methionine 2; M2); BDV: −25/−1 (methionine 2; M2); BDVI: −42/+4 (methionine 3; M3). Cells were transfected for 48 hrs with either microSINEUPs or empty vector using lipofectamine 2000. Cell lysates were harvested using standard methods (Espinoza et al., 2019; Bon et al., 2019) and samples were run on 4-20% mini-PROTEAN TGX precast protein gels. The primary antibody to human GDNF was Anti-GDNF antibody (Thermo Fisher Scientific, PA1-9524), diluted 1:1000 and detected using Goat anti-Chicken IgY (H+L) HRP-conjugated (Thermo Fisher Scientific, A16054) secondary antibody. Normalization to housekeeping protein B-actin was carried out by using an Anti-β-Actin-Peroxidase antibody (Sigma-Aldrich, A3854). Data were analysed using ordinary one-way anova; n≥3.

Preliminary data demonstrate an effect of microSINEUP-hGDNF on endogenous hGDNF protein amount in Hela cells. Representative examples are shown in FIG. 3. Upon 48 hours from transfection, microSINEUP-hGDNF caused increased levels of hGDNF protein, when compared to an empty vector (−) (FIG. 3a). microSINEUPs expression did not affect hGDNF mRNA levels, as expected for a post-transcriptional mechanism (FIGS. 3b and 3c).

### Example 4

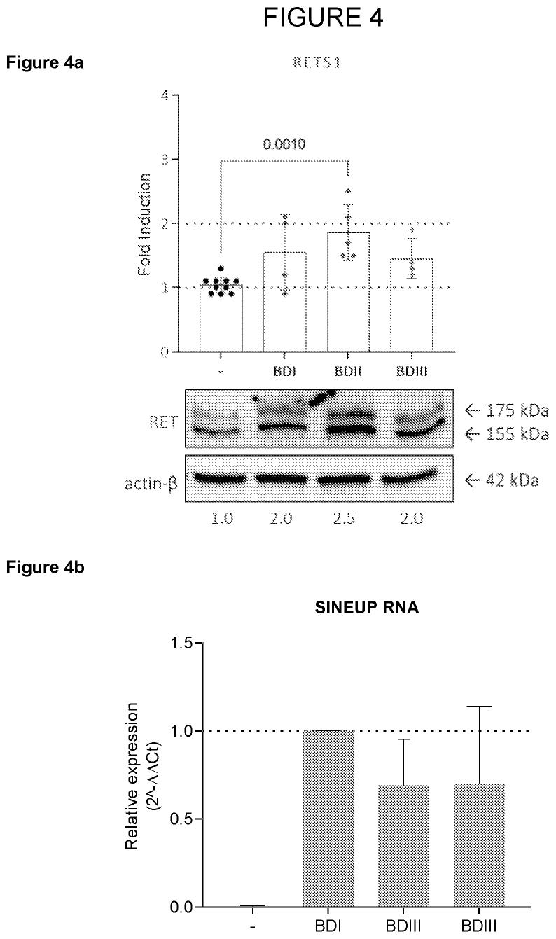

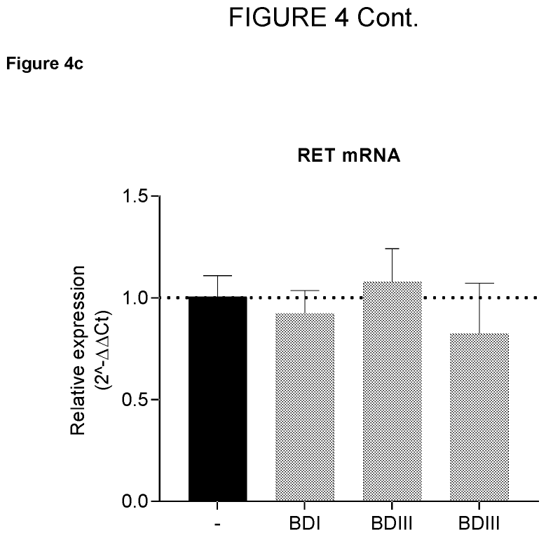

Human cRET targeting (FIG. 4): Hela cells were seeded into 24 well plates, 24 hrs before transfection. Functional nucleic acids to human cRET (hRET) with the following BDs were tested: BDI: −14/+4 (methionine 1; M1); BDII: −14/+4 (methionine 2; M2); BDIII: −14/+4 (methionine 3; M3). Cells were transfected for 48 hrs with either SINEUPs or empty vector using lipofectamine. Cell lysates were harvested using standard methods (Espinoza et al., 2019; Bon et al., 2019) and samples were run on 4-20% mini-PROTEAN TGX precast protein gels. The primary antibody to human cRET was anti-Ret antibody (Abcam; ab134100) and was used at 1:1000 and was detected using Goat anti-Rabbit IgG (Thermo Fisher Scientific; G-21234) secondary antibody. Normalization to housekeeping protein β-actin was enabled with detection of this protein using β-actin-HRP antibody (Anti-β-Actin-Peroxidase antibody; Sigma-Aldrich A3854). Data was analysed using ordinary one-way anova; n=>3.

Preliminary data demonstrating an effect of hcRET microSINEUPs on hcRET protein expression in Hela cells were generated. Exemplary data are shown in FIG. 4. Hela cells expressing microSINEUPs for 48 hrs displayed elevated levels of hcRET, when compared to empty vector alone (FIG. 4a). As determined by RT-qPCR, microSINEUPs to hcRET were expressed in the HeLa cell line (FIG. 4b). Discrimination between SINEUPs was observed with BDII significantly elevating cRET protein expression to 1.9 fold above vector control cRET levels, compared to BDI and BDIII cRET microSINEUPs. Expression of microSINEUPs in Hela cells was without effect on target hcRET mRNA expression (FIG. 4c).

### Example 5

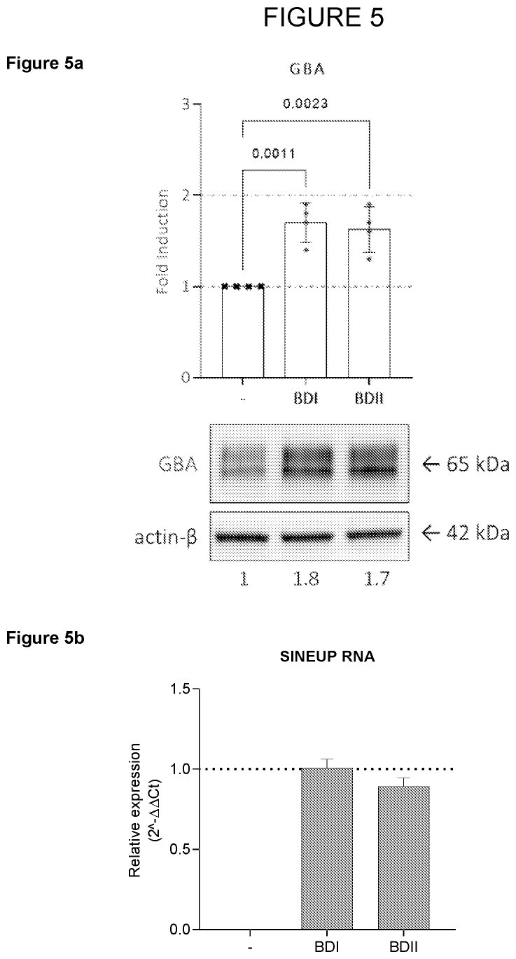

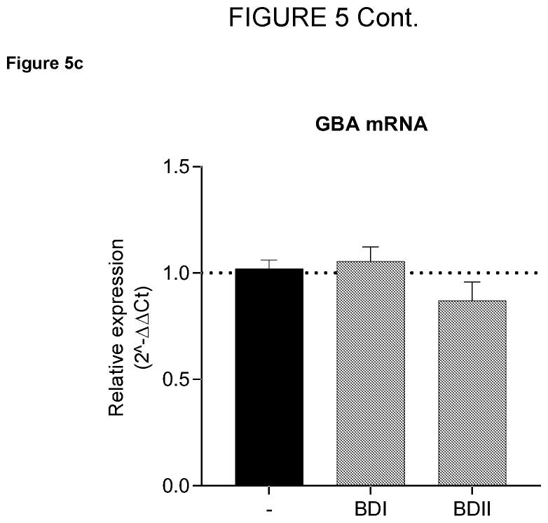

Human GBA targeting (FIG. 5): Hela cells were seeded into 6 well plates, 24 hrs before transfection. Functional nucleic acids to human GBA (hGBA) with the following BDs were tested: BDI: −20/−3 (methionine 1; M1); BDII: −14/+4 (methionine 1; M1). Cells were transfected for 48 hrs with either microSINEUPs or empty vector using lipofectamine 2000. Cell lysates were harvested using standard methods (Espinoza et al., 2019; Bon et al., 2019) and samples were run on 10% mini-PROTEAN TGX precast protein gels. The primary antibody to human GBA was Anti-GBA antibody (Thermo Fisher, OTI1D12), diluted 1:1000 and detected using Goat anti-Mouse IgG (H+L) HRP-conjugated (Thermo Fisher, 31430) secondary antibody. Normalization to housekeeping protein β-actin was carried out by using an Anti-β-Actin-Peroxidase antibody (Sigma-Aldrich, A3854). Data were analysed using ordinary one-way anova; n≥3.

Preliminary data demonstrating an effect of functional nucleic acids targeting hGBA on endogenous hGBA protein expression in Hela cells. Representative examples are shown in FIG. 5. Upon 48 hours from transfection, the functional nucleic acids (microSINEUPs) caused increased levels of hGBA protein, when compared to an empty vector (FIG. 5a). microSINEUPs expression did not affect hGBA mRNA levels, as expected for a post-transcriptional mechanisms (FIGS. 5b and c).

### Example 6

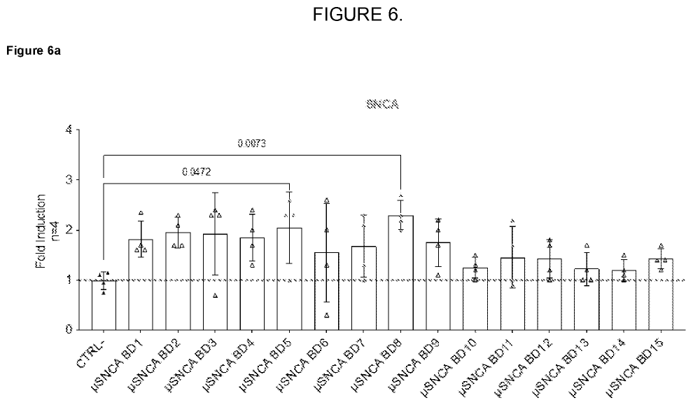

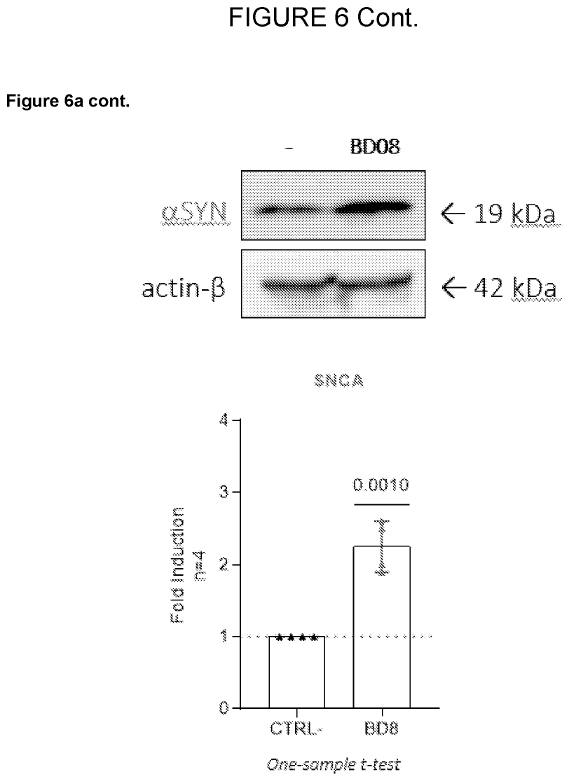

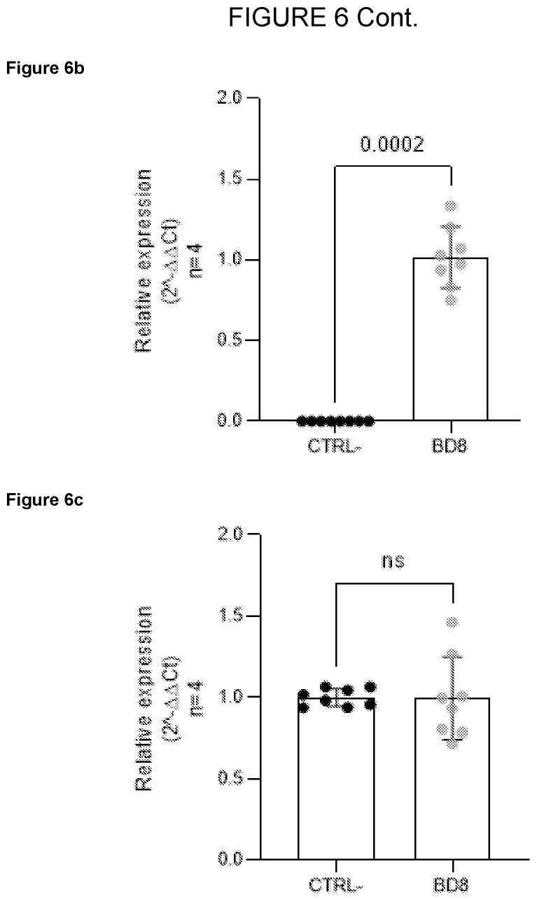

Human alpha synuclein (hαSYN) targeting (FIG. 6): Hela cells were seeded into 24 well plates, 24 hrs before transfection. MicroSINEUPs to human alpha synuclein (hαSYN or hSNCA) with various BDs were evaluated, as detailed in table 9. Cells were transfected for 48 hrs with either SINEUPs or empty vector using lipofectamine. Cell lysates were harvested using standard methods (Espinoza et al., 2019; Bon et al., 2019) and 10 μg/lane samples were run on 4-20% mini-PROTEAN TGX precast protein gels. The primary antibody to human αSYN was Abcam ab138501 and was used at 1:1000 overnight and was detected using anti-rabbit HRP secondary antibody. Normalization to housekeeping protein β-actin was enabled with detection of this protein using β-actin-HRP antibody (Sigma-Aldrich, A3854).

Preliminary data demonstrating an effect of hαSYN microSINEUPs were generated. Exemplary data are shown in FIG. 6. As determined by RT-qPCR, microSINEUP BD8 to hαSYN was expressed in the HeLa cell line (FIG. 6b). Hela cells expressing specific hαSYN microSINEUPs for 48 hrs displayed elevated levels of hαSYN, when compared to EV alone (FIG. 6a). Discrimination between SINEUPs was observed with e.g. BD8 significantly elevated hαSYN protein expression ˜2.3 fold above EV control hαSYN levels, compared to other microSINEUPs tested. Expression of microSINEUPs in Hela cells was without effect on target hαSYN mRNA expression (FIG. 6c).

TABLE 9
Summary of designed binding domains for hαSYN (hSNCA)

| | BD | Position relative |
|---|---|---|
| Target | Identifier | to M1 |
| hSNCA | BD01 | −34/−17 |
| hSNCA | BD02 | −40/−3 |
| hSNCA | BD03 | −40/−11 |
| hSNCA | BD04 | −20/−3 |
| hSNCA | BD05 | −40/−1 |
| hSNCA | BD06 | −26/−1 |
| hSNCA | BD07 | −14/−1 |
| hSNCA | BD08 | −14/+4 |
| hSNCA | BD09 | −15/+4 |
| hSNCA | BD10 | −16/+4 |
| hSNCA | BD11 | −40/+4 |
| hSNCA | BD12 | −32/+8 |
| hSNCA | BD13 | −54/+16 |
| hSNCA | BD14 | −25/+16 |
| hSNCA | BD15 | −20/+1 |

TABLE 10
Summary of binding domain nomenclature

| Name in | | | | SEQ |
|---|---|---|---|---|
| Examples/ | FIG./ | | Name in Table 1 | ID |
| Figures | Example | Target | or Table 7 | NO |
| GDNF BD-A | 2/3 | −14/+4 M1 | hGDNF BD6 M1 | 85 |
| GDNF BD-B | 2/3 | −40/+4 M1 | hGDNF BD10 M1 | 89 |
| GDNF BD-C | 2/3 | −56/+14 M1 | hGDNF BD12 M1 | 91 |
| GDNF BD-D | 2/3 | −14/+4 M1 | hGDNF BD6 M1 | 85 |
| GDNF BD-E | 2/3 | −40/+4 M1 | hGDNF BD10 M1 | 89 |
| GDNF BDI | 3/3 | −14/−1 M1 | hGDNF BD5 M1 | 84 |
| GDNF BDII | 3/3 | −14/+4 M1 | hGDNF BD6 M1 | 85 |
| GDNF BDIII | 3/3 | −15/+4 M1 | hGDNF BD7 M1 | 86 |
| GDNF BDIV | 3/3 | −14/+4 M2 | hGDNF BD15 M2 | 94 |
| GDNF BDV | 3/3 | −25/−1 M2 | hGDNF BD16 M2 | 95 |
| GDNF BDVI | 3/3 | −42/+4 M3 | hGDNF BD17 M3 | 96 |
| CRET BDI | 4/4 | −14/+4 M1 | hRET BD7 M1 | 112 |
| CRET BDII | 4/4 | −14/+4 M2 | hRET BD13 M2 | 118 |
| CRET BDIII | 4/4 | −14/+4 M3 | hRET DB15 M3 | 120 |
| GBA BDII | 5/5 | −14/+4 M1 | hGBA BD7 | 144 |
| SNCA BD8 | 6/6 | −14/+4 M1 | hSNCA BD8 | 187 |

SEQUENCE LISTING

| SEQ ID NO | Sequence | Name/Description |
|---|---|---|
| 1 | CAGUGCUAGAGGAGGUCAGAAGAGGGCAUUGGAUCCCCCAGAACUGGAGUUAUACGGU | AS Uchl1 SINEB2 |
| | AACCUCGUGGUGGUUGUGAACCACCAUGUGGAUGGAUAUUGAGUUCCAAACACUGGUC | |
| | CUGUGCAAGAGCAUCCAGUGCUCUUAAGUGCUGAGCCAUCUCUUUAGCUCC | |
| 2 | GAACUGGAGUUAUACGGUAACCUCGUGGUGGUUGUGAACCACCAUGUGGAUGGAUAUU | nt 44-120 of inverted SINE B2 |
| | GAGUUCCAAACACUGGUCC | transposable element derived from AS |
| | | Uchl1 |
| 3 | CCUCGUGGUGGUUGUGAACCACCAUGUGG | nt 64-92 of inverted SINE B2 transposable |
| | | element derived from AS Uchl1 |
| 4 | GUUAUACGGUAACCUCGUGGUGGUUGUGAACCACCAUGUGGAUGGAUAUUGAGUUCCA | nt 52-112 of inverted SINE B2 |
| | AAC | transposable element derived from AS |
| | | Uchl1 |
| 5 | AUCCCCCAGAACUGGAGUUAUACGGUAACCUCGUGGUGGUUGUGAACCACCAUGUGGA | nt 36-133 of inverted SINE B2 |
| | UGGAUAUUGAGUUCCAAACACUGGUCCUGUGCAAGAGCAU | transposable element derived from AS |
| | | Uchl1 |
| 6 | GAAGAGGGCA UUGGAUCCCC CAGAACUGGA GUUAUACGGU AACCUCGUGG | nt 22-150 of inverted SINE B2 |
| | UGGUUGUGAA CCACCAUGUG GAUGGAUAUU GAGUUCCAAA CACUGGUCCU | transposable element derived from AS |
| | GUGCAAGAGC AUCCAGUGCU CUUAAGUGC | Uchl1 |
| 7 | GGGCAGUGCU AGAGGAGGUC AGAAGAGGGC AUUGGAUCCC CCAGAACUGG | nt 1-183 of inverted SINE B2 transposable |
| | AGUUAUACGG UAACCUCGUG GUGGUUGUGA ACCACCAUGU GGAUGGAUAU | element derived from AS Uchl1 |
| | UGAGUUCCAA ACACUGGUCC UGUGCAAGAG CAUCCAGUGC UCUUAAGUGC | |
| | UGAGCCAUCU CUUUAGCUCC AGUCUCUUAA GCU | |
| 8 | GAACUGGAGU UAUACGGUAA CCUCGUGGUG GUUGUGAACC ACCAUGUGGA | nt 44-110 of inverted SINE B2 |
| | UGGAUAUUGA GUUCCAA | transposable element derived from AS |
| | | Uchl1 |
| 9 | GGAUCCCCCA GAACUGGAGU UAUACGGUAA CCUCGUGGUG GUUGUGAACC | nt 34-140 of inverted SINE B2 |
| | ACCAUGUGGA UGGAUAUUGA GUUCCAAACA CUGGUCCUGU GCAAGAGCAU | transposable element derived from AS |
| | CCAGUGC | Uchl1 |
| 10 | AGAGGGCAUU GGAUCCCCCA GAACUGGAGU UAUACGGUAA CCUCGUGGUG | nt 24-150 of inverted SINE B2 |
| | GUUGUGAACC ACCAUGUGGA UGGAUAUUGA GUUCCAAACA CUGGUCCUGU | transposable element derived from AS |
| | GCAAGAGCAU CCAGUGCUCU UAAGUGC | Uchl1 |
| 11 | GGUAACCUCG UGGUGGUUGU GAACCACCAU GUGGAUGG | nt 59-96 of inverted SINE B2 transposable |
| | | element derived from AS Uchl1 |
| 12 | CAGUGCUAGA GGAGGUCAGA AGAGGGCAUU GGAUCCCCCA GAACUGGAGU | G60A mutant of inverted SINE B2 |
| | UAUACGAUAA CCUCGUGGUG GUUGUGAACC ACCAUGUGGA UGGAUAUUGA | transposable element derived from AS |
| | GUUCCAAACA CUGGUCCUGU GCAAGAGCAU CCAGUGCUCU UAAGUGCUGA | Uchl1 |
| | GCCAUCUCUU UAGCUCCAGU CUCUUAAGCU | |
| 13 | CAGUGCUAGA GGAGGUCAGA AGAGGGCAUU GGAUCCCCCA GAACUGGAGU | G60C mutant of inverted SINE B2 |
| | UAUACGCUAA CCUCGUGGUG GUUGUGAACC ACCAUGUGGA UGGAUAUUGA | transposable element derived from AS |
| | GUUCCAAACA CUGGUCCUGU GCAAGAGCAU CCAGUGCUCU UAAGUGCUGA | Uchl1 |
| | GCCAUCUCUU UAGCUCCAGU CUCUUAAGCU | |
| 14 | CAGUGCUAGA GGAGGUCAGA AGAGGGCAUU GGAUCCCCCA GAACUGGCGU | A51C mutant of inverted SINE B2 |
| | UAUACGGUAA CCUCGUGGUG GUUGUGAACC ACCAUGUGGA UGGAUAUUGA | transposable element derived from AS |
| | GUUCCAAACA CUGGUCCUGU GCAAGAGCAU CCAGUGCUCU UAAGUGCUGA | Uchl1 |
| | GCCAUCUCUU UAGCUCCAGU CUCUUAAGCU | |
| 15 | CAGUGCUAGA GGAGGUCAGA AGAGGGCAUU GGAUCCCCCA GAAGUGGAGU | C47G and G117C mutant of inverted |
| | UAUACGGUAA CCUCGUGGUG GUUGUGAACC ACCAUGUGGA UGGAUAUUGA | SINE B2 transposable element derived |
| | GUUCCAAACA CUGCUCCUGU GCAAGAGCAU CCAGUGCUCU UAAGUGCUGA | from AS Uchl1 |
| | GCCAUCUCUU UAGCUCCAGU CUCUUAAGCU | |
| 16 | CAGUGCUAGA GGAGGUCAGA AGAGGGCAUU GGAUCCCCCA GAUGGUGAGU | 46-49/115-118 stem swap mutant of |
| | UAUACGGUAA CCUCGUGGUG GUUGUGAACC ACCAUGUGGA UGGAUAUUGA | inverted SINE B2 transposable element |
| | GUUCCAAACA CGUCACCUGU GCAAGAGCAU CCAGUGCUCU UAAGUGCUGA | derived from AS Uchl1 |
| | GCCAUCUCUU UAGCUCCAGU CUCUUAAGCU | |
| 17 | CAGUGCUAGA GGAGGUCAGA AGAGGGCAUU GGAUCCCCCA GAACUGCACU | 50-53 and 112-114 base swap mutant of |
| | AUACGGUAAC CUCGUGGUGG UUGUGAACCA CCAUGUGGAU GGAUAUUGAG | inverted SINE B2 transposable element |
| | UUCCAAAUGA GUGGUCCUGU GCAAGAGCAU CCAGUGCUCU UAAGUGCUGA | derived from AS Uchl1 |
| | GCCAUCUCUU UAGCUCCAGU CUCUUAAGCU | |
| 18 | GGGCAUUGGA UCCCCCAGAA CUGGAGUUAU ACGGUAACCU CGUGGUGGUU | nt 27-142 of inverted SINE B2 |
| | GUGAACCACC AUGUGGAUGG AUAUUGAGUU CCAAACACUG GUCCUGUGCA | transposable element derived from AS |
| | AGAGCAUCCA GUGCUC | Uchl1 |
| 19 | GGACUGGAGU UAUACGGUAA CCUCGUGGUG GUUGUGAACC ACCAUGUGGA | nt 44-120 A45G mutant of inverted SINE |
| | UGGAUAUUGA GUUCCAAACA CUGGUCC | B2 transposable element derived from AS |
| | | Uchl1 |
| 20 | GAACUGGCGU UAUACGGUAA CCUCGUGGUG GUUGUGAACC ACCAUGUGGA | nt 44-120 A51C mutant of inverted sine |
| | UGGAUAUUGA GUUCCAAACA CUGGUCC | B2 transposable element derived from AS |
| | | Uchl1 |
| 21 | GAUGCCUUAG AAGUGGAGUU AAGAGUUGUG AGCUGCCGUU UUUUGGUUCU | AS Txnip SINEB2 |
| | GGGACUCGAA CUCGUUUCCU CUGAUACUAU CAACCACCAA GCCAUCUCUU | |
| | CAGCCCC | |
| 22 | GCCAGAAGAA GUUGUGGGAU UCCCUGGAAC UGGAGCAACC AACAGUUUGU | AS Uxt SINEB2 a |
| | GUGCACCAUG UGGGUAAUGG GAAUCGAACC UGGGUCCUCU AUAAGACUGG | |
| | CCAGUGCUCU UAACUACUGA GGUGCAUUUC U | |
| 23 | UUAUUUUAAA UAUAUGAGUA UUUCACCUGC AUAGGCGCAC AGUACCCACA | AS Uxt SINEB2 b |
| | GAGACUAGAA GAGGGUGGCA GAUCUCCUGA GACUGGAGUU AAUGCUUGUG | |
| | AGCUGCCAUG UGGAUGCUGG AAAUCAAACC CAGGUCCUUU GGAAGGCAGG | |
| | CAGGUGCUCU UAAUCAUGGA AGCAUCUCUU CAGCUCC | |
| 24 | CAGCGACAUC AGAAGAGGAU AUUGGAUCCC AUUACAGAUG GUUGAAGGCC | AS Gadd45alpha SINEB2 a |
| | ACCAUGUCGU UGCUGGGAAU GAACUCAAGA CCUCUGGAAG AGCAGUCAGU | |
| | GCUCUUAACC UCUGAGCCAU CUCUCCAGCC C | |
| 25 | AUCCCCUCCA AAGCUCAAGA UGGUUGUAAG CCACCCUGUG AUUGCUGGGA | AS Gadd45alpha SINEB2 b |
| | UUUGAACUCA AGACCUCCGG AAGAGCAAUU AGUGCUCUUA ACCGCUGAGC | |
| | AAUCUCUCCA GCCC | |
| 26 | GUGCAGUGCU AGAGGAGGUC AGAAGAGGGC AUUGGAUCCC CCAGAACUGG | AS Uchl1 SINEB2 + AS Uxt SINEB2 b |
| | AGUUAUACGG UAACCUCGUG GUGGUUGUGA ACCACCAUGU GGAUGGAUAU | |
| | UGAGUUCCAA ACACUGGUCC UGUGCAAGAG CAUCCAGUGC UCUUAAGUGC | |
| | UGAGCCAUCU CUUUAGCUCC UUAUUUUAAA UAUAUGAGUA UUUCACCUGC | |
| | AUAGGCGCAC AGUACCCACA GAGACUAGAA GAGGGUGGCA GAUCUCCUGA | |
| | GACUGGAGUU AAUGCUUGUG AGCUGCCAUG UGGAUGCUGG AAAUCAAACC | |
| | CAGGUCCUUU GGAAGGCAGG CAGGUGCUCU UAAUCAUGGA AGCAUCUCUU | |
| | CAGCUCC | |
| 27 | GUGCAGUGCU AGAGGAGGUC AGAAGAGGGC AUUGGAUCCC CCAGAACUGG | 3x AS Uchl1 SINEB2 |
| | AGUUAUACGG UAACCUCGUG GUGGUUGUGA ACCACCAUGU GGAUGGAUAU | |
| | UGAGUUCCAA ACACUGGUCC UGUGCAAGAG CAUCCAGUGC UCUUAAGUGC | |
| | UGAGCCAUCU CUUUAGCUCC GUGCGAAUUC GGUGCAGUGC UAGAGGAGGU | |
| | CAGAAGAGGG CAUUGGAUCC CCCAGAACUG GAGUUAUACG GUAACCUCGU | |
| | GGUGGUUGUG AACCACCAUG UGGAUGGAUA UUGAGUUCCA AACACUGGUC | |
| | CUGUGCAAGA GCAUCCAGUG CUCUUAAGUG CUGAGCCAUC UCUUUAGCUC | |
| | CGUGCGAAUU CGGUGCAGUG CUAGAGGAGG UCAGAAGAGG GCAUUGGAUC | |
| | CCCCAGAACU GGAGUUAUAC GGUAACCUCG UGGUGGUUGU GAACCACCAU | |
| | GUGGAUGGAU AUUGAGUUCC AAACACUGGU CCUGUGCAAG AGCAUCCAGU | |
| | GCUCUUAAGU GCUGAGCCAU CUCUUUAGCU CC | |
| 28 | UUUUUUUAAA AAUUUAUUUU UAUUUUAUGU GUAUGAGUGU UUUGCCUGCA | SINEB2/B3 consensus sequence from |
| | UGUAUGUCUG UGUACCACGU GCGUGCCUGG UGCCCGCGGA GGCCAGAAGA | RepBase |
| | GGGCGUCGGA UCCCCUGGAA CUGGAGUUAC AGAUGGUUGU GAGCCGCCAU | |
| | GUGGGUGCUG GGAAUCGAAC CCGGGUCCUC UGGAAGAGCA GCCAGUGCUC | |
| | UUAACCGCUG AGCCAUCUCU CCAGCCCC | |
| 29 | UUUUUUUUAC UUGUAUAGGU GUUUUGCCUG CAUGUGUAUC UAUCUAUGUA | AS Nars2 SINEB2/B3 |
| | CCGAAUAUGU UCCUGGUAUC CACAGAGACC AAAAGUGGAU GUUGUAUCUC | |
| | CUGAAAUUGG AGUCAUAGAC AGUUAUGAGC UGCCAUUUGA GUGCUUGGAA | |
| | UAGAACCCAG GUCCUCUUAA AGAGCAUCCA GUGCUCUUAA AAACUGAGAC | |
| | AUCUCUGUAG CCUC | |
| 30 | UUUAUUUUGC UUUAUGUGUC UGAGUGUUUG CUUGAAUGUA UGUCUGUGUA | AS Abhd11 SINEB2/B3 |
| | CCACGCCUGU ACCUUGUGCC UUCAGAGUUG AGAGGAGGGC AUAGGAUCUC | |
| | CUGGAACUGG AAUUGCAGGU GGUUGUGAGC CACCCUGUGG GUCCUGGGGA | |
| | CCAUACUCCA GCAAGAACAU CAUGUGCUCU UAAUUCCUGA GUCUCCAACC | |
| 31 | UUUAUUUACU UAUCUUUAUG UGUAUGAGUG UGUUGUCAGA CUGUUAUGUC | AS Ebp4.9 SINEB2/B3 |
| | UGUGUGUCAC AUGCAUGCCU GCUGUUCAUG GAGUCCAGAA GAGGGCAUCG | |
| | GAUCCCCUGG AACUGGAGUU ACAGAUGAGU GGCCAUGUGA AUGUUAAGAA | |
| | CCAAACCUGG GUCCUCUGAA AGAGCAGACA AUGCUCUUAA CUACUGAGCU | |
| | GUCUCUCCAG CCCC | |
| 32 | UUAUUUUAUU CGUGUAAGUG UUUUGCCAGC AUCUAUGUCU UCGCACUAUG | AS Wfdc5 SINEB2/B3 |
| | UGCAGGUCUG GUGCCUGAGG GGUCCAGACG AGAGCACUGG GUCUCCGGGA | |
| | ACUGGAGUUA CAGAUCAUUG UGAGCCACCA UGUGGGUGCA GGGAAUCGAA | |
| | CCUGGGACCU CUGGAGGAGC AGCCACUGCU CUUAACCACU ACACUAUUUC UCCAG | |
| 33 | UCUGUGGACC ACUGUGUACA GAAGCCUGAG AAGGCUAGCA GAUCCCCAGA | AS Pgbd1 SINEB2/B3 |
| | ACUGGAACUG UGAGACGCUG UGCUAUGGAG GUGCUAGGAA CUGAAAAUGG | |
| | AUGGGUCCUC UGCAAGAGCA G | |
| 34 | UUGUUUUAAU UGAAUGGCUA UAGGGUGUUU CUUCUGUAUG UAUAUCUAUG | AS Gsk3b SINEB2/B3 |
| | UUUGGUACCU ACAGAGGCAU CAGAUCCUCU GGAACUGUAG UUGCUGACAG | |
| | UUGUGAGCUG UCAUGGGGAU GCUGGAAUUG AACCUGGAUC CUAUGAAAGA | |
| | ACAGCCAGUG UUCUUAACCG CUGAGCUAUC UCUCCAGGCC C | |
| 35 | UUUUUUUUUU AAUUUUAAAA AAAAAGAUUU UAUUUAUUUA UUUUAUAUAU | AS Rhod SINEB2_Mm2 |
| | GAUGAGUACA CUGUCACUCU UUUCAGACAC CCUAGAAAAG GGGGGCAUCA | |
| | GAUCCCAUUA CAGAUGGUUG UGAGCCACAU GGUUGCUGGG AAUUGACCUC | |
| | AGGACCUCUG AAAGAGCAGU CAGUGCUCUC AACCUUUGAG UCAUCUCUCC AGCCC | |
| 36 | AUGUAUAUCU GUAAUGGGAC AUACUCACAU ACAUGGGCAC GUGAGUAUAA | AS Rhod SINEB2/B3A |
| | AAGGCCAGAA GAGAGCACUG GACCCUCUGG AGUUGAGAUU CUAAGCAGUU | |
| | GUGAACCAUC UGAUGUAGGU GCUGGGAACU GAACUUGGGU CCUUUGCUAG | |
| | AGAAGUAUGU CUCUUAACCA CUGAGCCGUA UCUCCAUCCC | |
| 37 | UAAAGAUUUA UUCAUUAAGU ACACUGUAGC UAUCUUCAGA CGCAUCAGAA | AS E4f1 SINEB2_Mm2 |
| | GAGGGCGUCA GAUCUCUUUA CAGGUGGUUG UGAGCCACCA UGUGGUUGCU | |
| | GGAAUUUGAA CUCAGGACCU UCAAAAGAGC AGUCAGUGUU CUUAACCGCU | |
| | GAGCCAUCUC UCCAACCCC | |
| 38 | UUAUUUAUUA UAAGUACACU GUAGCUGUCU UCAGACACAA CAAAAGAGGG | AS E4f1 SINEB2_Mm1t |
| | CGUCAGAUCU CAUUACAGGU GGUUGAGCCA CCAUGUGGUU GCUGGGAUUU | |
| | GAACUCAGGA CCUUCAGAAC AGUCAGUGCU CUUACCCACU GAGCCAGCGA | |
| | GCCAGCCCC | |
| 39 | GAACUGGAGU UAUACGGUAA CCUCGUGGUG GUUGGGAACC ACCAUGUGGA | 44-120 U78G SINEUP |
| | UGGAUAUUGA GUUCCAAACA CUGGUCC | |
| 40 | GAACUGGAGU UAUACGGUAA CCUCGUGGUG GUUCCCAACC ACCAUGUGGA | 44-120 GUG77-79CCC SINEUP |
| | UGGAUAUUGA GUUCCAAACA CUGGUCC | |
| 41 | GGACCGGAGU UAUACGGUAA CCGCGUGGUG GUUGUGAACC ACCACGCGGA | 44-120 SINEUP strong |
| | UGGAUAUUGA GUUCCAAACA CCGGUCC | |
| 42 | GAACUAGAGU UAUACGGUAA CCACAUGGUG GUUGUGAACC ACCAUGUGGA | 44-120 SINEUP weak |
| | UGGAUAUUGA GUUCCAAACA CUAGUUC | |
| 43 | UUAUUUUAAA UAUAUGAGUA UUUCACCUGC AUAGGCGCAC AGUACCCACA | SINEUP 071 and miniSINEUP 071 |
| | GAGACUAGAA GAGGGUAGUA GAUCCCCUAG AACUGGAGUU AUACGGUAAC | effector domain |
| | CUCGUGGUGG UUGUGAGCUA CCAUGUGGAU GGAUACUGGG AAUCAAACCC | |
| | AGGUCCUGUG GAAGGCAGGC AGGUGCUCUC AAGCACUGAG CCAUCUCUUC AGCUCC | |
| 44 | UUAUUUUAAA UAUAUGAGUA UUUCACCUGC AUAGGCGCAC AGUGCUCAAG | SINEUP 072 and miniSINEUP 072 |
| | GAGAUCAGAA GAGGGCAUCA GAUCUCCUGA GACUGGAGUU AUACGGUAAC | effector domain |
| | CUCGUGAUGG UUGUGAACUA CCAUGUGGAU GGAUAUUGAG UUCCAAACAC | |
| | AGGUCCUGUG CAAGAGCAGC AGGUGCUCUU AAGCACGGAA CCAUCUCUUU AGCUCC | |
| 45 | GAGGCUAGAA GAGGGUAUCA GAUCCCCUGA GACUGGAGUU AUACGGUAAC | SINEUP 073 and miniSINEUP 073 |
| | CUCGUGGUGG UUGUGAGCCA CCAUGUGGAU GGAUACUGAG AACCAAACCC | effector domain |
| | UGGUCCUGUG CAAGAGCAUC AGGUGCUCUU AAGCACGGAA CCAUCUCUUC AGCUCC | |
| 46 | GUCCUGUGCA AGAGCAUCGA ACUCGGUGCU CUUAAGCACA GAAGCCACCA | SINEUP 074 and miniSINEUP 074 |
| | AGCCAUCUCU UCAGCCCC | effector domain |
| 47 | CAGUGCUAGA GGAGGUCAGA AGAGGGCAUC CCCCAGCCUC GUGGUGGUUG | SINEUP 075 and miniSINEUP 075 |
| | UGAACCACCA UGUGGCUGUG CAAGAGCAUG CUCUUAAGUG CUGAGCCAUC | effector domain |
| | UCUUUAGCUC | |
| 48 | GAGGGCAUUG GAUCCCCCAG AACUGGAGUU AUACGGUAAC CUCGUGGUGG | 24-150 effector domain |
| | UUGUGAACCA CCAUGUGGAU GGAUAUUGAG UUCCAAACAC UGGUCCUGUG | |
| | CAAGAGCAUC CAGUGCUCUU AAGUGC | |
| 49 | GGAUCCCCCA GAACUGGAGU UAUACGGUAA CCUCGUGGUG GUUGUGAACC | C_34-122 effector domain |
| | ACCAUGUGGA UGGAUAUUGA GUUCCAAACA CUGGUCCUG | |
| 50 | UGCUAGAGGA GGUCAGAAGA GGGCAUUGGA UGCAAAUCCA GUGCUCUUAA | TM effector domain |
| | GUGCUGAGCC AUCUCUUUAG CU | |
| 51 | GAGGGCAUUG GAUCCCCCAG AACUGGAGUU AUACGGUAAC GAUGGAUAUU | MC2 effector domain |
| | GAGUUCCAAA CACUGGUCCU GUGCAAGAGC AUCCAGUGCU CUUA | |
| 52 | GCCAGCCCCC UGAUGGGGGC GACACUCCAC CAUGAAUCAC UCCCCUGUGA | HCV(d) IRES |
| | GGAACUACUG UCUUCACGCA GAAAGCGUCU AGCCAUGGCG UUAGUAUGAG | |
| | UGUCGUGCAG CCUCCAGGAC CCCCCCUCCC GGGAGAGCCA UAGUGGUCUG | |
| | CGGAACCGGU GAGUACACCG GAAUUGCCAG GACGACCGGG UCCUUUCUUG | |
| | GAUAAACCCG CUCAAUGCCU GGAGAUUUGG GCGUGCCCCC GCAAGACUGC | |
| | UAGCCGAGUA GUGUUGGGUC GCGAAAGGCC UUGUGGUACU GCCUGAUAGG | |
| | GUGCUUGCGA GUGCCCCGGG AGGUCUCGUA GACCGUGCAC CAUGAGCACG | |
| | AAUCCUAAAC CUCAAAGAAA AACCAAACGU AAC | |
| 53 | GUUACGUUUG GUUUUUCUUU GAGGUUUAGG AUUCGUGCUC AUGGUGCACG | HCV(i) IRES |
| | GUCUACGAGA CCUCCCGGGG CACUCGCAAG CACCCUAUCA GGCAGUACCA | |
| | CAAGGCCUUU CGCGACCCAA CACUACUCGG CUAGCAGUCU UGCGGGGGCA | |
| | CGCCCAAAUC UCCAGGCAUU GAGCGGGUUU AUCCAAGAAA GGACCCGGUC | |
| | GUCCUGGCAA UUCCGGUGUA CUCACCGGUU CCGCAGACCA CUAUGGCUCU | |
| | CCCGGGAGGG GGGGUCCUGG AGGCUGCACG ACACUCAUAC UAACGCCAUG | |
| | GCUAGACGCU UUCUGCGUGA AGACAGUAGU UCCUCACAGG GGAGUGAUUC | |
| | AUGGUGGAGU GUCGCCCCCA UCAGGGGGCU GGC | |
| 54 | AUGAGUCUGG ACAUCCCUCA CCGGUGACGG UGGUCCAGGC UGCGUUGGCG | Polio(d) IRES |
| | GCCUACCUAU GGCUAACGCC AUGGGACGCU AGUUGUGAAC AAGGUGUGAA | |
| | GAGCCUAUUG AGCUACAUAA GAAUCCUCCG GCCCCUGAAU GCGGCUAAUC | |
| | CCAACCUCGG AGCAGGUGGU CACAAACCAG UGAUUGGCCU GUCGUAACGC | |
| | GCAAGUCCGU GGCGGAACCG ACUACUUUGG GUGUCCGUGU UUCCUUUUAU | |
| | UUUAUUGUGG CUGCUUAUGG UGACAAUCAC AGAUUGUUAU CAUAAAGCGA | |
| | AUUGGAUUGG CC | |
| 55 | GGCCAAUCCA AUUCGCUUUA UGAUAACAAU CUGUGAUUGU CACCAUAAGC | Polio(i) IRES |
| | AGCCACAAUA AAAUAAAAGG AAACACGGAC ACCCAAAGUA GUCGGUUCCG | |
| | CCACGGACUU GCGCGUUACG ACAGGCCAAU CACUGGUUUG UGACCACCUG | |
| | CUCCGAGGUU GGGAUUAGCC GCAUUCAGGG GCCGGAGGAU UCUUAUGUAG | |
| | CUCAAUAGGC UCUUCACACC UUGUUCACAA CUAGCGUCCC AUGGCGUUAG | |
| | CCAUAGGUAG GCCGCCAACG CAGCCUGGAC CACCGUCACC GGUGAGGGAU | |
| | GUCCAGACUC AU | |
| 56 | CCCCCCCUCU CCCUCCCCCC CCCCUAACGU UACUGGCCGA AGCCGCUUGG | EMCV(d) IRES |
| | AAUAAGGCCG GUGUGCGUUU GUCUAUAUGU UAUUUUCCAC CAUAUUGCCG | |
| | UCUUUUGGCA AUGUGAGGGC CCGGAAACCU GGCCCUGUCU UCUUGACGAG | |
| | CAUUCCUAGG GGUCUUUCCC CUCUCGCCAA AGGAAUGCAA GGUCUGUUGA | |
| | AUGUCGUGAA GGAAGCAGUU CCUCUGGAAG CUUCUUGAAG ACAAACAACG | |
| | UCUGUAGCGA CCCUUUGCAG GCAGCGGAAC CCCCCACCUG GCGACAGGUG | |
| | CCUCUGCGGC CAAAAGCCAC GUGUAUAAGA UACACCUGCA AAGGCGGCAC | |
| | AACCCCAGUG CCACGUUGUG AGUUGGAUAG UUGUGGAAAG AGUCAAAUGG | |
| | CUCUCCUCAA GCGUAUUCAA CAAGGGGCUG AAGGAUGCCC AGAAGGUACC | |
| | CCAUUGUAUG GGAUCUGAUC UGGGGCCUCG GUGCACAUGC UUUACAUGUG | |
| | UUUAGUCGAG GUUAAAAAAC GUCUAGGCCC CCCGAACCAC GGGGACGUGG | |
| | UUUUCCUUUG AAAAACACGA UGAUAA | |
| 57 | UUAUCAUCGU GUUUUUCAAA GGAAAACCAC GUCCCCGUGG UUCGGGGGGC | ECMV(i) IRES |
| | CUAGACGUUU UUUAACCUCG ACUAAACACA UGUAAAGCAU GUGCACCGAG | |
| | GCCCCAGAUC AGAUCCCAUA CAAUGGGGUA CCUUCUGGGC AUCCUUCAGC | |
| | CCCUUGUUGA AUACGCUUGA GGAGAGCCAU UUGACUCUUU CCACAACUAU | |
| | CCAACUCACA ACGUGGCACU GGGGUUGUGC CGCCUUUGCA GGUGUAUCUU | |
| | AUACACGUGG CUUUUGGCCG CAGAGGCACC UGUCGCCAGG UGGGGGGUUC | |
| | CGCUGCCUGC AAAGGGUCGC UACAGACGUU GUUUGUCUUC AAGAAGCUUC | |
| | CAGAGGAACU GCUUCCUUCA CGACAUUCAA CAGACCUUGC AUUCCUUUGG | |
| | CGAGAGGGGA AAGACCCCUA GGAAUGCUCG UCAAGAAGAC AGGGCCAGGU | |
| | UUCCGGGCCC UCACAUUGCC AAAAGACGGC AAUAUGGUGG AAAAUAACAU | |
| | AUAGACAAAC GCACACCGGC CUUAUUCCAA GCGGCUUCGG CCAGUAACGU | |
| | UAGGGGGGGG GGAGGGAGAG GGGGGG | |
| 58 | AAAGCAAAAA UGUGAUCUUG CUUGUAAAUA CAAUUUUGAG AGGUUAAUAA | CrPV(d) IRES |
| | AUUACAAGUA GUGCUAUUUU UGUAUUUAGG UUAGCUAUUU AGCUUUACGU | |
| | UCCAGGAUGC CUAGUGGCAG CCCCACAAUA UCCAGGAAGC CCUCUCUGCG | |
| | GUUUUUCAGA UUAGGUAGUC GAAAAACCUA AGAAAUUUAC CU | |
| 59 | AGGUAAAUUU CUUAGGUUUU UCGACUACCU AAUCUGAAAA ACCGCAGAGA | CrPV(i) IRES |
| | GGGCUUCCUG GAUAUUGUGG GGCUGCCACU AGGCAUCCUG GAACGUAAAG | |
| | CUAAAUAGCU AACCUAAAUA CAAAAAUAGC ACUACUUGUA AUUUAUUAAC | |
| | CUCUCAAAAU UGUAUUUACA AGCAAGAUCA CAUUUUUGCU UU | |
| 60 | CAGAGAUCCA GGGGAGGCGC CUGUGAGGCC CGGACCUGCC CCGGGGCGAA | Apaf-1(d) IRES |
| | GGGUAUGUGG CGAGACAGAG CCCUGCACCC CUAAUUCCCG GUGGAAAACU | |
| | CCUGUUGCCG UUUCCCUCCA CCGGCCUGGA GUCUCCCAGU CUUGUCCCGG | |
| | CAGUGCCGCC CUCCCCACUA AGACCUAGGC GCAAAGGCUU GGCUCAUGGU | |
| | UGACAGCUCA GAGAGAGAAA GAUCUGAGGG A | |
| 61 | UCCCUCAGAU CUUUCUCUCU CUGAGCUGUC AACCAUGAGC CAAGCCUUUG | Apaf-1(i) IRES |
| | CGCCUAGGUC UUAGUGGGGA GGGCGGCACU GCCGGGACAA GACUGGGAGA | |
| | CUCCAGGCCG GUGGAGGGAA ACGGCAACAG GAGUUUUCCA CCGGGAAUUA | |
| | GGGGUGCAGG GCUCUGUCUC GCCACAUACC CUUCGCCCCG GGGCAGGUCC | |
| | GGGCCUCACA GGCGCCUCCC CUGGAUCUCU G | |
| 62 | ACUUUUGGUG GGCAUUUAAA AAUGUGUGUG UAUGUGUAUA UAUGUAUGUG | ELG-1(d) IRES |
| | UAUGUAUGUG UAUAUAUGUA UAUGUAUGUA UGUAUCGCGU GUAUGUGUGU | |
| | AUGUAUGCAU GUGUAUGUAU GUAUAUGCAU GUAUGUGUAU GUGUAUAUAU | |
| | GUAUGUGUGU GUAUGUAUAU GUGUGUGUAU GUGUAUGUGU GUGUGUAUGU | |
| | GUGUGUGUAU GUAUGUAUGU AUGUAUAUGU AUUAUACACA UAUACACAUA | |
| | UUGGUUUUUU UAAUCAUUUG AGAGUUAGUU GAAGAUAAAA ACCCAUCACC | |
| | CCUAAAUGUA UUCCAAAGAA UAAGAACAUU GUUUUAUACA UAGCACACUU | |
| | AACAAAAUCA AGAAAUUUAA CAUUAAUACA GUACUGUUAC CUAAUCCGUA | |
| | GUCGAUUUUC AAAUUUUGUC AGUUGUUCCA AUAAUGUCCU UUAUAUAUUC | |
| | CCCGCCCAGC | |
| 63 | GCUGGGCGGG GAAUAUAUAA AGGACAUUAU UGGAACAACU GACAAAAUUU | ELG-1(i) IRES |
| | GAAAAUCGAC UACGGAUUAG GUAACAGUAC UGUAUUAAUG UUAAAUUUCU | |
| | UGAUUUUGUU AAGUGUGCUA UGUAUAAAAC AAUGUUCUUA UUCUUUGGAA | |
| | UACAUUUAGG GGUGAUGGGU UUUUAUCUUC AACUAACUCU CAAAUGAUUA | |
| | AAAAAACCAA UAUGUGUAUA UGUGUAUAAU ACAUAUACAU ACAUACAUAC | |
| | AUACACACAC ACAUACACAC ACACAUACAC AUACACACAC AUAUACAUAC | |
| | ACACACAUAC AUAUAUACAC AUACACAUAC AUGCAUAUAC AUACAUACAC | |
| | AUGCAUACAU ACACACAUAC ACGCGAUACA UACAUACAUA UACAUAUAUA | |
| | CACAUACAUA CACAUACAUA UAUACACAUA CACACACAUU UUUAAAUGCC | |
| | CACCAAAAGU | |
| 64 | AAUUCCAGCG AGAGGCAGAG GGAGCGAGCG GGCGGCCGGC UAGGGUGGAA | cMYC full length(d) IRES |
| | GAGCCGGGCG AGCAGAGCUG CGCUGCGGGC GUCCUGGGAA GGGAGAUCCG | |
| | GAGCGAAUAG GGGGCUUCGC CUCUGGCCCA GCCCUCCCGC UUGAUCCCCC | |
| | AGGCCAGCGG UCCGCAACCC UUGCCGCAUC CACGAAACUU UGCCCAUAGC | |
| | AGCGGGCGGG CACUUUGCAC UGGAACUUAC AACACCCGAG CAAGGACGCG | |
| | ACUCUCCCGA CGCGGGGAGG CUAUUCUGCC CAUUUGGGGA CACUUCCCCG | |
| | CCGCUGCCAG GACCCGCUUC UCUGAAAGGC UCUCCUUGCA GCUGCUUAGA | |
| | CGCUGGAUUU UUUUCGGGUA GUGGAAAACC AGCAGCCUCC CGCGA | |
| 65 | UCGCGGGAGG CUGCUGGUUU UCCACUACCC GAAAAAAAUC CAGCGUCUAA | cMYC full length(i) IRES |
| | GCAGCUGCAA GGAGAGCCUU UCAGAGAAGC GGGUCCUGGC AGCGGCGGGG | |
| | AAGUGUCCCC AAAUGGGCAG AAUAGCCUCC CCGCGUCGGG AGAGUCGCGU | |
| | CCUUGCUCGG GUGUUGUAAG UUCCAGUGCA AAGUGCCCGC CCGCUGCUAU | |
| | GGGCAAAGUU UCGUGGAUGC GGCAAGGGUU GCGGACCGCU GGCCUGGGGG | |
| | AUCAAGCGGG AGGGCUGGGC CAGAGGCGAA GCCCCCUAUU CGCUCCGGAU | |
| | CUCCCUUCCC AGGACGCCCG CAGCGCAGCU CUGCUCGCCC GGCUCUUCCA | |
| | CCCUAGCCGG CCGCCCGCUC GCUCCCUCUG CCUCUCGCUG GAAUU | |
| 66 | GGGCACUUUG CACUGGAACU UACAACACCC GAGCAAGGAC GCGACUCU | cMYC short variant(d) IRES |
| 67 | AGAGUCGCGU CCUUGCUCGG GUGUUGUAAG UUCCAGUGCA AAGUGCCC | cMYC short variant(i) IRES |
| 68 | GUACUGACAU CGUAGAUGGA AAUCAUAAAC UGACUCUUGG UUUGAUUUGG | DMD(d) IRES |
| | AAUAUAAUCC UCCACUGGCA G | |
| 69 | CUGCCAGUGG AGGAUUAUAU UCCAAAUCAA ACCAAGAGUC AGUUUAUGAU | DMD(i) IRES |
| | UUCCAUCUAC GAUGUCAGUA C | |
| 70 | GCATGGCTTCAGCCGAGT | hNURR1 BD1 |
| 71 | GCATGGCTTCAGCCGAGTTACA | hNURR1 BD2 |
| 72 | GGCTTCAGCCGAGTTACA | hNURR1 BD3 |
| 73 | GGCTTCAGCCGAGTTACAGG | hNURR1 BD4 |
| 74 | GCATGGCTTCAGCCGAGTTACAGG | hNURR1 BD5 |
| 75 | CCATGCTAAACTTGACAA | hNURR1 BD6 |
| 76 | GCTAAACTTGACAAACTC | hNURR1 BD7 |
| 77 | CCATGCTAAACTTGACAAACTC | hNURR1 BD8 |
| 78 | CCATAAAGGTACTGAAGC | hNURR1 BD9 |
| 79 | AAAGGTACTGAAGCTGGG | hNURR1 BD10 |
| 80 | CGGCGGCACCTCGGATCG | hGDNF BD1 |
| 81 | TAAAGTCCCGTCCGGCGGCGGCACCTCGGATCGGGTCT | hGDNF BD2 |
| 82 | CTTAAAGTCCCGTGGCGGCACCTCGGATCGGGTCT | hGDNF BD3 |
| 83 | CTTAAAGTCCCGTCGGCGGCAC | hGDNF BD4 |
| 84 | CTTAAAGTCCCGTC | hGDNF BD5 |
| 85 | TCATCTTAAAGTCCCGTC | hGDNF BD6 |
| 86 | TCATCTTAAAGTCCCGTCC | hGDNF BD7 |
| 87 | TCATCTTAAAGTCCCGTCCG | hGDNF BD8 |
| 88 | TCATCTTAAAGTCCCGTCCGGCGGCGGCACC | hGDNF BD9 |
| 89 | TCATCTTAAAGTCCCGTCCGGCGGCGGCACCTCGGATCGGGTCT | hGDNF BD10 |
| 90 | AACTTCATCTTAAAGTGCACCTCGGA | hGDNF BD11 |
| 91 | TCCCATAACTTCATCTTAAAGTCCCGTCCGGCGGCGGCACCTCGGATCGGGTCTCCGCA | hGDNF BD12 |
| | GACCCTAGGTT | |
| 92 | CATCCCATAACTTCATCTTAAAGTCCCGTCC | hGDNF BD13 |
| 93 | TCTTAAAGTCCCGTCCGGCGG | hGDNF BD14 |
| 94 | GCATATTTGAGTCACTGC | hGDNF BD15 |
| 95 | ATTTGAGTCACTGCTCAGCGCGAAG | hGDNF BD16 |
| 96 | CCATGACATCATCGAACTGATCAGGATAATCCTCTGGCATATTTGA | hGDNF BD17 |
| 97 | CCATGACATCATCGAACT | hGDNF BD18 |
| 98 | ACATGCCTGCCCTACTTT | hGDNF BD19 |
| 99 | TCATCTTAAAGTCCCGTCCGGCGGC | hGDNF BD20 |
| 100 | CTTAAAGTCCCGTCCGGCGGC | hGDNF BD21 |
| 101 | ACGACATCCCATAACTTCATC | hGDNF BD22 |
| 102 | ACATCCCATAACTTCATC | hGDNF BD23 |
| 103 | ATTTGAGTCACTGC | hGDNF BD24 |
| 104 | ATTTGAGTCACTGCTCAG | hGDNF BD25 |
| 105 | GCATATTTGAGTCACTGCTCAGCG | hGDNF BD26 |
| 106 | GGGACTGCGGCTAGGGCCGGCGGGTCTGGATGGCGGGTGC | hRET51 BD1 |
| 107 | CCCGTGCGCGCTGGGGCC | hRET51 BD2 |
| 108 | CCGTGCGCGCTGGGGCCACGGCTGGAGGG | hRET51 BD3 |
| 109 | CCCGTGCGCGCTGGGGCCACGGCTGGAGGGACTGCGGC | hRET51 BD4 |
| 110 | CGCCCGTGCGCGCTGGGGCCACGGCTGGAGGGACTGCGGC | hRET51 BD5 |
| 111 | CGCCCGTGCGCGCT | hRET51 BD6 |
| 112 | CCATCGCCCGTGCGCGCT | hRET51 BD7 |
| 113 | CCATCGCCCGTGCGCGCTG | hRET51 BD8 |
| 114 | CCATCGCCCGTGCGCGCTGG | hRET51 BD9 |
| 115 | CCATCGCCCGTGCGCGCTGGGGCCACGGCTGGAGGGACTGCGGC | hRET51 BD10 |
| 116 | TTCGCCATCGCCCGTGCGCGCTGGGGCCACGGCTGGAGGG | hRET51 BD11 |
| 117 | GTCGCCTTCGCCATCGCCCGTGCGCGCTGGGGCCACGGCTGGAGGGACTGCGGCTAGG | hRET51 BD12 |
| | GCCGGCGGGTCT | |
| 118 | CCATCACCACCTCCTCGC | hRET51 BD13 |
| 119 | CACCACCTCCTCGCGCGCGCCGGCGTGCACGGTGCACACG | hRET51 BD14 |
| 120 | GCATGGTGCGGTTCTCCG | hRET51 BD15 |
| 121 | GGTGCGGTTCTCCGAGATGGAGAGGTTCCGGTTGAGAACCAGCCTATAGT | hRET51 BD16 |
| 122 | CCAATGCCACTTTGCCTA | hRET51 BD17 |
| 123 | CGGTTGCGGACACTGAGC | hRET51 BD18 |
| 124 | CACGGTGTCCTCCTTCCG | hRET51 BD19 |
| 125 | TGTCCTCCTTCCGCTTGAACTCCACCACGGCGCTGGCGGTGTCG | hRET51 BD20 |
| 126 | GCCATCGCCCGTGCGCGC | hRET51 BD21 |
| 127 | CCTCCTTCCGCTTGAACTCCACCA | hRET51 BD22 |
| 128 | CATCGCCCGTGCGCGCT | hRET51 BD23 |
| 129 | CCATCGCCCGTGCGC | hRET51 BD24 |
| 130 | TCGCCATCGCCCGTGCGC | hRET51 BD25 |
| 131 | CTTCGCCATCGCCCGTGCGC | hRET51 BD26 |
| 132 | GACGTCGCCTTCGCCATC | hRET51 BD27 |
| 133 | CGGACGTCGCCTTCGCCATC | hRET51 BD28 |
| 134 | GCACCGGACGTCGCCTTCGCCATC | hRET51 BD29 |
| 135 | CACCATCACCACCTCCTCGC | hRET51 BD30 |
| 136 | GGAAGGGCACCATCACCA | hRET51 BD31 |
| 137 | AAGGGCACCATCACC | hRET51 BD32 |
| 138 | GAGGATCCACGTCGGCGA | hGBA BD1 |
| 139 | CCTCAGGGTCATTAGATG | hGBA BD2 |
| 140 | CCTCAGGGTCATTAGATGAAGAGAAGACCACAGGGGTT | hGBA BD3 |
| 141 | CCCCTCAGGGTCATTAGATGAAGAGAAGACCACAGGGGTT | hGBA BD4 |
| 142 | CCATCCCCTCAGGGTCATTAGATGAAGAGAAGACCACAGGGGTT | hGBA BD5 |
| 143 | CCCCTCAGGGTCAT | hGBA BD6 |
| 144 | CCATCCCCTCAGGGTCAT | hGBA BD7 |
| 145 | CCATCCCCTCAGGGTCATT | hGBA BD8 |
| 146 | CCATCCCCTCAGGGTCATTA | hGBA BD9 |
| 147 | AACTCCATCCCCTCAGGGTCATTAGATGAAGAGAAGACCA | hGBA BD10 |
| 148 | CTTGAAAACTCCATCCCCTCAGGGTCATTAGATGAAGAGAAGACCACAGGGGTTCCAGAG | hGBA BD11 |
| | TCTCTGAAGG | |
| 149 | GATGCTTACCCTACTCAAAGGCTTGGGACATTCCTCTCTG | hGBA BD12 |
| 150 | CCATCCGTCGCCCACTGC | hGBA BD13 |
| 151 | CCGTCGCCCACTGCGTGTACTCTCATAGCGGCTGAAGGTA | hGBA BD14 |
| 152 | ATACTCAGCTCCATCCGTCGCCCACTGCGTGTACTCTCATAGCGGCTGAAGGTA | hGBA BD15 |
| 153 | GCATCCCTCGATCCCAGG | hGBA BD16 |
| 154 | CAGGGGTATCTTGAGCTT | hGBA BD17 |
| 155 | GTATCTTGAGCTTGGTAT | hGBA BD18 |
| 156 | ACTCAGCTCCATCCGTCG | hGBA BD19 |
| 157 | CTGGCCATGGGTACCCGGATGATGTTATAT | hGBA BD20 |
| 158 | TGCTCCACACGGCTCCCT | mSNCA BD1 |
| 159 | CTAAAGATGTATTTTTGCTCCACACGGCTCCCTAGGCT | mSNCA BD2 |
| 160 | TATTTTTGCTCCACACGGCTCCCTAGGCTT | mSNCA BD3 |
| 161 | CTAAAGATGTATTTTTGC | mSNCA BD4 |
| 162 | GGCTAAAGATGTATTTTTGCTCCACACGGCTCCCTAGGCT | mSNCA BD5 |
| 163 | GGCTAAAGATGTATTTTTGCTCCACAC | mSNCA BD6 |
| 164 | GGCTAAAGATGTAT | mSNCA BD7 |
| 165 | CCATGGCTAAAGATGTAT | mSNCA BD8 |
| 166 | CCATGGCTAAAGATGTATT | mSNCA BD9 |
| 167 | CCATGGCTAAAGATGTATTT | mSNCA BD10 |
| 168 | CCATGGCTAAAGATGTATTTTTGCTCCACACGGCTCCCTAGGCT | mSNCA BD11 |
| 169 | ACATCCATGGCTAAAGATGTATTTTTGCTCCACACGGCTC | mSNCA BD12 |
| 170 | TCATGAACACATCCATGGCTAAAGATGTATTTTTGCTCCACACGGCTCCCTAGGCTTCTGA | mSNCA BD13 |
| | AGAACTCCG | |
| 171 | TCATGAACACATCCATGGCTAAAGATGTATTTTTGCTCCACA | mSNCA BD14 |
| 172 | TGGCTAAAGATGTATTTTTGC | mSNCA BD15 |
| 173 | TCATGAACACATCCATGG | mSNCA BD16 |
| 174 | GAACACATCCATGGCTAAAGATGTATTTTTGCTCCACACG | mSNCA BD17 |
| 175 | CTCCTCACCCTTGCCCAT | mSNCA BD18 |
| 176 | TACCCCTCCTCACCCTTGCCCATCTGGTCCTTCTTGA | mSNCA BD19 |
| 177 | GCATTTCATAAGCCTCACTGCCAGGATCCACAGGCATGTCTTCCAG | mSNCA BD20 |
| 178 | TTTTGGAACCTACATAGA | mSNCA BD21 |
| 179 | CACTGTTGTCACTCCATGAACCACTCCTTCCTTAGTTTTGGAAC | mSNCA BD22 |
| 180 | TACACCACACTGTCGTCG | hSNCA BD1 |
| 181 | CTAATGAATTCCTTTACACCACACTGTCGTCGAATGGC | hSNCA BD2 |
| 182 | TTCCTTTACACCACACTGTCGTCGAATGGC | hSNCA BD3 |
| 183 | CTAATGAATTCCTTTACA | hSNCA BD4 |
| 184 | GGCTAATGAATTCCTTTACACCACACTGTCGTCGAATGGC | hSNCA BD5 |
| 185 | GGCTAATGAATTCCTTTACACCACAC | hSNCA BD6 |
| 186 | GGCTAATGAATTCC | hSNCA BD7 |
| 187 | CCATGGCTAATGAATTCC | hSNCA BD8 |
| 188 | CCATGGCTAATGAATTCCT | hSNCA BD9 |
| 189 | CCATGGCTAATGAATTCCTT | hSNCA BD10 |
| 190 | CCATGGCTAATGAATTCCTTTACACCACACTGTCGTCGAATGGC | hSNCA BD11 |
| 191 | ACATCCATGGCTAATGAATTCCTTTACACCACACTGTCGT | hSNCA BD12 |
| 192 | TCATGAATACATCCATGGCTAATGAATTCCTTTACACCACACTGTCGTCGAATGGCCACTC | hSNCA BD13 |
| | CCAGTTCTC | |
| 193 | TCATGAATACATCCATGGCTAATGAATTCCTTTACACCACA | hSNCA BD14 |
| 194 | TGGCTAATGAATTCCTTTACA | hSNCA BD15 |
| 195 | TCATGAATACATCCATGG | hSNCA BD16 |
| 196 | GAATACATCCATGGCTAATGAATTCCTTTACACCACACTG | hSNCA BD17 |
| 197 | GCATTTCATAAGCCTCATTGTCAGGATCCACAGGCATATCTTCCAG | hSNCA BD18 |
| 198 | GCATATCTTCCAGAATTC | hSNCA BD19 |
| 199 | TTTTGGAGCCTACATAGA | hSNCA BD20 |
| 200 | GAGCCTACATAGAGAACACCCTCTTTTGTCTTTCCTGCTGCTTC | hSNCA BD21 |
| 201 | TTCTTCATTCTTGCCCAA | hSNCA BD22 |
| 202 | GAATACATCCATGGCTAA | hSNCA BD23 |
| 203 | AUCUGCAGAAUUC | Linker |
| 204 | GAAUUC | Short Linker |
| 205 | CTTAAAGTCCCGTC | GDNF BDI (−14/−1 M1) |
| 206 | TCATCTTAAAGTCCCGTC | GDNF BDII (−14/+4 M1) |
| 207 | TCATCTTAAAGTCCCGTCC | GDNF BDIII (−15/+4 M1) |
| 208 | GCATATTTGAGTCACTGC | GDNF BDIV (−14/+4 M2) |
| 209 | ATTTGAGTCACTGCTCAGCGCGAAG | GDNF BDV (−25/−1 M2) |
| 210 | CCATGACATCATCGAACTGATCAGGATAATCCTCTGGCATATTTGA | GDNF BDVI (−42/+4 M3) |

1.-23. (canceled)

24. A functional nucleic acid molecule comprising:
at least one target binding sequence comprising a sequence reverse complementary to a target mRNA sequence selected from the group consisting of: a Nurr1, GDNF, cRET or GBA mRNA sequence; and
at least one regulatory sequence comprising a SINE B2 element, a functionally active fragment of a SINE B2 element, an internal ribosome entry site (IRES) sequence or a functionally active fragment of an internal ribosome entry site (IRES) sequence.

25. The functional nucleic acid molecule according claim 24, wherein the at least one target binding sequence is at least 18 nucleotides long and comprises, from 3′ to 5′:
a sequence reverse complementary to 0 to 20 nucleotides of the 5′ UTR and 0 to 4 nucleotides of the CDS of the Nurr1 mRNA sequence; or
a sequence reverse complementary to 0 to 18 nucleotides of the region upstream of an AUG site (start codon) of the Nurr1 mRNA and 0 to 4 nucleotides of the CDS of the Nurr1 mRNA sequence downstream of said AUG site.

26. The functional nucleic acid molecule according to claim 24, wherein the at least one target binding sequence is at least 14 nucleotides long and comprises, from 3′ to 5′:
a sequence reverse complementary to 0 to 21 nucleotides of the 5′ UTR and 0 to 20 nucleotides of the CDS of the GDNF mRNA sequence; or
a sequence reverse complementary to 0 to 20 nucleotides of the region upstream of an AUG site (start codon) of the GDNF mRNA and 0 to 4 nucleotides of the CDS of the GDNF mRNA sequence downstream of said AUG site.

27. The functional nucleic acid molecule according to claim 24, wherein the at least one target binding sequence is at least 15 nucleotides long and comprises, from 3′ to 5′:
a sequence reverse complementary to 0 to 14 nucleotides of the 5′ UTR and 0 to 23 nucleotides of the CDS of the cRET mRNA sequence; or
a sequence reverse complementary to 0 to 14 nucleotides of the region upstream of an AUG site (start codon) of the cRET mRNA and 0 to 13 nucleotides of the CDS of the cRET mRNA sequence downstream of said AUG site.

28. The functional nucleic acid molecule according to claim 24, wherein the at least one target binding sequence is at least 15 nucleotides long and comprises, from 3′ to 5′:
a sequence reverse complementary to 0 to 56 nucleotides of the 5′ UTR and 0 to 14 nucleotides of the CDS of the GBA mRNA sequence; or
a sequence reverse complementary to 0 to 40 nucleotides of the region upstream of an AUG site (start codon) of the GBA mRNA and 0 to 12 nucleotides of the CDS of the GBA mRNA sequence downstream of said AUG site.

29. The functional nucleic acid molecule according to claim 24, wherein the at least one regulatory sequence comprises a sequence with at least 75% sequence identity with a sequence selected from the group consisting of SEQ ID NOs: 1-69.

30. The functional nucleic acid molecule according to claim 24, wherein the functional nucleic acid molecule increases translation of an endogenous mRNA sequence selected from the group consisting of: an endogenous Nurr1, human GDNF, cRET or GBA mRNA sequence.

31. A DNA molecule encoding the functional nucleic acid molecule according to claim 24.

32. An expression vector comprising the functional nucleic acid molecule according to claim 24 or a DNA molecule encoding the functional nucleic acid molecule according to claim 24.

33. A composition comprising (a) the functional nucleic acid molecule according to claim 24, (b) a DNA molecule encoding the functional nucleic acid molecule according to claim 24 or (c) an expression vector comprising (a) or (b).

34. A pharmaceutical composition comprising the composition of claim 32, in admixture with a suitable pharmaceutical excipient, diluent or carrier.

35. A method of treating a disease or disorder of the nervous system comprising administering to a subject with a disease or disorder of the nervous system, a therapeutically effective amount (a) the functional nucleic acid molecule according to claim 24, (b) a DNA molecule encoding the functional nucleic acid molecule according to claim 24, (c) an expression vector comprising (a) or (b), (d) a composition comprising (a), (b) or (c), or (e) a pharmaceutical composition comprising (d) in admixture with a suitable pharmaceutical excipient, diluent or carrier.

36. The method of treating a disease or disorder of the nervous system according to claim 35, wherein the disease or disorder of the nervous system is a disease or disorder of the central nervous system.

37. The method of treating a disease or disorder of the nervous system according to claim 35, wherein the disease or disorder of the nervous system is a neurodegenerative disease.

38. The method of treating a disease or disorder of the nervous system according to claim 35, wherein the disease or disorder of the nervous system is Parkinson's disease.

39. A method of treating a disease or disorder of the nervous system comprising administering a therapeutic agent that increases translation of an endogenous mRNA sequence selected from the group consisting of: an endogenous Nurr1, GDNF, cRET or GBA mRNA sequence, to a subject with a disease or disorder of the nervous system, to a subject with a disease or disorder of the nervous system.

40. A method for increasing the protein synthesis efficiency of Nurr1, GDNF, cRET or GBA in a cell comprising administering to the cell, (a) the functional nucleic acid molecule according to claim 24, (b) a DNA molecule encoding the functional nucleic acid molecule according to claim 24, (c) an expression vector comprising (a) or (b), (d) a composition comprising (a), (b) or (c), or (e) a pharmaceutical composition comprising (d) in admixture with a suitable pharmaceutical excipient, diluent or carrier.

41. A method of producing a non-human animal model of Parkinson's disease, comprising: administering a functional nucleic acid molecule comprising: at least one target binding sequence comprising a sequence reverse complementary to a SNCA mRNA sequence; and at least one regulatory sequence comprising a SINE B2 element, a functionally active fragment of a SINE B2 element, an IRES sequence or a functionally active fragment of an IRES sequence, to a non-human animal.

42. The method according to claim 41, wherein the non-human animal is a non-human primate.

43. A method of producing a non-human animal model of Parkinson's disease, comprising: administering to a non-human animal, (a) the functional nucleic acid molecule according to claim 24, (b) a DNA molecule encoding the functional nucleic acid molecule according to claim 24, (c) an expression vector comprising (a) or (b), (d) a composition comprising (a), (b) or (c), or (e) a pharmaceutical composition comprising (d) in admixture with a suitable pharmaceutical excipient, diluent or carrier;
wherein the functional nucleic acid molecule comprises:
at least one target binding sequence comprising a sequence reverse complementary to a SNCA mRNA sequence; and
at least one regulatory sequence comprising a SINE B2 element, a functionally active fragment of a SINE B2 element, an IRES sequence or a functionally active fragment of an IRES sequence.

44. The method according to claim 43, wherein the non-human animal is a non-human primate.
